SEQUENCE LISTING (164 sequences)

SEQ ID NO: 1 - DNA, 32 bp, synthetic construct
  source 1..32: mol_type = other DNA
  misc_feature 20: note = DNA
  misc_feature order(1..19,21..32): note = RNA
  modified_base order(1..5,18,23): mod_base = OTHER; note = 2&apos;-O-methoxyethyl
  modified_base order(6..11,21,30..32): mod_base = OTHER; note = 2&apos;-O-methyl
  modified_base order(12..17,22,24..29): mod_base = OTHER; note = 2&apos;-fluoro
  modified_base order(1^2,31^32): mod_base = OTHER; note = (1,3-dimethylimidazolidin-2-ylidene) phosphoramidate linkage
  modified_base order(6..18,19^20,22..31): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase
cctgggtgctccacctggnacgtatatctctg

SEQ ID NO: 2 - DNA, 32 bp, synthetic construct; identical to SEQ ID NO: 1
  source 1..32: mol_type = other DNA
  misc_feature 20: note = DNA
  misc_feature order(1..19,21..32): note = RNA
  modified_base order(1..5,18,23): mod_base = OTHER; note = 2&apos;-O-methoxyethyl
  modified_base order(6..12,14,16,21,25,27,29..32): mod_base = OTHER; note = 2&apos;-O-methyl
  modified_base order(13,15,17,22,24,26,28): mod_base = OTHER; note = 2&apos;-fluoro
  modified_base order(1^2,31^32): mod_base = OTHER; note = (1,3-dimethylimidazolidin-2-ylidene) phosphoramidate linkage
  modified_base order(6..18,19^20,22..31): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase

SEQ ID NO: 3 - DNA, 32 bp, synthetic construct
  source 1..32: mol_type = other DNA
  misc_feature 20: note = DNA
  misc_feature order(1..19,21..32): note = RNA
  modified_base order(1..5,18,23): mod_base = OTHER; note = 2&apos;-O-methoxyethyl
  modified_base order(6..11,21,30..32): mod_base = OTHER; note = 2&apos;-O-methyl
  modified_base order(12..17,22,24..29): mod_base = OTHER; note = 2&apos;-fluoro
  modified_base 19: mod_base = OTHER; note = 2&apos;,2&apos;-difluoro
  modified_base order(1^2,31^32): mod_base = OTHER; note = (1,3-dimethylimidazolidin-2-ylidene) phosphoramidate linkage
  modified_base order(6..18,19^20,22..31): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
cctgggtgctccacctggcacgtatatctctg

SEQ ID NO: 4 - DNA, 32 bp, synthetic construct; identical to SEQ ID NO: 3
  source 1..32: mol_type = other DNA
  misc_feature 20: note = DNA
  misc_feature order(1..19,21..32): note = RNA
  modified_base order(1..5,18,23): mod_base = OTHER; note = 2&apos;-O-methoxyethyl
  modified_base order(6..12,14,16,21,25,27,29..32): mod_base = OTHER; note = 2&apos;-O-methyl
  modified_base order(13,15,17,22,24,26,28): mod_base = OTHER; note = 2&apos;-fluoro
  modified_base 19: mod_base = OTHER; note = 2&apos;,2&apos;-difluoro
  modified_base order(1^2,31^32): mod_base = OTHER; note = (1,3-dimethylimidazolidin-2-ylidene) phosphoramidate linkage
  modified_base order(6..18,19^20,22..31): mod_base = OTHER; note = phosphorothioate
  modified_base 20^21: mod_base = OTHER; note = methylphosphonate
  modified_base 1..2: mod_base = m5c
  modified_base order(3,23): mod_base = m5u

SEQ ID NO: 5 - DNA, 32 bp, synthetic construct; identical to SEQ ID NO: 1
  source 1..32: mol_type = other DNA
  misc_feature 20: note = DNA
  misc_feature order(1..19,21..32): note = RNA
  modified_base order(1..5,18,23): mod_base = OTHER; note = 2&apos;-O-methoxyethyl
  modified_base order(6..11,21,30..32): mod_base = OTHER; note = 2&apos;-O-methyl
  modified_base order(12..17,22,24..29): mod_base = OTHER; note = 2&apos;-fluoro
  modified_base order(1^2,31^32): mod_base = OTHER; note = (1,3-dimethylimidazolidin-2-ylidene) phosphoramidate linkage
  modified_base order(6..20,21..31): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase

SEQ ID NO: 6 - DNA, 32 bp, synthetic construct; identical to SEQ ID NO: 1
  source 1..32: mol_type = other DNA
  misc_feature 20: note = DNA
  misc_feature order(1..19,21..32): note = RNA
  modified_base order(1..5,18,23): mod_base = OTHER; note = 2&apos;-O-methoxyethyl
  modified_base order(6..12,14,16,21,25,27,29..32): mod_base = OTHER; note = 2&apos;-O-methyl
  modified_base order(13,15,17,22,24,26,28): mod_base = OTHER; note = 2&apos;-fluoro
  modified_base order(1^2,31^32): mod_base = OTHER; note = (1,3-dimethylimidazolidin-2-ylidene) phosphoramidate linkage
  modified_base order(6..20,21..31): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase

SEQ ID NO: 7 - DNA, 32 bp, synthetic construct; identical to SEQ ID NO: 3
  source 1..32: mol_type = other DNA
  misc_feature 20: note = DNA
  misc_feature order(1..19,21..32): note = RNA
  modified_base order(1..5,18,23): mod_base = OTHER; note = 2&apos;-O-methoxyethyl
  modified_base order(6..11,21,30..32): mod_base = OTHER; note = 2&apos;-O-methyl
  modified_base order(12..17,22,24..29): mod_base = OTHER; note = 2&apos;-fluoro
  modified_base 19: mod_base = OTHER; note = 2&apos;,2&apos;-difluoro
  modified_base order(1^2,31^32): mod_base = OTHER; note = (1,3-dimethylimidazolidin-2-ylidene) phosphoramidate linkage
  modified_base order(6..20,21..31): mod_base = OTHER; note = phosphorothioate
  modified_base 20^21: mod_base = OTHER; note = methylphosphonate
  modified_base 1..2: mod_base = m5c
  modified_base order(3,23): mod_base = m5u

SEQ ID NO: 8 - DNA, 32 bp, synthetic construct; identical to SEQ ID NO: 3
  source 1..32: mol_type = other DNA
  misc_feature 20: note = DNA
  misc_feature order(1..19,21..32): note = RNA
  modified_base order(1..5,18,23): mod_base = OTHER; note = 2&apos;-O-methoxyethyl
  modified_base order(6..12,14,16,21,25,27,29..32): mod_base = OTHER; note = 2&apos;-O-methyl
  modified_base order(13,15,17,22,24,26,28): mod_base = OTHER; note = 2&apos;-fluoro
  modified_base 19: mod_base = OTHER; note = 2&apos;,2&apos;-difluoro
  modified_base order(1^2,31^32): mod_base = OTHER; note = (1,3-dimethylimidazolidin-2-ylidene) phosphoramidate linkage
  modified_base order(6..20,21..31): mod_base = OTHER; note = phosphorothioate
  modified_base 20^21: mod_base = OTHER; note = methylphosphonate
  modified_base 1..2: mod_base = m5c
  modified_base order(3,23): mod_base = m5u

SEQ ID NO: 9 - DNA, 30 bp, synthetic construct
  source 1..30: mol_type = other DNA
  misc_feature 18: note = DNA
  misc_feature order(1..17,19..30): note = RNA
  modified_base order(1..5,16,21): mod_base = OTHER; note = 2&apos;-O-methoxyethyl
  modified_base order(6..9,19,28..30): mod_base = OTHER; note = 2&apos;-O-methyl
  modified_base order(10..15,20,22..27): mod_base = OTHER; note = 2&apos;-fluoro
  modified_base order(1^2,29^30): mod_base = OTHER; note = (1,3-dimethylimidazolidin-2-ylidene) phosphoramidate linkage
  modified_base order(6..16,17^18,20..29): mod_base = OTHER; note = phosphorothioate
  modified_base 18^19: mod_base = OTHER; note = methylphosphonate
  modified_base order(1,5,21): mod_base = m5u
  modified_base 17: mod_base = OTHER; note = 6-amino-5-nitro-3-yl-2(1H)-pyridone nucleobase
tgggtgctccacctggnacgtatatctctg

SEQ ID NO: 10 - DNA, 30 bp, synthetic construct; identical to SEQ ID NO: 9
  source 1..30: mol_type = other DNA
  misc_feature 18: note = DNA
  misc_feature order(1..17,19..30): note = RNA
  modified_base order(1..5,16,21): mod_base = OTHER; note = 2&apos;-O-methoxyethyl
  modified_base order(6..10,12,14,19,23,25,27..30): mod_base = OTHER; note = 2&apos;-O-methyl
  modified_base order(11,13,15,20,22,24,26): mod_base = OTHER; note = 2&apos;-fluoro
  modified_base order(1^2,29^30): mod_base = OTHER; note = (1,3-dimethylimidazolidin-2-ylidene) phosphoramidate linkage
  modified_base order(6..16,17^18,20..29): mod_base = OTHER; note = phosphorothioate
  modified_base 18^19: mod_base = OTHER; note = methylphosphonate
  modified_base order(1,5,21): mod_base = m5u
  modified_base 17: mod_base = OTHER; note = 6-amino-5-nitro-3-yl-2(1H)-pyridone nucleobase

SEQ ID NO: 11 - DNA, 30 bp, synthetic construct
  source 1..30: mol_type = other DNA
  misc_feature 18: note = DNA
  misc_feature order(1..17,19..30): note = RNA
  modified_base order(1..5,16,21): mod_base = OTHER; note = 2&apos;-O-methoxyethyl
  modified_base order(6..9,19,28..30): mod_base = OTHER; note = 2&apos;-O-methyl
  modified_base order(10..15,20,22..27): mod_base = OTHER; note = 2&apos;-fluoro
  modified_base 17: mod_base = OTHER; note = 2&apos;,2&apos;-difluoro
  modified_base order(1^2,29^30): mod_base = OTHER; note = (1,3-dimethylimidazolidin-2-ylidene) phosphoramidate linkage
  modified_base order(6..16,17^18,20..29): mod_base = OTHER; note = phosphorothioate
  modified_base 18^19: mod_base = OTHER; note = methylphosphonate
  modified_base order(1,5,21): mod_base = m5u
tgggtgctccacctggcacgtatatctctg

SEQ ID NO: 12 - DNA, 30 bp, synthetic construct; identical to SEQ ID NO: 11
  source 1..30: mol_type = other DNA
  misc_feature 18: note = DNA
  misc_feature order(1..17,19..30): note = RNA
  modified_base order(1..5,16,21): mod_base = OTHER; note = 2&apos;-O-methoxyethyl
  modified_base order(6..10,12,14,19,23,25,27..30): mod_base = OTHER; note = 2&apos;-O-methyl
  modified_base order(11,13,15,20,22,24,26): mod_base = OTHER; note = 2&apos;-fluoro
  modified_base 17: mod_base = OTHER; note = 2&apos;,2&apos;-difluoro
  modified_base order(1^2,29^30): mod_base = OTHER; note = (1,3-dimethylimidazolidin-2-ylidene) phosphoramidate linkage
  modified_base order(6..16,17^18,20..29): mod_base = OTHER; note = phosphorothioate
  modified_base 18^19: mod_base = OTHER; note = methylphosphonate
  modified_base order(1,5,21): mod_base = m5u

SEQ ID NO: 13 - DNA, 30 bp, synthetic construct; identical to SEQ ID NO: 9
  source 1..30: mol_type = other DNA
  misc_feature 18: note = DNA
  misc_feature order(1..17,19..30): note = RNA
  modified_base order(1..5,16,21): mod_base = OTHER; note = 2&apos;-O-methoxyethyl
  modified_base order(6..9,19,28..30): mod_base = OTHER; note = 2&apos;-O-methyl
  modified_base order(10..15,20,22..27): mod_base = OTHER; note = 2&apos;-fluoro
  modified_base order(1^2,29^30): mod_base = OTHER; note = (1,3-dimethylimidazolidin-2-ylidene) phosphoramidate linkage
  modified_base order(6..18,19..29): mod_base = OTHER; note = phosphorothioate
  modified_base 18^19: mod_base = OTHER; note = methylphosphonate
  modified_base order(1,5,21): mod_base = m5u
  modified_base 17: mod_base = OTHER; note = 6-amino-5-nitro-3-yl-2(1H)-pyridone nucleobase

SEQ ID NO: 14 - DNA, 30 bp, synthetic construct; identical to SEQ ID NO: 9
  source 1..30: mol_type = other DNA
  misc_feature 18: note = DNA
  misc_feature order(1..17,19..30): note = RNA
  modified_base order(1..5,16,21): mod_base = OTHER; note = 2&apos;-O-methoxyethyl
  modified_base order(6..10,12,14,19,23,25,27..30): mod_base = OTHER; note = 2&apos;-O-methyl
  modified_base order(11,13,15,20,22,24,26): mod_base = OTHER; note = 2&apos;-fluoro
  modified_base order(1^2,29^30): mod_base = OTHER; note = (1,3-dimethylimidazolidin-2-ylidene) phosphoramidate linkage
  modified_base order(6..18,19..29): mod_base = OTHER; note = phosphorothioate
  modified_base 18^19: mod_base = OTHER; note = methylphosphonate
  modified_base order(1,5,21): mod_base = m5u
  modified_base 17: mod_base = OTHER; note = 6-amino-5-nitro-3-yl-2(1H)-pyridone nucleobase

SEQ ID NO: 15 - DNA, 30 bp, synthetic construct; identical to SEQ ID NO: 11
  source 1..30: mol_type = other DNA
  misc_feature 18: note = DNA
  misc_feature order(1..17,19..30): note = RNA
  modified_base order(1..5,16,21): mod_base = OTHER; note = 2&apos;-O-methoxyethyl
  modified_base order(6..9,19,28..30): mod_base = OTHER; note = 2&apos;-O-methyl
  modified_base order(10..15,20,22..27): mod_base = OTHER; note = 2&apos;-fluoro
  modified_base 17: mod_base = OTHER; note = 2&apos;,2&apos;-difluoro
  modified_base order(1^2,29^30): mod_base = OTHER; note = (1,3-dimethylimidazolidin-2-ylidene) phosphoramidate linkage
  modified_base order(6..18,19..29): mod_base = OTHER; note = phosphorothioate
  modified_base 18^19: mod_base = OTHER; note = methylphosphonate
  modified_base order(1,5,21): mod_base = m5u

SEQ ID NO: 16 - DNA, 30 bp, synthetic construct; identical to SEQ ID NO: 11
  source 1..30: mol_type = other DNA
  misc_feature 18: note = DNA
  misc_feature order(1..17,19..30): note = RNA
  modified_base order(1..5,16,21): mod_base = OTHER; note = 2&apos;-O-methoxyethyl
  modified_base order(6..10,12,14,19,23,25,27..30): mod_base = OTHER; note = 2&apos;-O-methyl
  modified_base order(11,13,15,20,22,24,26): mod_base = OTHER; note = 2&apos;-fluoro
  modified_base 17: mod_base = OTHER; note = 2&apos;,2&apos;-difluoro
  modified_base order(1^2,29^30): mod_base = OTHER; note = (1,3-dimethylimidazolidin-2-ylidene) phosphoramidate linkage
  modified_base order(6..18,19..29): mod_base = OTHER; note = phosphorothioate
  modified_base 18^19: mod_base = OTHER; note = methylphosphonate
  modified_base order(1,5,21): mod_base = m5u

SEQ ID NO: 17 - DNA, 28 bp, synthetic construct
  source 1..28: mol_type = other DNA
  misc_feature 16: note = DNA
  misc_feature order(1..15,17..28): note = RNA
  modified_base order(1..5,14,19): mod_base = OTHER; note = 2&apos;-O-methoxyethyl
  modified_base order(6..7,17,26..28): mod_base = OTHER; note = 2&apos;-O-methyl
  modified_base order(8..13,18,20..25): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(6..14,15^16,18..27): mod_base = OTHER; note = phosphorothioate
  modified_base 16^17: mod_base = OTHER; note = methylphosphonate
  modified_base order(3,19): mod_base = m5u
  modified_base 5: mod_base = m5c
  modified_base 15: mod_base = OTHER; note = 6-amino-5-nitro-3-yl-2(1H)-pyridone nucleobase
ggtgctccacctggnacgtatatctctg

SEQ ID NO: 18 - DNA, 28 bp, synthetic construct
  source 1..28: mol_type = other DNA
  misc_feature 16: note = DNA
  misc_feature order(1..15,17..28): note = RNA
  modified_base order(1..5,14,19): mod_base = OTHER; note = 2&apos;-O-methoxyethyl
  modified_base order(6..8,10,12,17,21,23,25..28): mod_base = OTHER; note = 2&apos;-O-methyl
  modified_base order(9,11,13,18,20,22,24): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(6..14,15^16,18..27): mod_base = OTHER; note = phosphorothioate
  modified_base 16^17: mod_base = OTHER; note = methylphosphonate
  modified_base order(3,19): mod_base = m5u
  modified_base 5: mod_base = m5c
  modified_base 15: mod_base = OTHER; note = 6-amino-5-nitro-3-yl-2(1H)-pyridone nucleobase
ggtgctccacctggnacgtatatctctg

SEQ ID NO: 19 - DNA, 28 bp, synthetic construct
  source 1..28: mol_type = other DNA
  misc_feature 16: note = DNA
  misc_feature order(1..15,17..28): note = RNA
  modified_base order(1..5,14,19): mod_base = OTHER; note = 2&apos;-O-methoxyethyl
  modified_base order(6..7,17,26..28): mod_base = OTHER; note = 2&apos;-O-methyl
  modified_base order(8..13,18,20..25): mod_base = OTHER; note = 2&apos;-fluoro
  modified_base 15: mod_base = OTHER; note = 2&apos;,2&apos;-difluoro
  modified_base order(1^2,27^28): mod_base = OTHER; note = (1,3-dimethylimidazolidin-2-ylidene) phosphoramidate linkage
  modified_base order(6..14,15^16,18..27): mod_base = OTHER; note = phosphorothioate
  modified_base 16^17: mod_base = OTHER; note = methylphosphonate
  modified_base order(3,19): mod_base = m5u
  modified_base 5: mod_base = m5c
ggtgctccacctggcacgtatatctctg

SEQ ID NO: 20 - DNA, 28 bp, synthetic construct
  source 1..28: mol_type = other DNA
  misc_feature 16: note = DNA
  misc_feature order(1..15,17..28): note = RNA
  modified_base order(1..5,14,19): mod_base = OTHER; note = 2&apos;-O-methoxyethyl
  modified_base order(6..8,10,12,17,21,23,25..28): mod_base = OTHER; note = 2&apos;-O-methyl
  modified_base order(9,11,13,18,20,22,24): mod_base = OTHER; note = 2&apos;-fluoro
  modified_base 15: mod_base = OTHER; note = 2&apos;,2&apos;-difluoro
  modified_base order(1^2,27^28): mod_base = OTHER; note = (1,3-dimethylimidazolidin-2-ylidene) phosphoramidate linkage
  modified_base order(6..14,15^16,18..27): mod_base = OTHER; note = phosphorothioate
  modified_base 16^17: mod_base = OTHER; note = methylphosphonate
  modified_base order(3,19): mod_base = m5u
  modified_base 5: mod_base = m5c
ggtgctccacctggcacgtatatctctg

SEQ ID NO: 21 - DNA, 28 bp, synthetic construct
  source 1..28: mol_type = other DNA
  misc_feature 16: note = DNA
  misc_feature order(1..15,17..28): note = RNA
  modified_base order(1..5,14,19): mod_base = OTHER; note = 2&apos;-O-methoxyethyl
  modified_base order(6..7,17,26..28): mod_base = OTHER; note = 2&apos;-O-methyl
  modified_base order(8..13,18,20..25): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(6..16,17..27): mod_base = OTHER; note = phosphorothioate
  modified_base 16^17: mod_base = OTHER; note = methylphosphonate
  modified_base order(3,19): mod_base = m5u
  modified_base 5: mod_base = m5c
  modified_base 15: mod_base = OTHER; note = 6-amino-5-nitro-3-yl-2(1H)-pyridone nucleobase
ggtgctccacctggnacgtatatctctg

SEQ ID NO: 22 - DNA, 28 bp, synthetic construct
  source 1..28: mol_type = other DNA
  misc_feature 16: note = DNA
  misc_feature order(1..15,17..28): note = RNA
  modified_base order(1..5,14,19): mod_base = OTHER; note = 2&apos;-O-methoxyethyl
  modified_base order(6..8,10,12,17,21,23,25..28): mod_base = OTHER; note = 2&apos;-O-methyl
  modified_base order(9,11,13,18,20,22,24): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(6..16,17..27): mod_base = OTHER; note = phosphorothioate
  modified_base 16^17: mod_base = OTHER; note = methylphosphonate
  modified_base order(3,19): mod_base = m5u
  modified_base 5: mod_base = m5c
  modified_base 15: mod_base = OTHER; note = 6-amino-5-nitro-3-yl-2(1H)-pyridone nucleobase
ggtgctccacctggnacgtatatctctg

SEQ ID NO: 23 - DNA, 28 bp, synthetic construct
  source 1..28: mol_type = other DNA
  misc_feature 16: note = DNA
  misc_feature order(1..15,17..28): note = RNA
  modified_base order(1..5,14,19): mod_base = OTHER; note = 2&apos;-O-methoxyethyl
  modified_base order(6..7,17,26..28): mod_base = OTHER; note = 2&apos;-O-methyl
  modified_base order(8..13,18,20..25): mod_base = OTHER; note = 2&apos;-fluoro
  modified_base 15: mod_base = OTHER; note = 2&apos;,2&apos;-difluoro
  modified_base order(1^2,27^28): mod_base = OTHER; note = (1,3-dimethylimidazolidin-2-ylidene) phosphoramidate linkage
  modified_base order(6..16,17..27): mod_base = OTHER; note = phosphorothioate
  modified_base 16^17: mod_base = OTHER; note = methylphosphonate
  modified_base order(3,19): mod_base = m5u
  modified_base 5: mod_base = m5c
ggtgctccacctggcacgtatatctctg

SEQ ID NO: 24 - DNA, 28 bp, synthetic construct
  source 1..28: mol_type = other DNA
  misc_feature 16: note = DNA
  misc_feature order(1..15,17..28): note = RNA
  modified_base order(1..5,14,19): mod_base = OTHER; note = 2&apos;-O-methoxyethyl
  modified_base order(6..8,10,12,17,21,23,25..28): mod_base = OTHER; note = 2&apos;-O-methyl
  modified_base order(9,11,13,18,20,22,24): mod_base = OTHER; note = 2&apos;-fluoro
  modified_base 15: mod_base = OTHER; note = 2&apos;,2&apos;-difluoro
  modified_base order(1^2,27^28): mod_base = OTHER; note = (1,3-dimethylimidazolidin-2-ylidene) phosphoramidate linkage
  modified_base order(6..16,17..27): mod_base = OTHER; note = phosphorothioate
  modified_base 16^17: mod_base = OTHER; note = methylphosphonate
  modified_base order(3,19): mod_base = m5u
  modified_base 5: mod_base = m5c
ggtgctccacctggcacgtatatctctg

SEQ ID NO: 25 - DNA, 26 bp, synthetic construct
  source 1..26: mol_type = other DNA
  misc_feature 14: note = DNA
  misc_feature order(1..13,15..26): note = RNA
  modified_base order(1..3,12,17): mod_base = OTHER; note = 2&apos;-O-methoxyethyl
  modified_base order(4..5,15,24..26): mod_base = OTHER; note = 2&apos;-O-methyl
  modified_base order(6..11,16,18..23): mod_base = OTHER; note = 2&apos;-fluoro
  modified_base order(1^2,25^26): mod_base = OTHER; note = (1,3-dimethylimidazolidin-2-ylidene) phosphoramidate linkage
  modified_base order(4..12,13^14,16..25): mod_base = OTHER; note = phosphorothioate
  modified_base 14^15: mod_base = OTHER; note = methylphosphonate
  modified_base order(1,17): mod_base = m5u
  modified_base 3: mod_base = m5c
  modified_base 13: mod_base = OTHER; note = 6-amino-5-nitro-3-yl-2(1H)-pyridone nucleobase
tgctccacctggnacgtatatctctg

SEQ ID NO: 26 - DNA, 26 bp, synthetic construct
  source 1..26: mol_type = other DNA
  misc_feature 14: note = DNA
  misc_feature order(1..13,15..26): note = RNA
  modified_base order(1..3,12,17): mod_base = OTHER; note = 2&apos;-O-methoxyethyl
  modified_base order(4..6,8,10,15,19,21,23..26): mod_base = OTHER; note = 2&apos;-O-methyl
  modified_base order(7,9,11,16,18,20,22): mod_base = OTHER; note = 2&apos;-fluoro
  modified_base order(1^2,25^26): mod_base = OTHER; note = (1,3-dimethylimidazolidin-2-ylidene) phosphoramidate linkage
  modified_base order(4..12,13^14,16..25): mod_base = OTHER; note = phosphorothioate
  modified_base 14^15: mod_base = OTHER; note = methylphosphonate
  modified_base order(1,17): mod_base = m5u
  modified_base 3: mod_base = m5c
  modified_base 13: mod_base = OTHER; note = 6-amino-5-nitro-3-yl-2(1H)-pyridone nucleobase
tgctccacctggnacgtatatctctg

SEQ ID NO: 27 - DNA, 26 bp, synthetic construct
  source 1..26: mol_type = other DNA
  misc_feature 14: note = DNA
  misc_feature order(1..13,15..26): note = RNA
  modified_base order(1..3,12,17): mod_base = OTHER; note = 2&apos;-O-methoxyethyl
  modified_base order(4..5,15,24..26): mod_base = OTHER; note = 2&apos;-O-methyl
  modified_base order(6..11,16,18..23): mod_base = OTHER; note = 2&apos;-fluoro
  modified_base 13: mod_base = OTHER; note = 2&apos;,2&apos;-difluoro
  modified_base order(1^2,25^26): mod_base = OTHER; note = (1,3-dimethylimidazolidin-2-ylidene) phosphoramidate linkage
  modified_base order(4..12,13^14,16..25): mod_base = OTHER; note = phosphorothioate
  modified_base 14^15: mod_base = OTHER; note = methylphosphonate
  modified_base order(1,17): mod_base = m5u
  modified_base 3: mod_base = m5c
tgctccacctggcacgtatatctctg

SEQ ID NO: 28 - DNA, 26 bp, synthetic construct
  source 1..26: mol_type = other DNA
  misc_feature 14: note = DNA
  misc_feature order(1..13,15..26): note = RNA
  modified_base order(1..3,12,17): mod_base = OTHER; note = 2&apos;-O-methoxyethyl
  modified_base order(4..6,8,10,15,19,21,23..26): mod_base = OTHER; note = 2&apos;-O-methyl
  modified_base order(7,9,11,16,18,20,22): mod_base = OTHER; note = 2&apos;-fluoro
  modified_base 13: mod_base = OTHER; note = 2&apos;,2&apos;-difluoro
  modified_base order(1^2,25^26): mod_base = OTHER; note = (1,3-dimethylimidazolidin-2-ylidene) phosphoramidate linkage
  modified_base order(4..12,13^14,16..25): mod_base = OTHER; note = phosphorothioate
  modified_base 14^15: mod_base = OTHER; note = methylphosphonate
  modified_base order(1,17): mod_base = m5u
  modified_base 3: mod_base = m5c
tgctccacctggcacgtatatctctg

SEQ ID NO: 29 - DNA, 26 bp, synthetic construct
  source 1..26: mol_type = other DNA
  misc_feature 14: note = DNA
  misc_feature order(1..13,15..26): note = RNA
  modified_base order(1..3,12,17): mod_base = OTHER; note = 2&apos;-O-methoxyethyl
  modified_base order(4..5,15,24..26): mod_base = OTHER; note = 2&apos;-O-methyl
  modified_base order(6..11,16,18..23): mod_base = OTHER; note = 2&apos;-fluoro
  modified_base order(1^2,25^26): mod_base = OTHER; note = (1,3-dimethylimidazolidin-2-ylidene) phosphoramidate linkage
  modified_base order(4..14,15..25): mod_base = OTHER; note = phosphorothioate
  modified_base 14^15: mod_base = OTHER; note = methylphosphonate
  modified_base order(1,17): mod_base = m5u
  modified_base 3: mod_base = m5c
  modified_base 13: mod_base = OTHER; note = 6-amino-5-nitro-3-yl-2(1H)-pyridone nucleobase
tgctccacctggnacgtatatctctg

SEQ ID NO: 30 - DNA, 26 bp, synthetic construct
  source 1..26: mol_type = other DNA
  misc_feature 14: note = DNA
  misc_feature order(1..13,15..26): note = RNA
  modified_base order(1..3,12,17): mod_base = OTHER; note = 2&apos;-O-methoxyethyl
  modified_base order(4..6,8,10,15,19,21,23..26): mod_base = OTHER; note = 2&apos;-O-methyl
  modified_base order(7,9,11,16,18,20,22): mod_base = OTHER; note = 2&apos;-fluoro
  modified_base order(1^2,25^26): mod_base = OTHER; note = (1,3-dimethylimidazolidin-2-ylidene) phosphoramidate linkage
  modified_base order(4..14,15..25): mod_base = OTHER; note = phosphorothioate
  modified_base 14^15: mod_base = OTHER; note = methylphosphonate
  modified_base order(1,17): mod_base = m5u
  modified_base 3: mod_base = m5c
  modified_base 13: mod_base = OTHER; note = 6-amino-5-nitro-3-yl-2(1H)-pyridone nucleobase
tgctccacctggnacgtatatctctg

SEQ ID NO: 31 - DNA, 26 bp, synthetic construct
  source 1..26: mol_type = other DNA
  misc_feature 14: note = DNA
  misc_feature order(1..13,15..26): note = RNA
  modified_base order(1..3,12,17): mod_base = OTHER; note = 2&apos;-O-methoxyethyl
  modified_base order(4..5,15,24..26): mod_base = OTHER; note = 2&apos;-O-methyl
  modified_base order(6..11,16,18..23): mod_base = OTHER; note = 2&apos;-fluoro
  modified_base 13: mod_base = OTHER; note = 2&apos;,2&apos;-difluoro
  modified_base order(1^2,25^26): mod_base = OTHER; note = (1,3-dimethylimidazolidin-2-ylidene) phosphoramidate linkage
  modified_base order(4..14,15..25): mod_base = OTHER; note = phosphorothioate
  modified_base 14^15: mod_base = OTHER; note = methylphosphonate
  modified_base order(1,17): mod_base = m5u
  modified_base 3: mod_base = m5c
tgctccacctggcacgtatatctctg

SEQ ID NO: 32 - DNA, 26 bp, synthetic construct
  source 1..26: mol_type = other DNA
  misc_feature 14: note = DNA
  misc_feature order(1..13,15..26): note = RNA
  modified_base order(1..3,12,17): mod_base = OTHER; note = 2&apos;-O-methoxyethyl
  modified_base order(4..6,8,10,15,19,21,23..26): mod_base = OTHER; note = 2&apos;-O-methyl
  modified_base order(7,9,11,16,18,20,22): mod_base = OTHER; note = 2&apos;-fluoro
  modified_base 13: mod_base = OTHER; note = 2&apos;,2&apos;-difluoro
  modified_base order(1^2,25^26): mod_base = OTHER; note = (1,3-dimethylimidazolidin-2-ylidene) phosphoramidate linkage
  modified_base order(4..14,15..25): mod_base = OTHER; note = phosphorothioate
  modified_base 14^15: mod_base = OTHER; note = methylphosphonate
  modified_base order(1,17): mod_base = m5u
  modified_base 3: mod_base = m5c
tgctccacctggcacgtatatctctg

SEQ ID NO: 33 - DNA, 30 bp, synthetic construct
  source 1..30: mol_type = other DNA
  misc_feature 20: note = DNA
  misc_feature order(1..19,21..30): note = RNA
  modified_base order(1..5,18,23): mod_base = OTHER; note = 2&apos;-O-methoxyethyl
  modified_base order(6..11,21,30): mod_base = OTHER; note = 2&apos;-O-methyl
  modified_base order(12..17,22,24..29): mod_base = OTHER; note = 2&apos;-fluoro
  modified_base order(1^2,29^30): mod_base = OTHER; note = (1,3-dimethylimidazolidin-2-ylidene) phosphoramidate linkage
  modified_base order(6..18,19^20,22..29): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase
cctgggtgctccacctggnacgtatatctc

SEQ ID NO: 34 - DNA, 30 bp, synthetic construct; identical to SEQ ID NO: 33
  source 1..30: mol_type = other DNA
  misc_feature 20: note = DNA
  misc_feature order(1..19,21..30): note = RNA
  modified_base order(1..5,18,23): mod_base = OTHER; note = 2&apos;-O-methoxyethyl
  modified_base order(6..12,14,16,21,25,27,29..30): mod_base = OTHER; note = 2&apos;-O-methyl
  modified_base order(13,15,17,22,24,26,28): mod_base = OTHER; note = 2&apos;-fluoro
  modified_base order(1^2,29^30): mod_base = OTHER; note = (1,3-dimethylimidazolidin-2-ylidene) phosphoramidate linkage
  modified_base order(6..18,19^20,22..29): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase

SEQ ID NO: 35 - DNA, 30 bp, synthetic construct
  source 1..30: mol_type = other DNA
  misc_feature 20: note = DNA
  misc_feature order(1..19,21..30): note = RNA
  modified_base order(1..5,18,23): mod_base = OTHER; note = 2&apos;-O-methoxyethyl
  modified_base order(6..11,21,30): mod_base = OTHER; note = 2&apos;-O-methyl
  modified_base order(12..17,22,24..29): mod_base = OTHER; note = 2&apos;-fluoro
  modified_base 19: mod_base = OTHER; note = 2&apos;,2&apos;-difluoro
  modified_base order(1^2,29^30): mod_base = OTHER; note = (1,3-dimethylimidazolidin-2-ylidene) phosphoramidate linkage
  modified_base order(6..18,19^20,22..29): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
cctgggtgctccacctggcacgtatatctc

SEQ ID NO: 36 - DNA, 30 bp, synthetic construct; identical to SEQ ID NO: 35
  source 1..30: mol_type = other DNA
  misc_feature 20: note = DNA
  misc_feature order(1..19,21..30): note = RNA
  modified_base order(1..5,18,23): mod_base = OTHER; note = 2&apos;-O-methoxyethyl
  modified_base order(6..12,14,16,21,25,27,29..30): mod_base = OTHER; note = 2&apos;-O-methyl
  modified_base order(13,15,17,22,24,26,28): mod_base = OTHER; note = 2&apos;-fluoro
  modified_base 19: mod_base = OTHER; note = 2&apos;,2&apos;-difluoro
  modified_base order(1^2,29^30): mod_base = OTHER; note = (1,3-dimethylimidazolidin-2-ylidene) phosphoramidate linkage
  modified_base order(6..18,19^20,22..29): mod_base = OTHER; note = phosphorothioate
  modified_base 20^21: mod_base = OTHER; note = methylphosphonate
  modified_base 1..2: mod_base = m5c
  modified_base order(3,23): mod_base = m5u

SEQ ID NO: 37 - DNA, 30 bp, synthetic construct; identical to SEQ ID NO: 33
  source 1..30: mol_type = other DNA
  misc_feature 20: note = DNA
  misc_feature order(1..19,21..30): note = RNA
  modified_base order(1..5,18,23): mod_base = OTHER; note = 2&apos;-O-methoxyethyl
  modified_base order(6..11,21,30): mod_base = OTHER; note = 2&apos;-O-methyl
  modified_base order(12..17,22,24..29): mod_base = OTHER; note = 2&apos;-fluoro
  modified_base order(1^2,29^30): mod_base = OTHER; note = (1,3-dimethylimidazolidin-2-ylidene) phosphoramidate linkage
  modified_base order(6..20,21..29): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase

SEQ ID NO: 38 - DNA, 30 bp, synthetic construct; identical to SEQ ID NO: 33
  source 1..30: mol_type = other DNA
  misc_feature 20: note = DNA
  misc_feature order(1..19,21..30): note = RNA
  modified_base order(1..5,18,23): mod_base = OTHER; note = 2&apos;-O-methoxyethyl
  modified_base order(6..12,14,16,21,25,27,29..30): mod_base = OTHER; note = 2&apos;-O-methyl
  modified_base order(13,15,17,22,24,26,28): mod_base = OTHER; note = 2&apos;-fluoro
  modified_base order(1^2,29^30): mod_base = OTHER; note = (1,3-dimethylimidazolidin-2-ylidene) phosphoramidate linkage
  modified_base order(6..20,21..29): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase

SEQ ID NO: 39 - DNA, 30 bp, synthetic construct; identical to SEQ ID NO: 35
  source 1..30: mol_type = other DNA
  misc_feature 20: note = DNA
  misc_feature order(1..19,21..30): note = RNA
  modified_base order(1..5,18,23): mod_base = OTHER; note = 2&apos;-O-methoxyethyl
  modified_base order(6..11,21,30): mod_base = OTHER; note = 2&apos;-O-methyl
  modified_base order(12..17,22,24..29): mod_base = OTHER; note = 2&apos;-fluoro
  modified_base 19: mod_base = OTHER; note = 2&apos;,2&apos;-difluoro
  modified_base order(1^2,29^30): mod_base = OTHER; note = (1,3-dimethylimidazolidin-2-ylidene) phosphoramidate linkage
  modified_base order(6..20,21..29): mod_base = OTHER; note = phosphorothioate
  modified_base 20^21: mod_base = OTHER; note = methylphosphonate
  modified_base 1..2: mod_base = m5c
  modified_base order(3,23): mod_base = m5u

SEQ ID NO: 40 - DNA, 30 bp, synthetic construct; identical to SEQ ID NO: 35
  source 1..30: mol_type = other DNA
  misc_feature 20: note = DNA
  misc_feature order(1..19,21..30): note = RNA
  modified_base order(1..5,18,23): mod_base = OTHER; note = 2&apos;-O-methoxyethyl
  modified_base order(6..12,14,16,21,25,27,29..30): mod_base = OTHER; note = 2&apos;-O-methyl
  modified_base order(13,15,17,22,24,26,28): mod_base = OTHER; note = 2&apos;-fluoro
  modified_base 19: mod_base = OTHER; note = 2&apos;,2&apos;-difluoro
  modified_base order(1^2,29^30): mod_base = OTHER; note = (1,3-dimethylimidazolidin-2-ylidene) phosphoramidate linkage
  modified_base order(6..20,21..29): mod_base = OTHER; note = phosphorothioate
  modified_base 20^21: mod_base = OTHER; note = methylphosphonate
  modified_base 1..2: mod_base = m5c
  modified_base order(3,23): mod_base = m5u

SEQ ID NO: 41 - DNA, 28 bp, synthetic construct
  source 1..28: mol_type = other DNA
  misc_feature 20: note = DNA
  misc_feature order(1..19,21..28): note = RNA
  modified_base order(1..5,18,23): mod_base = OTHER; note = 2&apos;-O-methoxyethyl
  modified_base order(6..11,21): mod_base = OTHER; note = 2&apos;-O-methyl
  modified_base order(12..17,22,24..28): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(6..18,19^20,22..27): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase
cctgggtgctccacctggnacgtatatc

SEQ ID NO: 42 - DNA, 28 bp, synthetic construct
  source 1..28: mol_type = other DNA
  misc_feature 20: note = DNA
  misc_feature order(1..19,21..28): note = RNA
  modified_base order(1..5,18,23): mod_base = OTHER; note = 2&apos;-O-methoxyethyl
  modified_base order(6..12,14,16,21,25,27): mod_base = OTHER; note = 2&apos;-O-methyl
  modified_base order(13,15,17,22,24,26,28): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(6..18,19^20,22..27): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase
cctgggtgctccacctggnacgtatatc

SEQ ID NO: 43 - DNA, 28 bp, synthetic construct
  source 1..28: mol_type = other DNA
  misc_feature 20: note = DNA
  misc_feature order(1..19,21..28): note = RNA
  modified_base order(1..5,18,23): mod_base = OTHER; note = 2&apos;-O-methoxyethyl
  modified_base order(6..11,21): mod_base = OTHER; note = 2&apos;-O-methyl
  modified_base order(12..17,22,24..28): mod_base = OTHER; note = 2&apos;-fluoro
  modified_base 19: mod_base = OTHER; note = 2&apos;,2&apos;-difluoro
  modified_base order(1^2,27^28): mod_base = OTHER; note = (1,3-dimethylimidazolidin-2-ylidene) phosphoramidate linkage
  modified_base order(6..18,19^20,22..27): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
cctgggtgctccacctggcacgtatatc

SEQ ID NO: 44 - DNA, 28 bp, synthetic construct
  source 1..28: mol_type = other DNA
  misc_feature 20: note = DNA
  misc_feature order(1..19,21..28): note = RNA
  modified_base order(1..5,18,23): mod_base = OTHER; note = 2&apos;-O-methoxyethyl
  modified_base order(6..12,14,16,21,25,27): mod_base = OTHER; note = 2&apos;-O-methyl
  modified_base order(13,15,17,22,24,26,28): mod_base = OTHER; note = 2&apos;-fluoro
  modified_base 19: mod_base = OTHER; note = 2&apos;,2&apos;-difluoro
  modified_base order(1^2,27^28): mod_base = OTHER; note = (1,3-dimethylimidazolidin-2-ylidene) phosphoramidate linkage
  modified_base order(6..18,19^20,22..27): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
cctgggtgctccacctggcacgtatatc

SEQ ID NO: 45 - DNA, 28 bp, synthetic construct
  source 1..28: mol_type = other DNA
  misc_feature 20: note = DNA
  misc_feature order(1..19,21..28): note = RNA
  modified_base order(1..5,18,23): mod_base = OTHER; note = 2&apos;-O-methoxyethyl
  modified_base order(6..11,21): mod_base = OTHER; note = 2&apos;-O-methyl
  modified_base order(12..17,22,24..28): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(6..20,21..27): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase
cctgggtgctccacctggnacgtatatc

SEQ ID NO: 46 - DNA, 28 bp, synthetic construct
  source 1..28: mol_type = other DNA
  misc_feature 20: note = DNA
  misc_feature order(1..19,21..28): note = RNA
  modified_base order(1..5,18,23): mod_base = OTHER; note = 2&apos;-O-methoxyethyl
  modified_base order(6..12,14,16,21,25,27): mod_base = OTHER; note = 2&apos;-O-methyl
  modified_base order(13,15,17,22,24,26,28): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(6..20,21..27): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
  modified_base 19: mod_base = OTHER; note = 6-amino-5-nitro-3-yl-2(1H)-pyridone nucleobase
cctgggtgctccacctggnacgtatatc

SEQ ID NO: 47 - DNA, 28 bp, synthetic construct
  source 1..28: mol_type = other DNA
  misc_feature 20: note = DNA
  misc_feature order(1..19,21..28): note = RNA
  modified_base order(1..5,18,23): mod_base = OTHER; note = 2&apos;-O-methoxyethyl
  modified_base order(6..11,21): mod_base = OTHER; note = 2&apos;-O-methyl
  modified_base order(12..17,22,24..28): mod_base = OTHER; note = 2&apos;-fluoro
  modified_base 19: mod_base = OTHER; note = 2&apos;,2&apos;-difluoro
  modified_base order(1^2,27^28): mod_base = OTHER; note = (1,3-dimethylimidazolidin-2-ylidene) phosphoramidate linkage
  modified_base order(6..20,21..27): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
cctgggtgctccacctggcacgtatatc

SEQ ID NO: 48 - DNA, 28 bp, synthetic construct
  source 1..28: mol_type = other DNA
  misc_feature 20: note = DNA
  misc_feature order(1..19,21..28): note = RNA
  modified_base order(1..5,18,23): mod_base = OTHER; note = 2&apos;-O-methoxyethyl
  modified_base order(6..12,14,16,21,25,27): mod_base = OTHER; note = 2&apos;-O-methyl
  modified_base order(13,15,17,22,24,26,28): mod_base = OTHER; note = 2&apos;-fluoro
  modified_base 19: mod_base = OTHER; note = 2&apos;,2&apos;-difluoro
  modified_base order(1^2,27^28): mod_base = OTHER; note = (1,3-dimethylimidazolidin-2-ylidene) phosphoramidate linkage
  modified_base order(6..20,21..27): mod_base = OTHER; note = phosphorothioate
  modified_base 20^21: mod_base = OTHER; note = methylphosphonate
  modified_base 1..2: mod_base = m5c
  modified_base order(3,23): mod_base = m5u
cctgggtgctccacctggcacgtatatc

SEQ ID NO: 49 - DNA, 30 bp, synthetic construct
  source 1..30: mol_type = other DNA
  misc_feature 25..26: note = DNA
  misc_feature order(1..24,27..30): note = RNA
  modified_base order(1..15,24): mod_base = OTHER; note = 2&apos;-fluoro
  modified_base order(16..23,27..30): mod_base = OTHER; note = 2&apos;-O-methyl
  modified_base order(1^2,16^17,26^27,29^30): mod_base = OTHER; note = (1,3-dimethylimidazolidin-2-ylidene) phosphoramidate linkage
  modified_base order(2..16,17..26,27..29): mod_base = OTHER; note = phosphorothioate
tccaggcctgggtgctccacctggcacgta

SEQ ID NO: 50 - DNA, 30 bp, synthetic construct
  source 1..30: mol_type = other DNA
  misc_feature 26: note = DNA
  misc_feature order(1..25,27..30): note = RNA
  modified_base order(1..15,24): mod_base = OTHER; note = 2&apos;-fluoro
  modified_base order(16..23,27..30): mod_base = OTHER; note = 2&apos;-O-methyl
  modified_base order(1^2,16^17,26^27,29^30): mod_base = OTHER; note = (1,3-dimethylimidazolidin-2-ylidene) phosphoramidate linkage
  modified_base order(2..16,17..26,27..29): mod_base = OTHER; note = phosphorothioate
  modified_base 25: mod_base = OTHER; note = 6-amino-5-nitro-3-yl-2(1H)-pyridone nucleobase
tccaggcctgggtgctccacctggnacgta

SEQ ID NO: 51 - DNA, 30 bp, synthetic construct; identical to SEQ ID NO: 49
  source 1..30: mol_type = other DNA
  misc_feature 26: note = DNA
  misc_feature order(1..25,27..30): note = RNA
  modified_base order(1..15,24): mod_base = OTHER; note = 2&apos;-fluoro
  modified_base order(16..23,27..30): mod_base = OTHER; note = 2&apos;-O-methyl
  modified_base 25: mod_base = OTHER; note = 2&apos;,2&apos;-difluoro
  modified_base order(1^2,16^17,26^27,29^30): mod_base = OTHER; note = (1,3-dimethylimidazolidin-2-ylidene) phosphoramidate linkage
  modified_base order(2..16,17..26,27..29): mod_base = OTHER; note = phosphorothioate

SEQ ID NO: 52 - RNA, 38 nt, Homo sapiens
  source 1..38: mol_type = transcribed RNA
cagagatatacgtaccaggtggagcacccaggcctgga

SEQ ID NO: 53 - DNA, 20 bp, synthetic construct
  source 1..20: mol_type = other DNA
ctcgacgcaagccataacac

SEQ ID NO: 54 - DNA, 20 bp, synthetic construct
  source 1..20: mol_type = other DNA
tggaccgactggaaacgtag

SEQ ID NO: 55 - DNA, 272 bp, synthetic construct
  source 1..272: mol_type = other DNA
tctggctcgacgcaagccataacactaatacgactcactatagggagttcgaacctaaagacgtattgcccaatggggatgggacctaccagggctggataaccttggctgtaccccctggggaagagcagagatatacgtaccaggtggagcacccaggcctggatcagcccctcattgtgatctgggagccctcaccgtctggcaccctagtcattggagtcatcagtggaattgctgtttttgtcgtacgtttccagtcggtccacgtt

SEQ ID NO: 56 - DNA, 19 bp, synthetic construct
  source 1..19: mol_type = other DNA
ggcgcttctcctcctgatg

SEQ ID NO: 57 - DNA, 23 bp, synthetic construct
  source 1..23: mol_type = other DNA
tgctgcgttcacactctctgcac

SEQ ID NO: 58 - DNA, 20 bp, synthetic construct
  source 1..20: mol_type = other DNA
ccacgtagcccaaagcttca

SEQ ID NO: 59 - DNA, 20 bp, synthetic construct
  source 1..20: mol_type = other DNA
cgtattgcccaatggggatg

SEQ ID NO: 60 - DNA, 23 bp, synthetic construct
  source 1..23: mol_type = other DNA
  modified_base 14..15: mod_base = OTHER; note = locked nucleic acid
cagagatatacgtgccaggtgga

SEQ ID NO: 61 - DNA, 23 bp, synthetic construct
  source 1..23: mol_type = other DNA
  modified_base 13..15: mod_base = OTHER; note = locked nucleic acid
cagagatatacgtaccaggtgga

SEQ ID NO: 62 - DNA, 20 bp, synthetic construct
  source 1..20: mol_type = other DNA
actccaatgactagggtgcc

SEQ ID NO: 63 - DNA, 20 bp, synthetic construct
  source 1..20: mol_type = other DNA
tgggaaaggcacaagattcg

SEQ ID NO: 64 - DNA, 20 bp, synthetic construct
  source 1..20: mol_type = other DNA
  modified_base 12: mod_base = OTHER; note = locked nucleic acid
ggaccaacaagagccctcac

SEQ ID NO: 65 - DNA, 20 bp, synthetic construct
  source 1..20: mol_type = other DNA
cagctaagacgtagtgcccc

SEQ ID NO: 66 - DNA, 32 bp, synthetic construct; identical to SEQ ID NO: 1
  source 1..32: mol_type = other DNA
  misc_feature 20: note = DNA
  misc_feature order(1..19,21..32): note = RNA
  modified_base order(1..8,18,23): mod_base = OTHER; note = 2&apos;-O-methoxyethyl
  modified_base order(9..12,14,16,21,25,27,29..32): mod_base = OTHER; note = 2&apos;-O-methyl
  modified_base order(13,15,17,22,24,26,28): mod_base = OTHER; note = 2&apos;-fluoro
  modified_base order(9..18,19^20,22..32): mod_base = OTHER; note = phosphorothioate
  modified_base 20^21: mod_base = OTHER; note = methylphosphonate
  modified_base 1..2: mod_base = m5c
  modified_base order(3,7,23): mod_base = m5u
  modified_base 19: mod_base = OTHER; note = 6-amino-5-nitro-3-yl-2(1H)-pyridone nucleobase

SEQ ID NO: 67 - DNA, 31 bp, synthetic construct
  source 1..31: mol_type = other DNA
  misc_feature 19: note = DNA
  misc_feature order(1..18,20..31): note = RNA
  modified_base order(1..7,17,22): mod_base = OTHER; note = 2&apos;-O-methoxyethyl
  modified_base order(8..11,13,15,20,24,26,28..31): mod_base = OTHER; note = 2&apos;-O-methyl
  modified_base order(12,14,16,21,23,25,27): mod_base = OTHER; note = 2&apos;-fluoro
  modified_base order(8..17,18^19,21..31): mod_base = OTHER; note = phosphorothioate
  modified_base 19^20: mod_base = OTHER; note = methylphosphonate
  modified_base 1: mod_base = m5c
  modified_base order(2,6,22): mod_base = m5u
  modified_base 18: mod_base = OTHER; note = 6-amino-5-nitro-3-yl-2(1H)-pyridone nucleobase
ctgggtgctccacctggnacgtatatctctg

SEQ ID NO: 68 - DNA, 30 bp, synthetic construct; identical to SEQ ID NO: 9
  source 1..30: mol_type = other DNA
  misc_feature 18: note = DNA
  misc_feature order(1..17,19..30): note = RNA
  modified_base order(1..6,16,21): mod_base = OTHER; note = 2&apos;-O-methoxyethyl
  modified_base order(7..10,12,14,19,23,25,27..30): mod_base = OTHER; note = 2&apos;-O-methyl
  modified_base order(11,13,15,20,22,24,26): mod_base = OTHER; note = 2&apos;-fluoro
  modified_base order(7..16,17^18,20..30): mod_base = OTHER; note = phosphorothioate
  modified_base 18^19: mod_base = OTHER; note = methylphosphonate
  modified_base order(1,5,21): mod_base = m5u
  modified_base 17: mod_base = OTHER; note = 6-amino-5-nitro-3-yl-2(1H)-pyridone nucleobase

SEQ ID NO: 69 - DNA, 29 bp, synthetic construct
  source 1..29: mol_type = other DNA
  misc_feature 17: note = DNA
  misc_feature order(1..16,18..29): note = RNA
  modified_base order(1..5,15,20): mod_base = OTHER; note = 2&apos;-O-methoxyethyl
  modified_base order(6..9,11,13,18,22,24,26..29): mod_base = OTHER; note = 2&apos;-O-methyl
  modified_base order(10,12,14,19,21,23,25): mod_base = OTHER; note = 2&apos;-fluoro
  modified_base order(6..15,16^17,19..29): mod_base = OTHER; note = phosphorothioate
  modified_base 17^18: mod_base = OTHER; note = methylphosphonate
  modified_base order(4,20): mod_base = m5u
  modified_base 16: mod_base = OTHER; note = 6-amino-5-nitro-3-yl-2(1H)-pyridone nucleobase
gggtgctccacctggnacgtatatctctg

SEQ ID NO: 70 - DNA, 28 bp, synthetic construct
  source 1..28: mol_type = other DNA
  misc_feature 16: note = DNA
  misc_feature order(1..15,17..28): note = RNA
  modified_base order(1..4,14,19): mod_base = OTHER; note = 2&apos;-O-methoxyethyl
  modified_base order(5..8,10,12,17,21,23,25..28): mod_base = OTHER; note = 2&apos;-O-methyl
  modified_base order(9,11,13,18,20,22,24): mod_base = OTHER; note = 2&apos;-fluoro
  modified_base order(5..14,15^16,18..28): mod_base = OTHER; note = phosphorothioate
  modified_base 16^17: mod_base = OTHER; note = methylphosphonate
  modified_base order(3,19): mod_base = m5u
  modified_base 15: mod_base = OTHER; note = 6-amino-5-nitro-3-yl-2(1H)-pyridone nucleobase
ggtgctccacctggnacgtatatctctg

SEQ ID NO: 71 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..7,9,11,16,20,22,24..27): mod_base = OTHER; note = 2&apos;-O-methyl
  modified_base order(8,10,12,17,19,21,23): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 72 - DNA, 25 bp, synthetic construct
  source 1..25: mol_type = other DNA
  misc_feature 13: note = DNA
  misc_feature order(1..12,14..25): note = RNA
  modified_base order(1,11,16): mod_base = OTHER; note = 2&apos;-O-methoxyethyl
  modified_base order(2..5,7,9,14,18,20,22..25): mod_base = OTHER; note = 2&apos;-O-methyl
  modified_base order(6,8,10,15,17,19,21): mod_base = OTHER; note = 2&apos;-fluoro
  modified_base order(2..11,12^13,15..25): mod_base = OTHER; note = phosphorothioate
  modified_base 13^14: mod_base = OTHER; note = methylphosphonate
  modified_base 12: mod_base = OTHER; note = 6-amino-5-nitro-3-yl-2(1H)-pyridone nucleobase
  modified_base 16: mod_base = m5u
gctccacctggnacgtatatctctg

SEQ ID NO: 73 - DNA, 23 bp, synthetic construct
  source 1..23: mol_type = other DNA
  misc_feature 11: note = DNA
  misc_feature order(1..10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1..9,10^11,13..23): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 74 - DNA, 32 bp, synthetic construct
  source 1..32: mol_type = other DNA
  misc_feature 12: note = DNA
  misc_feature order(1..11,13..32): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..32): mod_base = OTHER; note = 2&apos;-O-methoxyethyl
  modified_base order(1..10,11^12,14..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30..32): mod_base = m5c
ctccacctggnacgtatatctctgctcttccc

SEQ ID NO: 75 - DNA, 31 bp, synthetic construct
  source 1..31: mol_type = other DNA
  misc_feature 12: note = DNA
  misc_feature order(1..11,13..31): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..31): mod_base = OTHER; note = 2&apos;-O-methoxyethyl
  modified_base order(1..10,11^12,14..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30..31): mod_base = m5c
ctccacctggnacgtatatctctgctcttcc

SEQ ID NO: 76 - DNA, 30 bp, synthetic construct
  source 1..30: mol_type = other DNA
  misc_feature 12: note = DNA
  misc_feature order(1..11,13..30): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..30): mod_base = OTHER; note = 2&apos;-O-methoxyethyl
  modified_base order(1..10,11^12,14..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30): mod_base = m5c
ctccacctggnacgtatatctctgctcttc

SEQ ID NO: 77 - DNA, 29 bp, synthetic construct
  source 1..29: mol_type = other DNA
  misc_feature 12: note = DNA
  misc_feature order(1..11,13..29): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..29): mod_base = OTHER; note = 2&apos;-O-methoxyethyl
  modified_base order(1..10,11^12,14..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27): mod_base = m5c
ctccacctggnacgtatatctctgctctt

SEQ ID NO: 78 - DNA, 28 bp, synthetic construct
  source 1..28: mol_type = other DNA
  misc_feature 12: note = DNA
  misc_feature order(1..11,13..28): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..28): mod_base = OTHER; note = 2&apos;-O-methoxyethyl
  modified_base order(1..10,11^12,14..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28): mod_base = m5u
  modified_base order(25,27): mod_base = m5c
ctccacctggnacgtatatctctgctct

SEQ ID NO: 79 - DNA, 27 bp, synthetic construct
  source 1..27: mol_type = other DNA
  misc_feature 12: note = DNA
  misc_feature order(1..11,13..27): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..27): mod_base = OTHER; note = 2&apos;-O-methoxyethyl
  modified_base order(1..10,11^12,14..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26): mod_base = m5u
  modified_base order(25,27): mod_base = m5c
ctccacctggnacgtatatctctgctc

SEQ ID NO: 80 - DNA, 28 bp, synthetic construct
  source 1..28: mol_type = other DNA
  misc_feature 17: note = DNA
  misc_feature order(1..16,18..28): note = RNA
  modified_base order(1..5,15,20): mod_base = OTHER; note = 2&apos;-O-methoxyethyl
  modified_base order(6..9,11,13,18,22,24,26..28): mod_base = OTHER; note = 2&apos;-O-methyl
  modified_base order(10,12,14,19,21,23,25): mod_base = OTHER; note = 2&apos;-fluoro
  modified_base order(6..15,16^17,19..28): mod_base = OTHER; note = phosphorothioate
  modified_base 17^18: mod_base = OTHER; note = methylphosphonate
  modified_base order(4,20): mod_base = m5u
  modified_base 16: mod_base = OTHER; note = 6-amino-5-nitro-3-yl-2(1H)-pyridone nucleobase
gggtgctccacctggnacgtatatctct

SEQ ID NO: 81 - DNA, 27 bp, synthetic construct
  source 1..27: mol_type = other DNA
  misc_feature 17: note = DNA
  misc_feature order(1..16,18..27): note = RNA
  modified_base order(1..5,15,20): mod_base = OTHER; note = 2&apos;-O-methoxyethyl
  modified_base order(6..9,11,13,18,22,24,26..27): mod_base = OTHER; note = 2&apos;-O-methyl
  modified_base order(10,12,14,19,21,23,25): mod_base = OTHER; note = 2&apos;-fluoro
  modified_base order(6..15,16^17,19..27): mod_base = OTHER; note = phosphorothioate
  modified_base 17^18: mod_base = OTHER; note = methylphosphonate
  modified_base order(4,20): mod_base = m5u
  modified_base 16: mod_base = OTHER; note = 6-amino-5-nitro-3-yl-2(1H)-pyridone nucleobase
gggtgctccacctggnacgtatatctc

SEQ ID NO: 82 - DNA, 26 bp, synthetic construct
  source 1..26: mol_type = other DNA
  misc_feature 17: note = DNA
  misc_feature order(1..16,18..26): note = RNA
  modified_base order(1..5,15,20): mod_base = OTHER; note = 2&apos;-O-methoxyethyl
  modified_base order(6..9,11,13,18,22,24,26): mod_base = OTHER; note = 2&apos;-O-methyl
  modified_base order(10,12,14,19,21,23,25): mod_base = OTHER; note = 2&apos;-fluoro
  modified_base order(6..15,16^17,19..26): mod_base = OTHER; note = phosphorothioate
  modified_base 17^18: mod_base = OTHER; note = methylphosphonate
  modified_base order(4,20): mod_base = m5u
  modified_base 16: mod_base = OTHER; note = 6-amino-5-nitro-3-yl-2(1H)-pyridone nucleobase
gggtgctccacctggnacgtatatct

SEQ ID NO: 83 - DNA, 25 bp, synthetic construct
  source 1..25: mol_type = other DNA
  misc_feature 17: note = DNA
  misc_feature order(1..16,18..25): note = RNA
  modified_base order(1..5,15,20): mod_base = OTHER; note = 2&apos;-O-methoxyethyl
  modified_base order(6..9,11,13,18,22,24): mod_base = OTHER; note = 2&apos;-O-methyl
  modified_base order(10,12,14,19,21,23,25): mod_base = OTHER; note = 2&apos;-fluoro
  modified_base order(6..15,16^17,19..25): mod_base = OTHER; note = phosphorothioate
  modified_base 17^18: mod_base = OTHER; note = methylphosphonate
  modified_base order(4,20): mod_base = m5u
  modified_base 16: mod_base = OTHER; note = 6-amino-5-nitro-3-yl-2(1H)-pyridone nucleobase
gggtgctccacctggnacgtatatc

SEQ ID NO: 84 - DNA, 24 bp, synthetic construct
  source 1..24: mol_type = other DNA
  misc_feature 17: note = DNA
  misc_feature order(1..16,18..24): note = RNA
  modified_base order(1..5,15,20): mod_base = OTHER; note = 2&apos;-O-methoxyethyl
  modified_base order(6..9,11,13,18,22,24): mod_base = OTHER; note = 2&apos;-O-methyl
  modified_base order(10,12,14,19,21,23): mod_base = OTHER; note = 2&apos;-fluoro
  modified_base order(6..15,16^17,19..24): mod_base = OTHER; note = phosphorothioate
  modified_base 17^18: mod_base = OTHER; note = methylphosphonate
  modified_base order(4,20): mod_base = m5u
  modified_base 16: mod_base = OTHER; note = 6-amino-5-nitro-3-yl-2(1H)-pyridone nucleobase
gggtgctccacctggnacgtatat

SEQ ID NO: 85 - DNA, 30 bp, synthetic construct
  source 1..30: mol_type = other DNA
  misc_feature order(23,25): note = DNA
  misc_feature order(1..22,24,26..30): note = RNA
  modified_base order(1..3,6..7,10,12,16,20..21,26,28..30): mod_base = OTHER; note = 2&apos;-O-methyl
  modified_base order(4..5,8..9,11,13..15,17..19,22,27): mod_base = OTHER; note = 2&apos;-fluoro
  modified_base order(1^2,11^12,14^15,25^26,29^30): mod_base = OTHER; note = (1,3-dimethylimidazolidin-2-ylidene) phosphoramidate linkage
  modified_base order(2..6,7..9,10^11,13^14,16..20,22..25,27..29): mod_base = OTHER; note = phosphorothioate
  modified_base 24: mod_base = OTHER; note = 6-amino-5-nitro-3-yl-2(1H)-pyridone nucleobase
ccaggcctgggtgctccacctggnacgtat

SEQ ID NO: 86 - DNA, 30 bp, synthetic construct; identical to SEQ ID NO: 85
  source 1..30: mol_type = other DNA
  misc_feature 25: note = DNA
  misc_feature order(1..24,26..30): note = RNA
  modified_base order(1..3,6..7,10,12,16,20..21,26,29..30): mod_base = OTHER; note = 2&apos;-O-methyl
  modified_base order(4..5,8..9,11,13..15,17..19,22,27): mod_base = OTHER; note = 2&apos;-fluoro
  modified_base order(23,28): mod_base = OTHER; note = 2&apos;-O-methoxyethyl
  modified_base order(1^2,11^12,14^15,29^30): mod_base = OTHER; note = (1,3-dimethylimidazolidin-2-ylidene) phosphoramidate linkage
  modified_base order(2..6,7..9,10^11,13^14,16..20,22^23,24^25,27..29): mod_base = OTHER; note = phosphorothioate
  modified_base 25^26: mod_base = OTHER; note = methylphosphonate
  modified_base 24: mod_base = OTHER; note = 6-amino-5-nitro-3-yl-2(1H)-pyridone nucleobase
  modified_base 28: mod_base = m5u

SEQ ID NO: 87 - DNA, 30 bp, synthetic construct; identical to SEQ ID NO: 85
  source 1..30: mol_type = other DNA
  misc_feature order(23,25): note = DNA
  misc_feature order(1..22,24,26..30): note = RNA
  modified_base order(1..3,9,11..12,15,17,26,28..30): mod_base = OTHER; note = 2&apos;-O-methyl
  modified_base order(4..5,8,13..14,18..19,22,27): mod_base = OTHER; note = 2&apos;-fluoro
  modified_base order(6..7,10,16,20..21): mod_base = OTHER; note = 2&apos;-O-methoxyethyl
  modified_base order(1^2,11^12,14^15,25^26,29^30): mod_base = OTHER; note = (1,3-dimethylimidazolidin-2-ylidene) phosphoramidate linkage
  modified_base order(2..6,8^9,10^11,13^14,17..20,22..25,27..29): mod_base = OTHER; note = phosphorothioate
  modified_base order(6..7,16,20): mod_base = m5c
  modified_base 21: mod_base = m5u
  modified_base 24: mod_base = OTHER; note = 6-amino-5-nitro-3-yl-2(1H)-pyridone nucleobase

SEQ ID NO: 88 - DNA, 30 bp, synthetic construct; identical to SEQ ID NO: 85
  source 1..30: mol_type = other DNA
  misc_feature 25: note = DNA
  misc_feature order(1..24,26..30): note = RNA
  modified_base order(1..3,9,11..12,15,17,26,29..30): mod_base = OTHER; note = 2&apos;-O-methyl
  modified_base order(4..5,8,13..14,18..19,22,27): mod_base = OTHER; note = 2&apos;-fluoro
  modified_base order(6..7,10,16,20..21,23,28): mod_base = OTHER; note = 2&apos;-O-methoxyethyl
  modified_base order(1^2,11^12,14^15,29^30): mod_base = OTHER; note = (1,3-dimethylimidazolidin-2-ylidene) phosphoramidate linkage
  modified_base order(2..6,8^9,10^11,13^14,17..20,22^23,24^25,27..29): mod_base = OTHER; note = phosphorothioate
  modified_base 25^26: mod_base = OTHER; note = methylphosphonate
  modified_base order(6..7,16,20): mod_base = m5c
  modified_base order(21,28): mod_base = m5u
  modified_base 24: mod_base = OTHER; note = 6-amino-5-nitro-3-yl-2(1H)-pyridone nucleobase

SEQ ID NO: 89 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..12,17): mod_base = OTHER; note = 2&apos;-fluoro
  modified_base order(16,19..27): mod_base = OTHER; note = 2&apos;-O-methyl
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 90 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..12,16): mod_base = OTHER; note = 2&apos;-O-methyl
  modified_base order(17,19..27): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 91 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..6,16,19..27): mod_base = OTHER; note = 2&apos;-O-methyl
  modified_base order(7..12,17): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 92 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..12,16,25..27): mod_base = OTHER; note = 2&apos;-O-methyl
  modified_base order(17,19..24): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 93 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..5,7..9,11,16,20,22..24,26..27): mod_base = OTHER; note = 2&apos;-O-methyl
  modified_base order(6,10,12,17,19,21,25): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 94 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..6,16,25..27): mod_base = OTHER; note = 2&apos;-O-methyl
  modified_base order(7..12,17,19..24): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 95 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4,6,8,10,12,17,19,21,23,25,27): mod_base = OTHER; note = 2&apos;-fluoro
  modified_base order(5,7,9,11,16,20,22,24,26): mod_base = OTHER; note = 2&apos;-O-methyl
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 96 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4,6,8,10,12,17): mod_base = OTHER; note = 2&apos;-fluoro
  modified_base order(5,7,9,11,16,19..27): mod_base = OTHER; note = 2&apos;-O-methyl
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 97 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..12,16,20,22,24,26): mod_base = OTHER; note = 2&apos;-O-methyl
  modified_base order(17,19,21,23,25,27): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 98 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..5,7,9,11,16,20,22,24,26..27): mod_base = OTHER; note = 2&apos;-O-methyl
  modified_base order(6,8,10,12,17,19,21,23,25): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 99 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..9,11,16,20,22..27): mod_base = OTHER; note = 2&apos;-O-methyl
  modified_base order(10,12,17,19,21): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 100 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..6,8,10,12,16,19,21,23,25..27): mod_base = OTHER; note = 2&apos;-O-methyl
  modified_base order(7,9,11,17,20,22,24): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 101 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..6,9..10,16,21..22,25..27): mod_base = OTHER; note = 2&apos;-O-methyl
  modified_base order(7..8,11..12,17,19..20,23..24): mod_base = OTHER; note = 2&apos;-fluoro
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 102 - DNA, 27 bp, synthetic construct
  source 1..27: mol_type = other DNA
  misc_feature 15: note = DNA
  misc_feature order(1..14,16..27): note = RNA
  modified_base order(1..7,9,11,16,20,22,24..27): mod_base = OTHER; note = 2&apos;-O-methyl
  modified_base order(8,10,12,17,19,21,23): mod_base = OTHER; note = 2&apos;-fluoro
  modified_base order(13,18): mod_base = OTHER; note = 2&apos;-O-methoxyethyl
  modified_base order(1^2,26^27): mod_base = OTHER; note = (1,3-dimethylimidazolidin-2-ylidene) phosphoramidate linkage
  modified_base order(2..15,17..26): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 18: mod_base = m5u
gtgctccacctggnacgtatatctctg

SEQ ID NO: 103 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..7,9,11,16,20,22,24..27): mod_base = OTHER; note = 2&apos;-O-methyl
  modified_base order(8,10,12,17,19,21,23): mod_base = OTHER; note = 2&apos;-fluoro
  modified_base order(1^2,26^27): mod_base = OTHER; note = (1,3-dimethylimidazolidin-2-ylidene) phosphoramidate linkage
  modified_base order(4..15,17..26): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 104 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..7,9,11,16,20,22,24..27): mod_base = OTHER; note = 2&apos;-O-methyl
  modified_base order(8,10,12,17,19,21,23): mod_base = OTHER; note = 2&apos;-fluoro
  modified_base order(1^2,26^27): mod_base = OTHER; note = (1,3-dimethylimidazolidin-2-ylidene) phosphoramidate linkage
  modified_base order(4..13,14..16,17..26): mod_base = OTHER; note = phosphorothioate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 105 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..7,9,11,16,20,22,24..27): mod_base = OTHER; note = 2&apos;-O-methyl
  modified_base order(8,10,12,17,19,21,23): mod_base = OTHER; note = 2&apos;-fluoro
  modified_base order(1^2,26^27): mod_base = OTHER; note = (1,3-dimethylimidazolidin-2-ylidene) phosphoramidate linkage
  modified_base order(4..13,14^15,17..26): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 106 - DNA, 27 bp, synthetic construct
  source 1..27: mol_type = other DNA
  misc_feature 15: note = DNA
  misc_feature order(1..14,16..27): note = RNA
  modified_base order(1..7,9,11,16,20,22,24..27): mod_base = OTHER; note = 2&apos;-O-methyl
  modified_base order(8,10,12,17,19,21,23): mod_base = OTHER; note = 2&apos;-fluoro
  modified_base order(13,18): mod_base = OTHER; note = 2&apos;-O-methoxyethyl
  modified_base order(1^2,26^27): mod_base = OTHER; note = (1,3-dimethylimidazolidin-2-ylidene) phosphoramidate linkage
  modified_base order(2..13,14^15,17..26): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 18: mod_base = m5u
gtgctccacctggnacgtatatctctg

SEQ ID NO: 107 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..7,9,11,16,20,22,24..27): mod_base = OTHER; note = 2&apos;-O-methyl
  modified_base order(8,10,12,17,19,21,23): mod_base = OTHER; note = 2&apos;-fluoro
  modified_base order(1^2,21^22,26^27): mod_base = OTHER; note = (1,3-dimethylimidazolidin-2-ylidene) phosphoramidate linkage
  modified_base order(4..13,14^15,17..21,22..26): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 108 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..7,9,11,16,20,22,24..27): mod_base = OTHER; note = 2&apos;-O-methyl
  modified_base order(8,10,12,17,19,21,23): mod_base = OTHER; note = 2&apos;-fluoro
  modified_base order(1^2,21^22,26^27): mod_base = OTHER; note = (1,3-dimethylimidazolidin-2-ylidene) phosphoramidate linkage
  modified_base order(2..13,14^15,17..21,22..26): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 109 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..7,16,24..27): mod_base = OTHER; note = 2&apos;-O-methyl
  modified_base order(8..12,17,19..23): mod_base = OTHER; note = 2&apos;-fluoro
  modified_base order(1^2,21^22,26^27): mod_base = OTHER; note = (1,3-dimethylimidazolidin-2-ylidene) phosphoramidate linkage
  modified_base order(4..13,14^15,17..21,22..26): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 110 - DNA, 27 bp, synthetic construct
  source 1..27: mol_type = other DNA
  misc_feature 15: note = DNA
  misc_feature order(1..14,16..27): note = RNA
  modified_base order(1..3,13,18): mod_base = OTHER; note = 2&apos;-O-methoxyethyl
  modified_base order(4..7,16,24..27): mod_base = OTHER; note = 2&apos;-O-methyl
  modified_base order(8..12,17,19..23): mod_base = OTHER; note = 2&apos;-fluoro
  modified_base order(1^2,21^22,26^27): mod_base = OTHER; note = (1,3-dimethylimidazolidin-2-ylidene) phosphoramidate linkage
  modified_base order(2..13,14^15,17..21,22..26): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctg

SEQ ID NO: 111 - DNA, 28 bp, synthetic construct
  source 1..28: mol_type = other DNA
  misc_feature 16: note = DNA
  misc_feature order(1..15,17..28): note = RNA
  modified_base order(1..4,14,19): mod_base = OTHER; note = 2&apos;-O-methoxyethyl
  modified_base order(5..8,10,12,17,21,23,25..28): mod_base = OTHER; note = 2&apos;-O-methyl
  modified_base order(9,11,13,18,20,22,24): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(5..14,15^16,18..27): mod_base = OTHER; note = phosphorothioate
  modified_base 16^17: mod_base = OTHER; note = methylphosphonate
  modified_base order(3,19): mod_base = m5u
  modified_base 15: mod_base = OTHER; note = 6-amino-5-nitro-3-yl-2(1H)-pyridone nucleobase
ggtgctccacctggnacgtatatctctg

SEQ ID NO: 112 - DNA, 28 bp, synthetic construct
  source 1..28: mol_type = other DNA
  misc_feature 15: note = DNA
  misc_feature order(1..14,16..28): note = RNA
  modified_base order(1..3,13,18): mod_base = OTHER; note = 2&apos;-O-methoxyethyl
  modified_base order(4..7,9,11,16,20,22,24..28): mod_base = OTHER; note = 2&apos;-O-methyl
  modified_base order(8,10,12,17,19,21,23): mod_base = OTHER; note = 2&apos;-fluoro
  modified_base order(1^2,27^28): mod_base = OTHER; note = (1,3-dimethylimidazolidin-2-ylidene) phosphoramidate linkage
  modified_base order(4..13,14^15,17..27): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctctgc

SEQ ID NO: 113 - DNA, 26 bp, synthetic construct
  source 1..26: mol_type = other DNA
  misc_feature 14: note = DNA
  misc_feature order(1..13,15..26): note = RNA
  modified_base order(1..2,12,17): mod_base = OTHER; note = 2&apos;-O-methoxyethyl
  modified_base order(3..6,8,10,15,19,21,23..26): mod_base = OTHER; note = 2&apos;-O-methyl
  modified_base order(7,9,11,16,18,20,22): mod_base = OTHER; note = 2&apos;-fluoro
  modified_base order(1^2,25^26): mod_base = OTHER; note = (1,3-dimethylimidazolidin-2-ylidene) phosphoramidate linkage
  modified_base order(3..12,13^14,16..25): mod_base = OTHER; note = phosphorothioate
  modified_base 14^15: mod_base = OTHER; note = methylphosphonate
  modified_base order(1,17): mod_base = m5u
  modified_base 13: mod_base = OTHER; note = 6-amino-5-nitro-3-yl-2(1H)-pyridone nucleobase
tgctccacctggnacgtatatctctg

SEQ ID NO: 114 - DNA, 26 bp, synthetic construct
  source 1..26: mol_type = other DNA
  misc_feature 15: note = DNA
  misc_feature order(1..14,16..26): note = RNA
  modified_base order(1..3,13,18): mod_base = OTHER; note = 2&apos;-O-methoxyethyl
  modified_base order(4..7,9,11,16,20,22,24..26): mod_base = OTHER; note = 2&apos;-O-methyl
  modified_base order(8,10,12,17,19,21,23): mod_base = OTHER; note = 2&apos;-fluoro
  modified_base order(1^2,25^26): mod_base = OTHER; note = (1,3-dimethylimidazolidin-2-ylidene) phosphoramidate linkage
  modified_base order(4..13,14^15,17..25): mod_base = OTHER; note = phosphorothioate
  modified_base 15^16: mod_base = OTHER; note = methylphosphonate
  modified_base order(2,18): mod_base = m5u
  modified_base 14: mod_base = OTHER; note = 6-amino-5-nitro-3-yl-2(1H)-pyridone nucleobase
gtgctccacctggnacgtatatctct

SEQ ID NO: 115 - DNA, 28 bp, synthetic construct
  source 1..28: mol_type = other DNA
  misc_feature 12: note = DNA
  misc_feature order(1..11,13..28): note = RNA
  modified_base order(1..3,5,7,9,13,16..18,20,22,24): mod_base = OTHER; note = 2&apos;-O-methyl
  modified_base order(4,6,8,14,19,21,23): mod_base = OTHER; note = 2&apos;-fluoro
  modified_base order(10,15,25..28): mod_base = OTHER; note = 2&apos;-O-methoxyethyl
  modified_base order(1..10,11^12,14..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28): mod_base = m5u
  modified_base order(25,27): mod_base = m5c
ctccacctggnacgtatatctctgctct

SEQ ID NO: 116 - DNA, 27 bp, synthetic construct
  source 1..27: mol_type = other DNA
  misc_feature 11: note = DNA
  misc_feature order(1..10,12..27): note = RNA
  modified_base order(1..2,4,6,8,12,15..17,19,21,23): mod_base = OTHER; note = 2&apos;-O-methyl
  modified_base order(3,5,7,13,18,20,22): mod_base = OTHER; note = 2&apos;-fluoro
  modified_base order(9,14,24..27): mod_base = OTHER; note = 2&apos;-O-methoxyethyl
  modified_base order(1..9,10^11,13..24): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base order(14,25,27): mod_base = m5u
  modified_base order(24,26): mod_base = m5c
tccacctggnacgtatatctctgctct

SEQ ID NO: 117 - DNA, 26 bp, synthetic construct
  source 1..26: mol_type = other DNA
  misc_feature 10: note = DNA
  misc_feature order(1..9,11..26): note = RNA
  modified_base order(1,3,5,7,11,14..16,18,20,22): mod_base = OTHER; note = 2&apos;-O-methyl
  modified_base order(2,4,6,12,17,19,21): mod_base = OTHER; note = 2&apos;-fluoro
  modified_base order(8,13,23..26): mod_base = OTHER; note = 2&apos;-O-methoxyethyl
  modified_base order(1..8,9^10,12..23): mod_base = OTHER; note = phosphorothioate
  modified_base 10^11: mod_base = OTHER; note = methylphosphonate
  modified_base 9: mod_base = OTHER; note = 6-amino-5-nitro-3-yl-2(1H)-pyridone nucleobase
  modified_base order(13,24,26): mod_base = m5u
  modified_base order(23,25): mod_base = m5c
ccacctggnacgtatatctctgctct

SEQ ID NO: 118 - DNA, 25 bp, synthetic construct
  source 1..25: mol_type = other DNA
  misc_feature 9: note = DNA
  misc_feature order(1..8,10..25): note = RNA
  modified_base order(1,3,5,11,16,18,20): mod_base = OTHER; note = 2&apos;-fluoro
  modified_base order(2,4,6,10,13..15,17,19,21): mod_base = OTHER; note = 2&apos;-O-methyl
  modified_base order(7,12,22..25): mod_base = OTHER; note = 2&apos;-O-methoxyethyl
  modified_base order(1..7,8^9,11..22): mod_base = OTHER; note = phosphorothioate
  modified_base 9^10: mod_base = OTHER; note = methylphosphonate
  modified_base 8: mod_base = OTHER; note = 6-amino-5-nitro-3-yl-2(1H)-pyridone nucleobase
  modified_base order(12,23,25): mod_base = m5u
  modified_base order(22,24): mod_base = m5c
cacctggnacgtatatctctgctct

SEQ ID NO: 119 - DNA, 24 bp, synthetic construct
  source 1..24: mol_type = other DNA
  misc_feature 8: note = DNA
  misc_feature order(1..7,9..24): note = RNA
  modified_base order(1,3,5,9,12..14,16,18,20): mod_base = OTHER; note = 2&apos;-O-methyl
  modified_base order(2,4,10,15,17,19): mod_base = OTHER; note = 2&apos;-fluoro
  modified_base order(6,11,21..24): mod_base = OTHER; note = 2&apos;-O-methoxyethyl
  modified_base order(1..6,7^8,10..21): mod_base = OTHER; note = phosphorothioate
  modified_base 8^9: mod_base = OTHER; note = methylphosphonate
  modified_base 7: mod_base = OTHER; note = 6-amino-5-nitro-3-yl-2(1H)-pyridone nucleobase
  modified_base order(11,22,24): mod_base = m5u
  modified_base order(21,23): mod_base = m5c
acctggnacgtatatctctgctct

SEQ ID NO: 120 - DNA, 27 bp, synthetic construct
  source 1..27: mol_type = other DNA
  misc_feature 15: note = DNA
  misc_feature order(1..14,16..27): note = RNA
  modified_base order(1..2,7,9,11,17,22,24,26): mod_base = OTHER; note = 2&apos;-fluoro
  modified_base order(3,13,18): mod_base = OTHER; note = 2&apos;-O-methoxyethyl
  modified_base order(4..6,8,10,12,16,19..21,23,25,27): mod_base = OTHER; note = 2&apos;-O-methyl
  modified_base order(1..3,4..13,14^15,17..27): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 18: mod_base = m5u
gtgctccacctggnacgtatatctctg

SEQ ID NO: 121 - DNA, 27 bp, synthetic construct
  source 1..27: mol_type = other DNA
  misc_feature 15: note = DNA
  misc_feature order(1..14,16..27): note = RNA
  modified_base order(1..2,7,9,11,17,22,24,26): mod_base = OTHER; note = 2&apos;-fluoro
  modified_base order(3,13,18): mod_base = OTHER; note = 2&apos;-O-methoxyethyl
  modified_base order(4..6,8,10,12,16,19..21,23,25,27): mod_base = OTHER; note = 2&apos;-O-methyl
  modified_base order(1^2,26^27): mod_base = OTHER; note = (1,3-dimethylimidazolidin-2-ylidene) phosphoramidate linkage
  modified_base order(2^3,4..13,14^15,17..26): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 18: mod_base = m5u
gtgctccacctggnacgtatatctctg

SEQ ID NO: 122 - DNA, 25 bp, synthetic construct
  source 1..25: mol_type = other DNA
  misc_feature 15: note = DNA
  misc_feature order(1..14,16..25): note = RNA
  modified_base order(1..2,7,9,11,17,22,24): mod_base = OTHER; note = 2&apos;-fluoro
  modified_base order(3,13,18): mod_base = OTHER; note = 2&apos;-O-methoxyethyl
  modified_base order(4..6,8,10,12,16,19..21,23,25): mod_base = OTHER; note = 2&apos;-O-methyl
  modified_base order(1..3,4..13,14^15,17..25): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 18: mod_base = m5u
gtgctccacctggnacgtatatctc

SEQ ID NO: 123 - DNA, 25 bp, synthetic construct
  source 1..25: mol_type = other DNA
  misc_feature 15: note = DNA
  misc_feature order(1..14,16..25): note = RNA
  modified_base order(1..2,7,9,11,17,22,24): mod_base = OTHER; note = 2&apos;-fluoro
  modified_base order(3,13,18): mod_base = OTHER; note = 2&apos;-O-methoxyethyl
  modified_base order(4..6,8,10,12,16,19..21,23,25): mod_base = OTHER; note = 2&apos;-O-methyl
  modified_base order(1^2,24^25): mod_base = OTHER; note = (1,3-dimethylimidazolidin-2-ylidene) phosphoramidate linkage
  modified_base order(2^3,4..13,14^15,17..24): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 18: mod_base = m5u
gtgctccacctggnacgtatatctc

SEQ ID NO: 124 - DNA, 24 bp, synthetic construct
  source 1..24: mol_type = other DNA
  misc_feature 15: note = DNA
  misc_feature order(1..14,16..24): note = RNA
  modified_base order(1..2,7,9,11,17,22,24): mod_base = OTHER; note = 2&apos;-fluoro
  modified_base order(3,13,18): mod_base = OTHER; note = 2&apos;-O-methoxyethyl
  modified_base order(4..6,8,10,12,16,19..21,23): mod_base = OTHER; note = 2&apos;-O-methyl
  modified_base order(1..3,4..13,14^15,17..24): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 18: mod_base = m5u
gtgctccacctggnacgtatatct

SEQ ID NO: 125 - DNA, 24 bp, synthetic construct
  source 1..24: mol_type = other DNA
  misc_feature 15: note = DNA
  misc_feature order(1..14,16..24): note = RNA
  modified_base order(1..2,7,9,11,17,22,24): mod_base = OTHER; note = 2&apos;-fluoro
  modified_base order(3,13,18): mod_base = OTHER; note = 2&apos;-O-methoxyethyl
  modified_base order(4..6,8,10,12,16,19..21,23): mod_base = OTHER; note = 2&apos;-O-methyl
  modified_base order(1^2,23^24): mod_base = OTHER; note = (1,3-dimethylimidazolidin-2-ylidene) phosphoramidate linkage
  modified_base order(2^3,4..13,14^15,17..23): mod_base = OTHER; note = phosphorothioate
  modified_base 15^16: mod_base = OTHER; note = methylphosphonate
  modified_base 14: mod_base = OTHER; note = 6-amino-5-nitro-3-yl-2(1H)-pyridone nucleobase
  modified_base 18: mod_base = m5u
gtgctccacctggnacgtatatct

SEQ ID NO: 126 - DNA, 23 bp, synthetic construct
  source 1..23: mol_type = other DNA
  misc_feature 11: note = DNA
  misc_feature order(1..10,12..23): note = RNA
  modified_base order(1..2,4,6,8,12,15,17,19,21..23): mod_base = OTHER; note = 2&apos;-O-methyl
  modified_base order(3,5,7,13,16,18,20): mod_base = OTHER; note = 2&apos;-fluoro
  modified_base order(9,14): mod_base = OTHER; note = 2&apos;-O-methoxyethyl
  modified_base order(1..9,10^11,13..23): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 127 - DNA, 23 bp, synthetic construct
  source 1..23: mol_type = other DNA
  misc_feature 11: note = DNA
  misc_feature order(1..10,12..23): note = RNA
  modified_base order(1..2,4,6,8,12,15,17,19,21,23): mod_base = OTHER; note = 2&apos;-O-methyl
  modified_base order(3,5,7,13,16,18,20,22): mod_base = OTHER; note = 2&apos;-fluoro
  modified_base order(9,14): mod_base = OTHER; note = 2&apos;-O-methoxyethyl
  modified_base order(1..9,10^11,13..23): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 128 - DNA, 23 bp, synthetic construct
  source 1..23: mol_type = other DNA
  misc_feature 11: note = DNA
  misc_feature order(1..10,12..23): note = RNA
  modified_base order(1..2,4,6,8,12,15..17,19,21,23): mod_base = OTHER; note = 2&apos;-O-methyl
  modified_base order(3,5,7,13,18,20,22): mod_base = OTHER; note = 2&apos;-fluoro
  modified_base order(9,14): mod_base = OTHER; note = 2&apos;-O-methoxyethyl
  modified_base order(1..9,10^11,13..23): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 129 - DNA, 23 bp, synthetic construct
  source 1..23: mol_type = other DNA
  misc_feature 11: note = DNA
  misc_feature order(1..10,12..23): note = RNA
  modified_base order(1..2,6,8,12,15..17,19,21,23): mod_base = OTHER; note = 2&apos;-O-methyl
  modified_base order(3..5,9,14): mod_base = OTHER; note = 2&apos;-O-methoxyethyl
  modified_base order(7,13,18,20,22): mod_base = OTHER; note = 2&apos;-fluoro
  modified_base order(1..3,5..9,10^11,13..23): mod_base = OTHER; note = phosphorothioate
  modified_base 11^12: mod_base = OTHER; note = methylphosphonate
  modified_base order(3,5): mod_base = m5c
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 130 - DNA, 23 bp, synthetic construct
  source 1..23: mol_type = other DNA
  misc_feature 11: note = DNA
  misc_feature order(1..10,12..23): note = RNA
  modified_base order(1..2,6,8,12,15..17,19,21,23): mod_base = OTHER; note = 2&apos;-O-methyl
  modified_base order(3..4,9,14): mod_base = OTHER; note = 2&apos;-O-methoxyethyl
  modified_base order(5,7,13,18,20,22): mod_base = OTHER; note = 2&apos;-fluoro
  modified_base order(1..3,4..9,10^11,13..23): mod_base = OTHER; note = phosphorothioate
  modified_base 11^12: mod_base = OTHER; note = methylphosphonate
  modified_base 3: mod_base = m5c
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 131 - DNA, 23 bp, synthetic construct
  source 1..23: mol_type = other DNA
  misc_feature 11: note = DNA
  misc_feature order(1..10,12..23): note = RNA
  modified_base order(1..2,4,6,8,12,15..17,19,21,23): mod_base = OTHER; note = 2&apos;-O-methyl
  modified_base order(3,9,14): mod_base = OTHER; note = 2&apos;-O-methoxyethyl
  modified_base order(5,7,13,18,20,22): mod_base = OTHER; note = 2&apos;-fluoro
  modified_base order(1..9,10^11,13..23): mod_base = OTHER; note = phosphorothioate
  modified_base 11^12: mod_base = OTHER; note = methylphosphonate
  modified_base 3: mod_base = m5c
  modified_base 10: mod_base = OTHER; note = 6-amino-5-nitro-3-yl-2(1H)-pyridone nucleobase
  modified_base 14: mod_base = m5u
tccccctggnacgtatatctctg

SEQ ID NO: 132 - DNA, 23 bp, synthetic construct
  source 1..23: mol_type = other DNA
  misc_feature 11: note = DNA
  misc_feature order(1..10,12..23): note = RNA
  modified_base order(1..2,6,8,12,15..17,19,21,23): mod_base = OTHER; note = 2&apos;-O-methyl
  modified_base order(3..5,9,14): mod_base = OTHER; note = 2&apos;-O-methoxyethyl
  modified_base order(7,13,18,20,22): mod_base = OTHER; note = 2&apos;-fluoro
  modified_base order(1..3,6..9,10^11,13..23): mod_base = OTHER; note = phosphorothioate
  modified_base 11^12: mod_base = OTHER; note = methylphosphonate
  modified_base order(3,5): mod_base = m5c
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 133 - DNA, 23 bp, synthetic construct
  source 1..23: mol_type = other DNA
  misc_feature 11: note = DNA
  misc_feature order(1..10,12..23): note = RNA
  modified_base order(1..2,6,8,12,15..17,19,21,23): mod_base = OTHER; note = 2&apos;-O-methyl
  modified_base order(3..4,9,14): mod_base = OTHER; note = 2&apos;-O-methoxyethyl
  modified_base order(5,7,13,18,20,22): mod_base = OTHER; note = 2&apos;-fluoro
  modified_base order(1..3,5..9,10^11,13..23): mod_base = OTHER; note = phosphorothioate
  modified_base 11^12: mod_base = OTHER; note = methylphosphonate
  modified_base 3: mod_base = m5c
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 134 - DNA, 23 bp, synthetic construct
  source 1..23: mol_type = other DNA
  misc_feature 11: note = DNA
  misc_feature order(1..10,12..23): note = RNA
  modified_base order(1..2,4,6,8,12,15..17,19,21,23): mod_base = OTHER; note = 2&apos;-O-methyl
  modified_base order(3,9,14): mod_base = OTHER; note = 2&apos;-O-methoxyethyl
  modified_base order(5,7,13,18,20,22): mod_base = OTHER; note = 2&apos;-fluoro
  modified_base order(1..3,4..9,10^11,13..23): mod_base = OTHER; note = phosphorothioate
  modified_base 11^12: mod_base = OTHER; note = methylphosphonate
  modified_base 3: mod_base = m5c
  modified_base 10: mod_base = OTHER; note = 6-amino-5-nitro-3-yl-2(1H)-pyridone nucleobase
  modified_base 14: mod_base = m5u
tccccctggnacgtatatctctg

SEQ ID NO: 135 - DNA, 23 bp, synthetic construct
  source 1..23: mol_type = other DNA
  misc_feature 11: note = DNA
  misc_feature order(1..10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1^2,22^23): mod_base = OTHER; note = (1,3-dimethylimidazolidin-2-ylidene) phosphoramidate linkage
  modified_base order(2..9,10^11,13..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 136 - DNA, 23 bp, synthetic construct
  source 1..23: mol_type = other DNA
  misc_feature 11: note = DNA
  misc_feature order(1..10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 137 - DNA, 23 bp, synthetic construct
  source 1..23: mol_type = other DNA
  misc_feature 11: note = DNA
  misc_feature order(1..10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1^2,6^7,17^18,22^23): mod_base = OTHER; note = (1,3-dimethylimidazolidin-2-ylidene) phosphoramidate linkage
  modified_base order(2..6,7..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 138 - DNA, 23 bp, synthetic construct
  source 1..23: mol_type = other DNA
  misc_feature 11: note = DNA
  misc_feature order(1..10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1^2,11^12,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 139 - DNA, 23 bp, synthetic construct
  source 1..23: mol_type = other DNA
  misc_feature 11: note = DNA
  misc_feature order(1..10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctttg

SEQ ID NO: 140 - DNA, 23 bp, synthetic construct
  source 1..23: mol_type = other DNA
  misc_feature 11: note = DNA
  misc_feature order(1..10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatttctg

SEQ ID NO: 141 - DNA, 23 bp, synthetic construct
  source 1..23: mol_type = other DNA
  misc_feature 11: note = DNA
  misc_feature order(1..10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatttttg

SEQ ID NO: 142 - DNA, 23 bp, synthetic construct
  source 1..23: mol_type = other DNA
  misc_feature order(9,11): note = DNA
  misc_feature order(1..8,10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base 14: mod_base = OTHER; note = 2&apos;-O-methoxyethyl
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 143 - DNA, 23 bp, synthetic construct
  source 1..23: mol_type = other DNA
  misc_feature order(9,11): note = DNA
  misc_feature order(1..8,10,12..23): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base 14: mod_base = OTHER; note = 2&apos;-O-methoxyethyl
  modified_base order(1^2,11^12,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 144 - DNA, 23 bp, synthetic construct
  source 1..23: mol_type = other DNA
  misc_feature 11: note = DNA
  misc_feature order(1..10,12..23): note = RNA
  modified_base order(1..8,12,15..23): mod_base = OTHER; note = 2&apos;-O-methyl
  modified_base order(9,14): mod_base = OTHER; note = 2&apos;-O-methoxyethyl
  modified_base 13: mod_base = OTHER; note = 2&apos;-fluoro
  modified_base order(1..9,10^11,13..23): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 145 - DNA, 23 bp, synthetic construct
  source 1..23: mol_type = other DNA
  misc_feature 11: note = DNA
  misc_feature order(1..10,12..23): note = RNA
  modified_base order(1..8,12,15..23): mod_base = OTHER; note = 2&apos;-O-methyl
  modified_base order(9,14): mod_base = OTHER; note = 2&apos;-O-methoxyethyl
  modified_base 13: mod_base = OTHER; note = 2&apos;-fluoro
  modified_base order(1^2,22^23): mod_base = OTHER; note = (1,3-dimethylimidazolidin-2-ylidene) phosphoramidate linkage
  modified_base order(2..9,10^11,13..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 146 - DNA, 23 bp, synthetic construct
  source 1..23: mol_type = other DNA
  misc_feature 11: note = DNA
  misc_feature order(1..10,12..23): note = RNA
  modified_base order(1..8,12,15..23): mod_base = OTHER; note = 2&apos;-O-methyl
  modified_base order(9,14): mod_base = OTHER; note = 2&apos;-O-methoxyethyl
  modified_base 13: mod_base = OTHER; note = 2&apos;-fluoro
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 147 - DNA, 23 bp, synthetic construct
  source 1..23: mol_type = other DNA
  misc_feature 11: note = DNA
  misc_feature order(1..10,12..23): note = RNA
  modified_base order(1..3,12,20..23): mod_base = OTHER; note = 2&apos;-O-methyl
  modified_base order(4..8,13,15..19): mod_base = OTHER; note = 2&apos;-fluoro
  modified_base order(9,14): mod_base = OTHER; note = 2&apos;-O-methoxyethyl
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 148 - DNA, 23 bp, synthetic construct
  source 1..23: mol_type = other DNA
  misc_feature 11: note = DNA
  misc_feature order(1..10,12..23): note = RNA
  modified_base order(1..3,12,16,18,20..23): mod_base = OTHER; note = 2&apos;-O-methyl
  modified_base order(4..8,13,15,17,19): mod_base = OTHER; note = 2&apos;-fluoro
  modified_base order(9,14): mod_base = OTHER; note = 2&apos;-O-methoxyethyl
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 149 - DNA, 23 bp, synthetic construct
  source 1..23: mol_type = other DNA
  misc_feature 11: note = DNA
  misc_feature order(1..10,12..23): note = RNA
  modified_base order(1..3,7,12,16,18,20..23): mod_base = OTHER; note = 2&apos;-O-methyl
  modified_base order(4..6,8,13,15,17,19): mod_base = OTHER; note = 2&apos;-fluoro
  modified_base order(9,14): mod_base = OTHER; note = 2&apos;-O-methoxyethyl
  modified_base order(1^2,17^18,22^23): mod_base = OTHER; note = (1,3-dimethylimidazolidin-2-ylidene) phosphoramidate linkage
  modified_base order(2..9,10^11,13..17,18..22): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctctg

SEQ ID NO: 150 - DNA, 23 bp, synthetic construct
  source 1..23: mol_type = other DNA
  misc_feature 12: note = DNA
  misc_feature order(1..11,13..23): note = RNA
  modified_base order(1..4,6,8,13,17,19,21..23): mod_base = OTHER; note = 2&apos;-O-methyl
  modified_base order(5,7,9,14,16,18,20): mod_base = OTHER; note = 2&apos;-fluoro
  modified_base order(10,15): mod_base = OTHER; note = 2&apos;-O-methoxyethyl
  modified_base order(1..10,11^12,14..23): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base 15: mod_base = m5u
ctccacctggnacgtatatctct

SEQ ID NO: 151 - DNA, 23 bp, synthetic construct
  source 1..23: mol_type = other DNA
  misc_feature 12: note = DNA
  misc_feature order(1..11,13..23): note = RNA
  modified_base order(1..4,6,8,13,17,19,21..23): mod_base = OTHER; note = 2&apos;-O-methyl
  modified_base order(5,7,9,14,16,18,20): mod_base = OTHER; note = 2&apos;-fluoro
  modified_base order(10,15): mod_base = OTHER; note = 2&apos;-O-methoxyethyl
  modified_base order(1^2,18^19,22^23): mod_base = OTHER; note = (1,3-dimethylimidazolidin-2-ylidene) phosphoramidate linkage
  modified_base order(2..10,11^12,14..18,19..22): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base 15: mod_base = m5u
ctccacctggnacgtatatctct

SEQ ID NO: 152 - DNA, 24 bp, synthetic construct
  source 1..24: mol_type = other DNA
  misc_feature 12: note = DNA
  misc_feature order(1..11,13..24): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15): mod_base = OTHER; note = 2&apos;-O-methoxyethyl
  modified_base order(1^2,18^19,23^24): mod_base = OTHER; note = (1,3-dimethylimidazolidin-2-ylidene) phosphoramidate linkage
  modified_base order(2..10,11^12,14..18,19..23): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base 15: mod_base = m5u
ctccacctggnacgtatatctctg

SEQ ID NO: 153 - DNA, 22 bp, synthetic construct
  source 1..22: mol_type = other DNA
  misc_feature 11: note = DNA
  misc_feature order(1..10,12..22): note = RNA
  modified_base order(1..3,5,7,12,16,18,20..22): mod_base = OTHER; note = 2&apos;-O-methyl
  modified_base order(4,6,8,13,15,17,19): mod_base = OTHER; note = 2&apos;-fluoro
  modified_base order(9,14): mod_base = OTHER; note = 2&apos;-O-methoxyethyl
  modified_base order(1^2,17^18,21^22): mod_base = OTHER; note = (1,3-dimethylimidazolidin-2-ylidene) phosphoramidate linkage
  modified_base order(2..9,10^11,13..17,18..21): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base 14: mod_base = m5u
tccacctggnacgtatatctct

SEQ ID NO: 154 - DNA, 28 bp, synthetic construct
  source 1..28: mol_type = other DNA
  misc_feature 11: note = DNA
  misc_feature order(1..10,12..28): note = RNA
  modified_base order(1..3,5,7,12,16,18,20..23): mod_base = OTHER; note = 2&apos;-O-methyl
  modified_base order(4,6,8,13,15,17,19): mod_base = OTHER; note = 2&apos;-fluoro
  modified_base order(9,14,24..28): mod_base = OTHER; note = 2&apos;-O-methoxyethyl
  modified_base order(1^2,17^18,27^28): mod_base = OTHER; note = (1,3-dimethylimidazolidin-2-ylidene) phosphoramidate linkage
  modified_base order(2..9,10^11,13..17,18..24): mod_base = OTHER; note = phosphorothioate
  modified_base 11^12: mod_base = OTHER; note = methylphosphonate
  modified_base 10: mod_base = OTHER; note = 6-amino-5-nitro-3-yl-2(1H)-pyridone nucleobase
  modified_base order(14,25,27..28): mod_base = m5u
  modified_base order(24,26): mod_base = m5c
tccacctggnacgtatatctctgctctt

SEQ ID NO: 155 - DNA, 30 bp, synthetic construct; identical to SEQ ID NO: 76
  source 1..30: mol_type = other DNA
  misc_feature 12: note = DNA
  misc_feature order(1..11,13..30): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..30): mod_base = OTHER; note = 2&apos;-O-methoxyethyl
  modified_base order(1^2,18^19,29^30): mod_base = OTHER; note = (1,3-dimethylimidazolidin-2-ylidene) phosphoramidate linkage
  modified_base order(2..10,11^12,14..18,19..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30): mod_base = m5c

SEQ ID NO: 156 - DNA, 30 bp, synthetic construct; identical to SEQ ID NO: 76
  source 1..30: mol_type = other DNA
  misc_feature 12: note = DNA
  misc_feature order(1..11,13..30): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..30): mod_base = OTHER; note = 2&apos;-O-methoxyethyl
  modified_base order(1^2,18^19,24^25,29^30): mod_base = OTHER; note = (1,3-dimethylimidazolidin-2-ylidene) phosphoramidate linkage
  modified_base order(2..10,11^12,14..18,19..24): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30): mod_base = m5c

SEQ ID NO: 157 - DNA, 30 bp, synthetic construct; identical to SEQ ID NO: 76
  source 1..30: mol_type = other DNA
  misc_feature 12: note = DNA
  misc_feature order(1..11,13..30): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..30): mod_base = OTHER; note = 2&apos;-O-methoxyethyl
  modified_base order(1^2,18^19,29^30): mod_base = OTHER; note = (1,3-dimethylimidazolidin-2-ylidene) phosphoramidate linkage
  modified_base order(2..10,11^12,14..18,19..29): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30): mod_base = m5c

SEQ ID NO: 158 - DNA, 30 bp, synthetic construct; identical to SEQ ID NO: 76
  source 1..30: mol_type = other DNA
  misc_feature 12: note = DNA
  misc_feature order(1..11,13..30): note = RNA
  modified_base order(1..2,4,6,8,13,17,19,21..24): mod_base = OTHER; note = 2&apos;-O-methyl
  modified_base order(3,5,7,9,14,16,18,20): mod_base = OTHER; note = 2&apos;-fluoro
  modified_base order(10,15,25..30): mod_base = OTHER; note = 2&apos;-O-methoxyethyl
  modified_base order(1^2,18^19,29^30): mod_base = OTHER; note = (1,3-dimethylimidazolidin-2-ylidene) phosphoramidate linkage
  modified_base order(2..10,11^12,14..18,19..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30): mod_base = m5c

SEQ ID NO: 159 - DNA, 30 bp, synthetic construct
  source 1..30: mol_type = other DNA
  misc_feature 12: note = DNA
  misc_feature order(1..11,13..30): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..30): mod_base = OTHER; note = 2&apos;-O-methoxyethyl
  modified_base order(1^2,18^19,29^30): mod_base = OTHER; note = (1,3-dimethylimidazolidin-2-ylidene) phosphoramidate linkage
  modified_base order(2..10,11^12,14..18,19..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30): mod_base = m5c
ctctacctggnacgtatatctctgctcttc

SEQ ID NO: 160 - DNA, 30 bp, synthetic construct
  source 1..30: mol_type = other DNA
  misc_feature 12: note = DNA
  misc_feature order(1..11,13..30): note = RNA
  modified_base order(1..4,6,8,13,17,19,21..24): mod_base = OTHER; note = 2&apos;-O-methyl
  modified_base order(5,7,9,14,16,18,20): mod_base = OTHER; note = 2&apos;-fluoro
  modified_base order(10,15,25..30): mod_base = OTHER; note = 2&apos;-O-methoxyethyl
  modified_base order(1^2,18^19,29^30): mod_base = OTHER; note = (1,3-dimethylimidazolidin-2-ylidene) phosphoramidate linkage
  modified_base order(2..10,11^12,14..18,19..25): mod_base = OTHER; note = phosphorothioate
  modified_base 12^13: mod_base = OTHER; note = methylphosphonate
  modified_base 11: mod_base = OTHER; note = 6-amino-5-nitro-3-yl-2(1H)-pyridone nucleobase
  modified_base order(15,26,28..29): mod_base = m5u
  modified_base order(25,27,30): mod_base = m5c
ctctatctggnacgtatatctctgctcttc

SEQ ID NO: 161 - DNA, 30 bp, synthetic construct; identical to SEQ ID NO: 85
  source 1..30: mol_type = other DNA
  misc_feature 25: note = DNA
  misc_feature order(1..24,26..30): note = RNA
  modified_base order(1..3,10,12,18..20,26,29..30): mod_base = OTHER; note = 2&apos;-O-methyl
  modified_base order(4..5,8..9,11,13..15,17,22,27): mod_base = OTHER; note = 2&apos;-fluoro
  modified_base order(6..7,16,21,23,28): mod_base = OTHER; note = 2&apos;-O-methoxyethyl
  modified_base order(1^2,11^12,14^15,19^20,29^30): mod_base = OTHER; note = (1,3-dimethylimidazolidin-2-ylidene) phosphoramidate linkage
  modified_base order(2..6,8^9,10^11,13^14,16..19,22..25,27..29): mod_base = OTHER; note = phosphorothioate
  modified_base 25^26: mod_base = OTHER; note = methylphosphonate
  modified_base order(6..7,16): mod_base = m5c
  modified_base order(21,28): mod_base = m5u
  modified_base 24: mod_base = OTHER; note = 6-amino-5-nitro-3-yl-2(1H)-pyridone nucleobase

SEQ ID NO: 162 - DNA, 30 bp, synthetic construct; identical to SEQ ID NO: 85
  source 1..30: mol_type = other DNA
  misc_feature 25: note = DNA
  misc_feature order(1..24,26..30): note = RNA
  modified_base order(1..3,12,26,29..30): mod_base = OTHER; note = 2&apos;-O-methyl
  modified_base order(4..5,8..9,11,13..15,17..19,22,27): mod_base = OTHER; note = 2&apos;-fluoro
  modified_base order(6..7,10,16,20..21,23,28): mod_base = OTHER; note = 2&apos;-O-methoxyethyl
  modified_base order(1^2,11^12,14^15,19^20,29^30): mod_base = OTHER; note = (1,3-dimethylimidazolidin-2-ylidene) phosphoramidate linkage
  modified_base order(2..6,8^9,10^11,13^14,16..19,22..25,27..29): mod_base = OTHER; note = phosphorothioate
  modified_base 25^26: mod_base = OTHER; note = methylphosphonate
  modified_base order(6..7,16,20): mod_base = m5c
  modified_base order(21,28): mod_base = m5u
  modified_base 24: mod_base = OTHER; note = 6-amino-5-nitro-3-yl-2(1H)-pyridone nucleobase

SEQ ID NO: 163 - DNA, 30 bp, synthetic construct; identical to SEQ ID NO: 85
  source 1..30: mol_type = other DNA
  misc_feature order(23,25): note = DNA
  misc_feature order(1..22,24,26..30): note = RNA
  modified_base order(1..3,6..7,10,12,16,20..21,26,28..30): mod_base = OTHER; note = 2&apos;-O-methyl
  modified_base order(4..5,8..9,11,13..15,17..19,22,27): mod_base = OTHER; note = 2&apos;-fluoro
  modified_base order(1^2,11^12,14^15,29^30): mod_base = OTHER; note = (1,3-dimethylimidazolidin-2-ylidene) phosphoramidate linkage
  modified_base order(2..6,7..9,10^11,13^14,16..20,22..25,27..29): mod_base = OTHER; note = phosphorothioate
  modified_base 25^26: mod_base = OTHER; note = methylphosphonate
  modified_base 24: mod_base = OTHER; note = 6-amino-5-nitro-3-yl-2(1H)-pyridone nucleobase

SEQ ID NO: 164 - DNA, 30 bp, synthetic construct; identical to SEQ ID NO: 85
  source 1..30: mol_type = other DNA
  misc_feature order(23,25): note = DNA
  misc_feature order(1..22,24,26..30): note = RNA
  modified_base order(1..3,9,11..12,15,17,26,28..30): mod_base = OTHER; note = 2&apos;-O-methyl
  modified_base order(4..5,8,13..14,18..19,22,27): mod_base = OTHER; note = 2&apos;-fluoro
  modified_base order(6..7,10,16,20..21): mod_base = OTHER; note = 2&apos;-O-methoxyethyl
  modified_base order(1^2,11^12,14^15,19^20,29^30): mod_base = OTHER; note = (1,3-dimethylimidazolidin-2-ylidene) phosphoramidate linkage
  modified_base order(2..6,8^9,10^11,13^14,17..19,22..25,27..29): mod_base = OTHER; note = phosphorothioate
  modified_base 25^26: mod_base = OTHER; note = methylphosphonate
  modified_base order(6..7,16,20): mod_base = m5c
  modified_base 21: mod_base = m5u
  modified_base 24: mod_base = OTHER; note = 6-amino-5-nitro-3-yl-2(1H)-pyridone nucleobase

(19) United States
(12) Patent Application Publication
(10) Pub. No.: US 2026/0263636 A1
(43) Pub. Date: Sep. 10, 2026

(54) ANTISENSE OLIGONUCLEOTIDES FOR THE TREATMENT OF HEREDITARY HFE-HEMOCHROMATOSIS

(71) Applicant: ProQR Therapeutics, Inc., Leiden (NL)

(72) Inventors: Marko POTMAN, Leiden (NL); Aliye Seda YILMAZ-ELIS, Leiden (NL); Aron KOS, Leiden (NL)

(73) Assignee: ProQR Therapeutics, Inc., Leiden (NL)

(21) Appl. No.: 19/132,765

(22) PCT Filed: Nov. 23, 2023
(86) PCT No.: PCT/EP2023/082797

§ 371 Date: May 23, 2025

(30) Foreign Application Priority Data
Nov. 24, 2022 (GB) 2217580.6
Apr. 25, 2023 (GB) 2306060.1

(51) Int. Cl. A61K 48/00; A61K 38/17; A61P 3/00; C12N 15/11; C12N 15/78; C12N 15/86; C12N 15/88

(52) U.S. Cl. CPC A61K 48/005; A61K 38/1709; A61P 3/00; C12N 15/111; C12N 15/78; C12N 15/86; C12N 15/88; C12N 2310/20; C12Y 305/04004

(57) ABSTRACT

The disclosure relates to the field of diseases caused by iron overload, such as homeostatic iron regulator protein (HFE) hemochromatosis. The disclosure provides oligonucleotides for RNA editing technology in targeting and deaminating the c.845G>A nucleotide in transcripts of the p.Cys282Tyr (C282Y) mutant human HFE gene to reduce iron overload, especially in the liver.

### TECHNICAL FIELD

This invention relates to the field of medicine. It relates to the field of diseases caused by iron overload, such as homeostatic iron regulator protein (HFE)-related hemochromatosis. The invention involves the use of nucleotide editing technology in targeting the HFE gene and transcript to bring about amino acid changes that restore the normal function of the homeostatic iron regulator (HFE) protein in regulating body iron homeostasis.

### BACKGROUND

Iron overload disorders represent an important class of human diseases. Of the primary iron overload conditions, by far the most common and best studied is HFE-related hemochromatosis (HH). The most prevalent HH-causing mutation in humans is the C282Y substitution in HFE resulting in a disruption of the iron homeostasis (Milman N T et al. 2019. Gastroenterology Res. 12(5):221-232; Anderson G J & Bardou-Jacquet E. 2021. Ann Transl Med. 9(80):731; Barton J C & Edwards C Q. 2018. GeneReviews. Seattle (WA) University of Washington. 1993, updated Dec. 6, 2018; Brissot P et al. 2018. Nat Rev Dis Primers. 4:18016; Cancado R D et al. 2022. Hematol Transfus Cell Ther. 44(1):95-99; Ye Q et al. 2016. PloS One. 11(9):e0163423). This disease is characterized by reduced expression of the iron-regulatory hormone hepcidin, leading to increased dietary iron absorption and iron deposition in multiple tissues including liver, pancreas, joints, heart, and pituitary. The phenotype of HH is quite variable, with some individuals showing little or no evidence of increased body iron, yet others showing severe iron loading, tissue damage and clinical sequelae. Most genetically predisposed individuals show at least some evidence of iron loading (increased transferrin saturation and serum ferritin). Because it is such a common condition in Caucasian populations (1:200 to 1:500), and even though a minority of the affected individuals show clinical symptoms, it remains an important clinical entity. Early symptoms that do occur include abdominal pain, weakness, lethargy, weight loss, arthralgias, erectile dysfunction in men, diminished libido in women due to hypogonadism, loss of muscle mass, osteoporosis, diabetes mellitus, and increased risk of cirrhosis when the serum ferritin is higher than 1000 ng/mL. Other findings may include progressive increase in skin pigmentation, congestive heart failure, and/or arrhythmias, arthritis, and hypogonadism. Individuals with HH have inappropriately high absorption of iron from a normal diet by the mucosa of the small intestine, resulting in excessive parenchymal storage of iron, which may result in damage to target organs and, potentially, organ failure.

Men are far more likely to manifest significant disease than women, with the latter losing iron predominantly through menstrual blood loss. Other forms of blood loss, immune system influences, the amount of bioavailable iron in the diet and lifestyle factors such as high alcohol intake can also contribute to iron loading and disease expression. Cirrhosis is more common among C282Y homozygotes who consume more than 60 g of alcohol per day. Symptoms related to iron overload usually appear between age 40 and 60 years in men and after menopause in women. Occasionally, HH manifests at an earlier age, but hepatic fibrosis or cirrhosis is rare before age 40 years. In general, it is held that the development of cirrhosis determines whether an individual has a normal life expectancy or a decreased life expectancy even with iron depletion therapy, primarily due to the development of hepatocellular cancer. Treatment of patients with cirrhosis to achieve iron depletion does not eliminate the 10%-30% risk of primary liver cancer. In general, death in individuals with clinical HH is often caused by liver failure, primary liver cancer, extrahepatic cancers, congestive heart failure, or arrhythmia. Earlier screening studies showed that 38% to 50% of C282Y homozygotes develop iron overload and 10% to 33% eventually develop hemochromatosis-related symptoms or organ damage. Some individuals who are heterozygous for C282Y have elevated serum transferrin saturation and serum ferritin concentrations, but they typically do not develop complications of iron overload, although this may occur due to environmental effects, lifestyle, or other mutations (such as the H63D in HFE or in another iron homeostasis gene). Individuals that are homozygous for the C282Y mutation can be asymptomatic for decades and subsequently show the manifestation of symptoms at approximately 40 years of age in men and approximately 50 years of age in women. Patients with the C282Y mutation have a worse quality of life, measured by the short form healthy survey (SF-36) scale, as compared to patients with other genotypes (Fonseca et al. 2018. BMC Med Genet. 19(1):3).

Phlebotomy (venesection therapy) is the standard treatment for patients with hemochromatosis. It is very effective to prevent hemochromatosis damage, is safe, and has a low cost. Early diagnosis and initiation of phlebotomies are important actions to prevent tissue and cell damages due to reactive oxygen species from the iron overload. Nevertheless, phlebotomy is not always sufficient, and elderly people often do not tolerate the regimen. Iron chelation therapy is not indicated for classical hemochromatosis, although in rare cases iron chelators are an adjuvant treatment, or alternative, such as in severe iron overload without efficacy with phlebotomies and/or poor vein conditions. Erythrocytapheresis have been used to treat hemochromatosis patients but is more expensive and less available than phlebotomy. Two studies showed the importance of the adequate hemochromatosis treatment of the early intervention to prevent morbidity caused by the hemochromatosis related to the HFE C282Y homozygosity. Ong et al. (Lancet Haematol. 2017; 4(12):e607-614), enrolling patients with the C282Y homozygous genotype and with a moderate serum ferritin level (300-1000 μg/L), performed a randomized controlled trial by dividing the cohort into two groups: iron reduction by erythrocytapheresis (treatment) or sham treatment by plasmapheresis (control). They identified an improvement in the modified fatigue impact scale (MFIS) score in the treatment group, compared to the control. Pilling et al. (BMJ. 2019;364:k5222), in a large cohort study at the UK biobank including 2,890 patients with the C282Y homozygous genotype, concluded that hemochromatosis was associated with significant prevalent and incident clinically diagnosed morbidity (liver disease, rheumatoid arthritis, osteoarthritis and diabetes) in both males and females (Cancado et al. 2022, supra).

The present disclosure aims to provide one or more alternative, and/or improved, compounds or compositions for use in the treatment of hereditary HH.

### SUMMARY OF THE INVENTION

Disclosed herein is an RNA editing oligonucleotide (EON) capable of forming a double-stranded (ds) complex with a region of an endogenous human HFE transcript molecule in a cell, wherein the region of the HFE transcript molecule comprises a target adenosine, and wherein the ds complex can recruit an endogenous ADAR enzyme to deaminate the target adenosine into an inosine, thereby editing the HFE transcript molecule. Preferably, the HFE transcript molecule is a pre-mRNA or an mRNA molecule. Preferably, the cell is a human liver cell, more preferably a hepatocyte. In a preferred aspect, the target adenosine is a c.845G>A mutation in the HFE gene. When the EON is in a naked form, it is preferred that at least one nucleotide comprises one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution.

Disclosed herein is also a vector, preferably a viral vector, more preferably an adeno-associated virus (AAV) vector, comprising a nucleic acid molecule encoding an EON capable of forming a ds complex with a region of an endogenous human HFE transcript molecule in a cell, wherein the region of the HFE transcript molecule comprises a target adenosine, and wherein the ds complex can recruit an endogenous ADAR enzyme to deaminate the target adenosine into an inosine.

Disclosed herein is also a pharmaceutical composition comprising an EON as disclosed, or a vector as disclosed, and a pharmaceutically acceptable carrier.

Disclosed herein is also an EON capable of forming a ds complex with a region of an endogenous human HFE transcript molecule in a cell, wherein the region of the HFE transcript molecule comprises a target adenosine, and wherein the ds complex can recruit an endogenous ADAR enzyme to deaminate the target adenosine into an inosine for use in the treatment of HFE hemochromatosis.

Disclosed is a method of editing a HFE polynucleotide, the method comprising contacting the HFE polynucleotide with an EON capable of effecting an adenosine deaminase acting on RNA (ADAR)-mediated adenosine to inosine alteration of an adenosine associated with iron homeostasis, thereby editing the HFE polynucleotide. Disclosed is a method of treating HFE hemochromatosis in a patient in need thereof, the method comprising contacting a HFE polynucleotide in a cell of the subject with an EON capable of effecting an ADAR-mediated adenosine to inosine alteration of an adenosine associated with iron homeostasis, thereby treating the patient.

### BRIEF DESCRIPTION OF THE DRAWINGS

One or more embodiments of the invention will now be described, by way of example only, with reference to the accompanying drawings, in which:

FIG. 1 shows on top part of the human HFE target RNA sequence (5′ to 3′; SEQ ID NO:52) with the target adenosine in bold face and the tyrosine codon for position 282 in the human HFE protein underlined. Below the target sequence, the sequences (also 5′ to 3′) are given of the initial 51 EONs (SEQ ID NO:1 to 51, in order from top to bottom, respectively) that were designed for editing the target adenosine. The chemical modifications in the EONs are as follows: m5Ue is 2′-MOE modified 5-methyl-uridine; Ge is 2′-MOE modified guanosine; m5Ce is 2′-MOE modified 5-methyl-cytidine; Gm, Am, Um, and Cm are 2′-OMe modified guanosine, adenosine, uridine, and cytidine, respectively; Af, Uf, Gf, and Cf are 2′-F modified adenosine, uridine, guanosine, and cytosine, respectively; Zd is a cytidine analog that is also referred to as a nucleoside carrying a Benner's base (as further outlined herein), with a deoxy moiety (=DNA) at the 2′ ribose position; C2f is a 2′,2′-difluoro modified cytidine; Ad and Cd are deoxyadenosine and deoxycytidine, respectively; an asterisk “*” refers to a phosphorothioate (PS) linkage; a “!” refers to a PNdmi linkage; “\hat{ }” refers to a methylphosphonate (MP) linkage. All other linkages are phosphodiester linkages.

FIG. 2 shows the editing efficiencies over time using EONs RM4700 to RM4726 (as indicated) in an in vitro biochemical editing assay, divided over three panels (A), (B), and (C) for visibility purposes.

FIG. 3 shows editing percentages, determined after 72 hrs of exposure to 5 μM EONs RM4700 to RM4723 and RM4725 (as indicated) in the presence of 1 μM saponin AG1856, each separately in EBV-immortalized B-lymphocytes from a donor (GM14715) homozygous for the C282Y (c.845G>A) mutation in the HFE gene. Negative controls were the use of a scrambled oligonucleotide, a non-treated (NT) sample, a sample for which no reverse transcriptase (−RT) was used, and a water control.

FIG. 4 shows editing percentages, determined after 72 hrs of exposure to 5 μM EONs RM4700 to RM4723 and RM4725 (as indicated) in the presence of 1 μM saponin AG1856, each separately in EBV-immortalized B-lymphocytes from a donor (GM14631) homozygous for the C282Y (c.845G>A) mutation in the HFE gene. Negative controls were the use of a scrambled oligonucleotide, a non-treated (NT) sample, a sample that was only treated with the AG1856 saponin, a sample for which no reverse transcriptase (−RT) was used, and a water control.

FIG. 5 shows on top part of the human HFE target RNA sequence (5′ to 3′; SEQ ID NO:52) with the target adenosine in bold face and the tyrosine codon underlined. Below the target sequence, the sequences (also 5′ to 3′) are given of 49 further EONs (SEQ ID NO:66 to 164) that were designed on top of those depicted in FIG. 1, for editing the target adenosine. The chemical modifications in the EONs are as given for FIG. 1, wherein Gd is deoxyguanosine, Ae is 2′-MOE modified adenosine, and L004 is a 3′-attached tri-antennary GalNAc moiety as described in WO2022/271806.

FIG. 6 shows editing percentages (y-axis left black bars), determined after 72 hrs of exposure of the EONs as depicted below the graph, in a concentration of 5 μM, in the presence of 2 μM saponin AG1856, each separately in EBV-immortalized B-lymphocytes from a donor (GM14715) homozygous for the C282Y (c.845G>A) mutation in the HFE gene. Negative controls were a non-treated (NT) sample and saponin only. RM4717 is a positive control EON taken from FIGS. 1 and 3 (HFE-34). Hepcidin expression levels in these cells upon EON treatment were determined in the same samples and are depicted on the y-axis, right, open bars.

### DETAILED DESCRIPTION

The disclosure describes another approach that is possible to target the p.Cys282Tyr (C282Y) mutation in HFE to generate a wild type HFE protein and potentially restore proper iron processing, and thereby to prevent, ameliorate or treat hereditary HH. This technology is generally referred to as RNA editing. Herein, disclosed are oligonucleotides that can be used to specifically deaminate a specific target adenosine in the transcript of the (human) HFE transcript (pre-mRNA and/or mRNA) in vivo, preferably using endogenous deaminating enzymes, to produce a HFE protein that is restored in its function in hepcidin regulation. By far, the most common mutation that is found in the HFE gene and that in a homozygous genotype may be the cause of iron overload is the C282Y mutation mentioned above, but the RNA editing technology as disclosed herein is also applicable to other target adenosines within HFE that may be targeted to either restore its function or even to cause a gain-of-function effect.

RNA editing is a natural process through which eukaryotic cells alter the sequence of their RNA molecules, often in a site-specific and precise way, thereby increasing the repertoire of genome encoded RNAs by several orders of magnitude. RNA editing enzymes have been described for eukaryotic species throughout the animal and plant kingdoms, and these processes play an important role in managing cellular homeostasis in metazoans from the simplest life forms (such as Caenorhabditis elegans) to humans. Examples of RNA editing are adenosine (A) to inosine (1) conversions and cytidine (C) to uridine (U) conversions, which occur through enzymes called Adenosine Deaminases acting on RNA (ADAR) and APOBEC/AID (cytidine deaminases that act on RNA), respectively.

ADAR is a multi-domain protein, comprising a catalytic domain, and two to three double-stranded RNA recognition domains, depending on the enzyme in question. Each recognition domain recognizes a specific double stranded RNA (dsRNA) sequence and/or conformation. The catalytic domain does also play a role in recognizing and binding a part of the dsRNA helix, although the key function of the catalytic domain is to convert an A into I in a nearby, predefined, position in the target RNA, by deamination of the nucleobase. Inosine is read as guanosine by the translational machinery of the cell, meaning that, if an edited adenosine is in a coding region of an mRNA or pre-mRNA, it can recode the protein sequence. A to I conversions may also occur in 5′ non-coding sequences of a target mRNA, creating new translational start sites upstream of the original start site, which gives rise to N-terminally extended proteins, or in the 3′ UTR or other non-coding parts of the transcript, which may affect the processing and/or stability of the RNA. In addition, A to I conversions may take place in splice elements in introns or exons in pre-mRNAs, thereby altering the pattern of splicing. As a result, exons may be included or skipped. The enzymes catalysing adenosine deamination are within an enzyme family of ADARs, which include human deaminases hADAR1 and hADAR2, as well as hADAR3. However, for hADAR3 no deaminase activity has been demonstrated.

The use of oligonucleotides to edit a target RNA applying adenosine deaminase has been described (e.g., Woolf et al. 1995. PNAS 92:8298-8302; Montiel-Gonzalez et al. 2013. PNAS 110(45):18285-18290; Vogel et al. 2014. Angewandte Chemie Int Ed 53:267-271). A disadvantage of the method described by Montiel-Gonzalez et al. (2013), supra, is the need for a fusion protein consisting of the boxB recognition domain of bacteriophage lambda N-protein, genetically fused to the adenosine deaminase domain of a truncated natural ADAR protein. It requires target cells to be either transduced with the fusion protein, which is a major hurdle, or transfected with a nucleic acid construct encoding the engineered adenosine deaminase fusion protein for expression. The system described by Vogel et al. (2014), supra, suffers from similar drawbacks, in that it is not clear how to apply the system without having to genetically modify the ADAR first and subsequently transfect or transform the cells harboring the target RNA, to provide the cells with this genetically engineered protein. U.S. Pat. No. 9,650,627 describes a similar system. The oligonucleotides of Woolf et al. (1995), supra, that were 100% complementary to the target RNA sequences suffered from severe lack of specificity: nearly all adenosines in the target RNA strand that was complementary to the antisense oligonucleotide were edited.

It is known that ADAR may act on any dsRNA. Through a process sometimes referred to as ‘promiscuous editing’, the enzyme will edit multiple A's in the dsRNA. Hence, there was a need for methods and means that circumvent such promiscuous editing and only target specific adenosines in a target RNA molecule to become therapeutic applicable. Vogel et al. (2014), supra, showed that such off-target editing can be suppressed by using 2′-O-methyl (2′-OMe)-modified nucleosides in the oligonucleotide at positions opposite to adenosines that should not be edited and used a non-modified nucleoside directly opposite to the specifically targeted adenosine on the target RNA. However, the specific editing effect at the target nucleotide has not been shown to take place without the use of recombinant ADAR enzymes having covalent bonds with the AON. Several publications have now shown that the recruitment of endogenous ADAR (hence without the need for an exogenous and/or recombinant source) is feasible while maintaining a specificity in which a single adenosine within a target RNA molecule can be targeted and deaminated to an inosine. WO2016/097212 discloses antisense oligonucleotides (AONs) for the targeted editing of RNA, wherein the AONs are characterized by a sequence that is complementary to a target RNA sequence (therein referred to as the ‘targeting portion’) and by the presence of a stem-loop/hairpin structure (therein referred to as the ‘recruitment portion’), which is preferably non-complementary to the target RNA. Such oligonucleotides are referred to as ‘self-looping AONs’. The recruitment portion acts in recruiting a natural ADAR enzyme present in the cell to the dsRNA formed by hybridization of the target sequence with the targeting portion. Due to the recruitment portion, there is no need for conjugated entities or presence of modified recombinant ADAR enzymes. WO2016/097212 describes the recruitment portion as being a stem-loop structure mimicking either a natural substrate (e.g., the GluB receptor) or a Z-DNA structure known to be recognized by the dsRNA binding domains, or Z-DNA binding domains, of ADAR enzymes. A stem-loop structure can be an intermolecular stem-loop structure, formed by two separate nucleic acid strands, or an intramolecular stem loop structure, formed within a single nucleic acid strand. The stem-loop structure of the recruitment portion as described is an intramolecular stem-loop structure, formed within the AON itself, and are thought to attract (endogenous) ADAR. Similar stem-loop structure-comprising systems for RNA editing have been described in WO2017/050306, WO2020/001793, WO2017/010556, WO2020/246560, and WO2022/078995.

WO2017/220751 and WO2018/041973 describe a next generation type of AONs that do not comprise such a stem-loop structure but that are (almost fully) complementary to the targeted area. In one embodiment, one or more mismatching nucleotides, wobbles, or bulges exist between the oligonucleotide and the target sequence. A sole mismatch may be at the site of the nucleoside opposite the target adenosine, but in other embodiments AONs (or RNA editing oligonucleotides, abbreviated to ‘EONs’) were described with multiple bulges and/or wobbles when attached to the target sequence area. It appeared possible to achieve in vitro, ex vivo and in vivo RNA editing with EONs lacking a stem-loop structure and with endogenous ADAR enzymes when the sequence of the EON was carefully selected such that it could attract/recruit ADAR. The ‘orphan nucleoside’, which is defined as the nucleoside in the EON that is positioned directly opposite the target adenosine in the target RNA molecule, did not carry a 2′-OMe modification. The orphan nucleoside can be a deoxyribonucleoside (DNA), wherein the remainder of the EON could still carry 2′-O-alkyl modifications at the sugar entity (such as 2′-OMe), or the nucleotides directly surrounding the orphan nucleoside contained chemical modifications (such as DNA in comparison to RNA) that further improved the RNA editing efficiency and/or increased the resistance against nucleases. Such effects could even be further improved by using sense oligonucleotides (SONs) that ‘protected’ the EONs against breakdown (described in WO2018/134301). The use of chemical modifications and particular structures in oligonucleotides that could be used in ADAR-mediated editing of specific adenosines in a target RNA have been the subject of numerous publications in the field, such as WO2019/111957, WO2019/158475, WO2020/165077, WO2020/201406, WO2020/211780, WO2021/008447, WO2021/020550, WO2021/060527, WO2021/117729, WO2021/136408, WO2021/182474, WO2021/216853, WO2021/242778, WO2021/242870, WO2021/242889, WO2022/007803, WO2022/018207, WO2022/026928, and WO2022/124345. The use of specific sugar moieties has been disclosed in for instance WO2020/154342, WO2020/154343, WO2020/154344, WO2022/103839, and WO2022/103852, whereas the use of stereo-defined linker moieties (in general for oligonucleotides that for instance can be used for exon skipping, in gapmers, in siRNA, or specifically for RNA-editing oligonucleotides, related to a wide variety of target sequences) has been described in WO2011/005761, WO2014/010250, WO2014/012081, WO2015/107425, WO2017/015575 (HTT), WO2017/062862, WO2017/160741, WO2017/192664, WO2017/192679 (DMD), WO2017/198775, WO2017/210647, WO2018/067973, WO2018/098264, WO2018/223056 (PNPLA3), WO2018/223073 (APOC3), WO2018/223081 (PNPLA3), WO2018/237194, WO2019/032607 (C9orf72), WO2019/055951, WO2019/075357 (SMA/ALS), WO2019/200185 (DM1), WO2019/217784 (DM1), WO2019/219581, WO2020/118246 (DM1), WO2020/160336 (HTT), WO2020/191252, WO2020/196662, WO2020/219981 (USH2A), WO2020/219983 (RHO), WO2020/227691 (C9orf72), WO2021/071788 (C9orf72), WO2021/071858, WO2021/178237 (MAPT), WO2021/234459, WO2021/237223, and WO2022/099159. Next to these disclosures, an extensive number of publications relate to the targeting of specific RNA target molecules, or specific adenosines within such RNA target molecules, be it to repair a mutation that resulted in a premature stop codon, or other mutation causing disease. Examples of such disclosures in which adenosines are targeted within specified target RNA molecules are WO2020/157008 and WO2021/136404 (USH2A); WO2021/113270 (APP); WO2021/113390 (CMT1A); WO2021/209010 (IDUA, Hurler syndrome); WO2021/231673 and WO2021/242903 (LRRK2); WO2021/231675 (ASS1); WO2021/231679 (GJB2); WO2019/071274 and WO2021/231680 (MECP2); WO2021/231685 and WO2021/231692 (OTOF, autosomal recessive non-syndromic hearing loss); WO2021/231691 (XLRS); WO2021/231698 (argininosuccinate lyase deficiency); WO2021/130313 and WO2021/231830 (ABCA4); and WO2021/243023 (SERPINA1).

Disclosed herein are EONs that can produce (or ‘trigger’) RNA editing of a target adenosine in the human HFE transcript (pre-mRNA and/or mRNA), through which the resulting HFE protein is restored in its wild-type function, for instance in hepcidin regulation. In a preferred aspect, the EON causes the deamination of the adenosine present at position 845 of the mutated mRNA, thereby generating an inosine. In other words, the UAC codon encoding tyrosine (mutant form) at amino acid position 282 is converted to a UIC codon, which is read as UGC by the translation machinery, and encodes cysteine (wild-type form). In another embodiment, an EON herein causes the deamination of another adenosine present in the HFE transcript, which may be any adenosine that, when deaminated into an inosine, results in a HFE protein with a gain-of-function. Other mutations may be present in the HFE gene (and transcript), that may be targeted through RNA editing thereby restoring the normal HFE function. As disclosed herein, a preferred mutation that is targeted is the c.845G>A mutation in the human HFE gene resulting in the p.Cys282Tyr HFE protein mutation.

In a preferred embodiment, the EON herein is a single-stranded (ss) oligonucleotide comprising an “orphan nucleotide” that is positioned opposite the target adenosine, wherein the orphan nucleotide is chemically modified as disclosed herein, and wherein the remainder of the oligonucleotide is chemically modified to prevent it from nuclease breakdown also as disclosed herein. In another embodiment, an EON herein relates to any kind of oligonucleotide or heteroduplex oligonucleotide complex, that may or may not be bound to hairpin structures (internally or at the terminal end(s)), that may be bound to ADAR or catalytic domains thereof, or wherein the oligonucleotide is expressed through a vector, such as an AAV, or wherein the oligonucleotide is in a circular format. It is to be understood that any kind of oligonucleotide-based RNA editing is encompassed by the present invention if it relates to the deamination of a nucleotide in the HFE transcript, preferably the mutation causing C282Y, and causes the restoration of the HFE function.

In a preferred aspect, the EON herein is a ‘naked’ oligonucleotide, comprising a variety of chemical modifications in the ribose sugar, the base, and/or the internucleoside linkage of one or more of the nucleotides within the sequence, that can hybridize to the HFE transcript or a part thereof that includes the target adenosine, and can recruit endogenous ADAR for the deamination of the target adenosine.

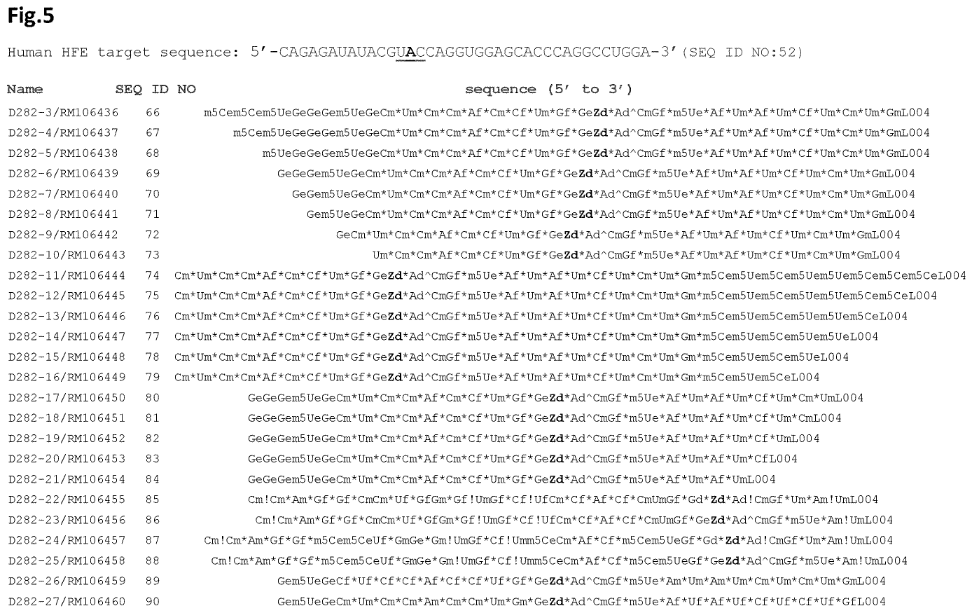

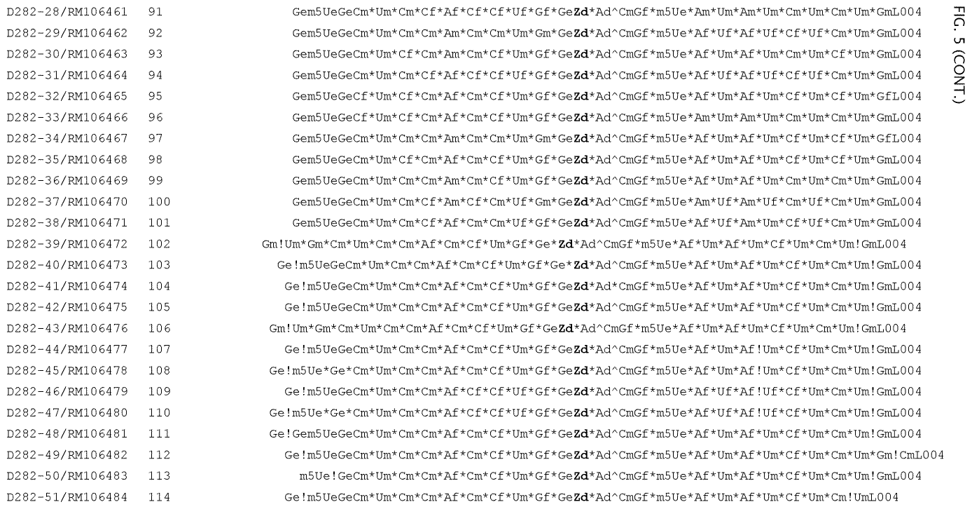

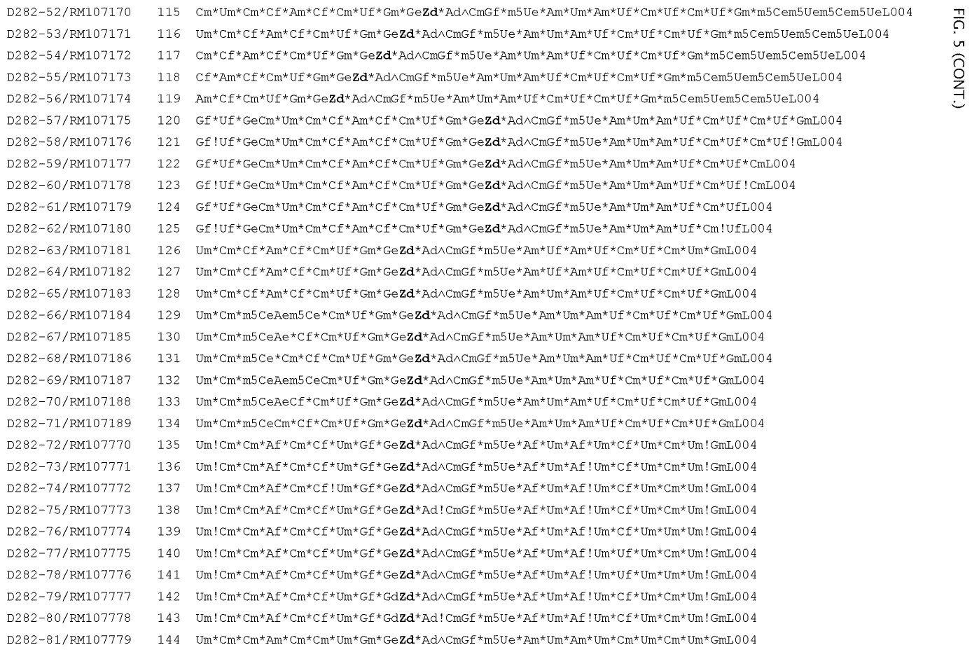

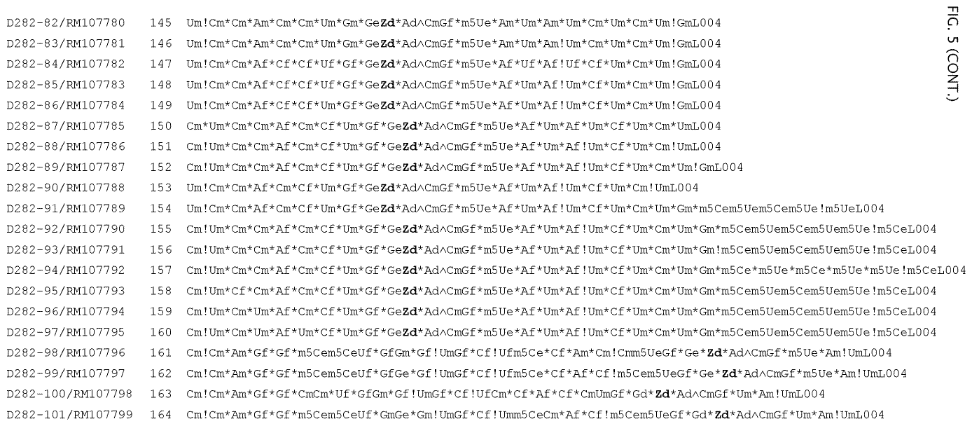

Notably, when the EON comprises chemical modifications, as detailed herein, it may still be delivered through the means of a delivery vehicle. Suitable delivery vehicles are for instance Lipid Nanoparticles (LNP's) that are nano-sized lipid vesicles that carry the EON herein and aid to the delivery of target cells. If an LNP is applied or any other similar type of carrier, the EON is still considered naked because it is not transcribed from an encoding polynucleotide (such as in the case of a plasmid or a vector, in which the EON is not regarded as ‘naked’, but transcribed). So, even though a chemically modified AON is encapsulated by a carrier, preferably an LNP, it is still seen as naked, as it has been manufactured as such in a laboratory setting and encapsulated thereafter in the carrier using methods known to the person skilled in the art. The disclosure also relates to a delivery vehicle, preferably an LNP, which comprises a chemically modified AON as disclosed herein, even more preferably as disclosed in any one of SEQ ID NO:1 to 51 and 66 to 164. The person skilled in the art understands that when a delivery moiety, or attachment to the EON is used (such a GalNAc moiety to target hepatocytes in the liver, e.g., the L004 GalNAc moiety as shown in FIG. 5) that the EON is still seen as naked as well, also when a GalNAc-EON is encapsulated in a delivery vehicle such as an LNP.

### EMBODIMENTS

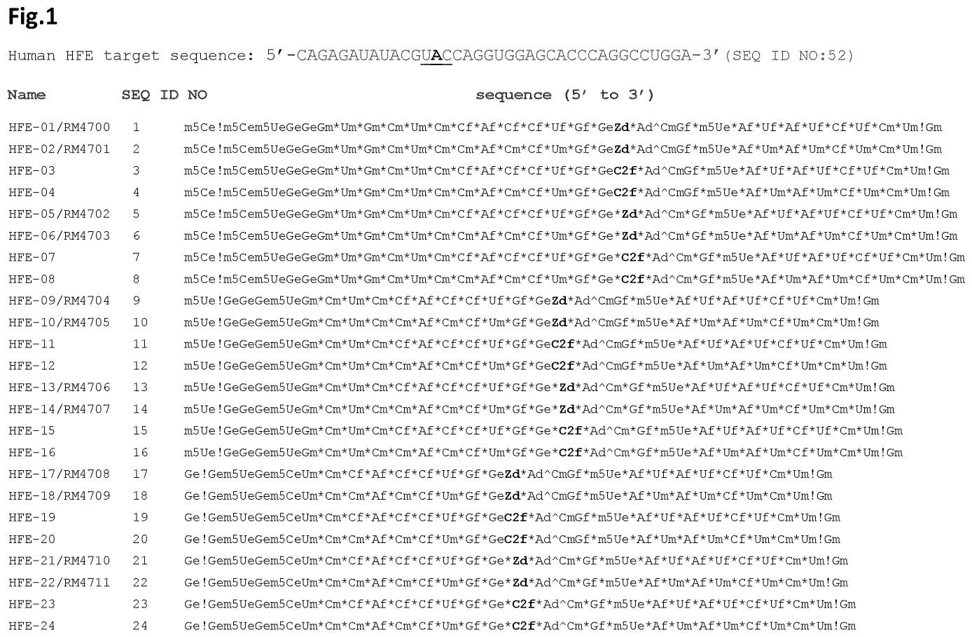

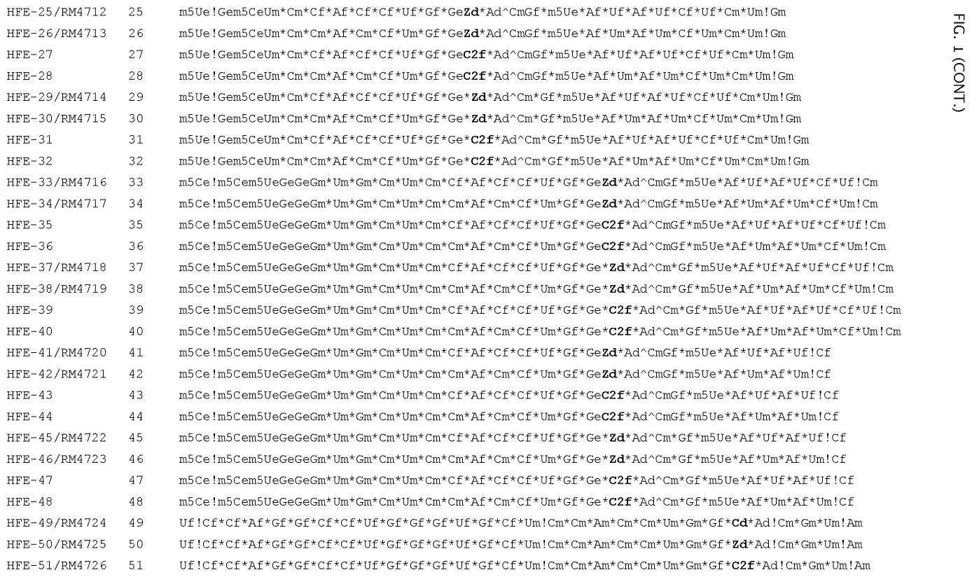

The disclosure provides an EON capable of forming a ds complex with a region of an endogenous human HFE transcript molecule in a cell, wherein the region of the HFE transcript molecule comprises a target adenosine, and wherein the ds complex can recruit an endogenous ADAR enzyme to deaminate the target adenosine into an inosine, thereby editing the HFE transcript molecule. In a preferred aspect, the HFE transcript molecule is a pre-mRNA or an mRNA molecule. In one embodiment, the cell is a human liver cell, preferably a hepatocyte. In one embodiment, the target adenosine is a c.845G>A mutation in the HFE gene. In one embodiment, the deamination of the target adenosine results in restoration of a wild-type HFE protein, although this is not necessarily the case if the transcript comprises mutations other than the c.845G>A mutation. In one embodiment, the EON comprises or consists of the nucleotide sequence of any one of the EON sequences depicted in FIG. 1 or 5, with alternative chemical modifications as outlined in detail herein. In one embodiment, the respective EON comprises the chemical modifications as depicted in FIG. 1 or 5. In one embodiment, at least one nucleotide comprises one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution. In one embodiment, the orphan nucleotide is a cytidine analog such as a deoxynucleotide comprising a 6-amino-5-nitro-3-yl-2(1H)-pyridone nucleobase (also known as Benner's base). In one embodiment, the orphan nucleotide is a uridine analog such as a deoxynucleotide comprising an iso-uracil nucleobase. In one embodiment, the EON comprises one or more mismatches, wobbles, or bulges, wherein a single mismatch may be present when the target adenosine has an opposite cytidine in the EON. If the orphan nucleotide is a cytidine, it does not comprise a 2′-OMe ribose substitution. In one embodiment, the one or more additional modifications in the linkage moiety is each independently selected from a PS, phosphonoacetate, phosphorodithioate, MP, sulfonylphosphoramidate, or PNdmi internucleotide linkage. In one embodiment, the one or more additional modifications in the ribose moiety is a mono- or di-substitution at the 2′, 3′ and/or 5′ position of the ribose, each independently selected from the group consisting of: —OH; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

In one embodiment, the disclosure provides a vector, preferably a viral vector, more preferably an AAV vector, comprising a nucleic acid molecule encoding an EON herein. In an embodiment, the disclosure provides a pharmaceutical composition comprising an EON herein or a vector herein, and a pharmaceutically acceptable carrier. In one embodiment, the disclosure provides an EON herein, a vector herein, an LNP formulation herein, or a pharmaceutical composition herein for use in the treatment of an iron overload disorder, preferably HFE hemochromatosis. In one embodiment, the disclosure provides use of an EON herein, a vector herein, or an LNP formulation herein in the manufacture of a medicament for the treatment of a disorder related to iron overload, preferably HFE hemochromatosis.

In one embodiment, the disclosure provides a method of editing a HFE polynucleotide, the method comprising contacting the HFE polynucleotide with an EON capable of effecting an ADAR-mediated adenosine to inosine alteration of an adenosine associated with iron homeostasis, thereby editing the HFE polynucleotide, preferably wherein the EON is as disclosed herein. In one embodiment, the provides a method of treating HFE hemochromatosis in a patient in need thereof, the method comprising contacting a HFE polynucleotide in a cell of the subject with an EON capable of effecting an ADAR-mediated adenosine to inosine alteration of an adenosine associated with iron homeostasis, preferably wherein the EON is as disclosed herein, or a vector encoding an EON herein, or an LNP formulation herein, thereby treating the patient. In one embodiment, the disclosure provides a method of treating HFE hemochromatosis, the method comprising administering to a patient in need thereof a therapeutically effective amount of an EON herein, a vector herein, or a pharmaceutical composition herein.

In one embodiment, the disclosure provides a method of deamidating a target adenosine in an HFE pre-mRNA or mRNA molecule in a cell, the method comprising the steps of: (i) providing the cell with an EON, an LNP formulation, or a vector as disclosed herein; (ii) allowing uptake by the cell of the EON, the LNP, or vector, respectively; (iii) allowing annealing of the EON to the HFE pre-mRNA or mRNA molecule; (iv) allowing an endogenous ADAR enzyme to deaminate the target adenosine in the target RNA molecule to an inosine; and optionally (v) identifying the presence of the inosine in the target RNA molecule. In a preferred aspect, the target adenosine is a c.845G>A mutation in a human HFE pre-mRNA or mRNA molecule. The step of identifying the presence of an inosine at the position of the target adenosine preferably comprises: (a) determining the sequence of the HFE pre-mRNA or mRNA molecule; (b) assessing the presence of a wild-type HFE protein; or (c) using a functional read-out, preferably assessing a serum or plasma ferritin concentration, or a serum transferrin saturation percentage. Such assessments can be performed in vitro on samples taken from the treated subject. For example, measuring the ferritin concentrations can be assessed before and after EON treatment to determine the level of EON activity (and of course, RNA editing of the target transcript).

### Definitions

The term ‘nucleoside’ refers to the nucleobase linked to the (deoxy)ribosyl sugar, without phosphate groups. A ‘nucleotide’ is composed of a nucleoside and one or more phosphate groups. The term ‘nucleotide’ thus refers to the respective nucleobase-(deoxy)ribosyl-phospholinker, as well as any chemical modifications of the ribose moiety or the phospho group. Thus, the term would include a nucleotide including a locked ribosyl moiety (comprising a 2′-4′ bridge, comprising a methylene group or any other group), an unlocked nucleic acid (UNA), a threose nucleic acid (TNA), a nucleotide including a linker comprising a phosphodiester, phosphonoacetate, phosphotriester, PS, phosphoro(di)thioate, MP, methyl thiophosphonate, phosphoramidate linkages, and the like. Sometimes the terms adenosine and adenine, guanosine and guanine, cytidine and cytosine, uracil and uridine, thymine and thymidine/uridine, inosine, and hypoxanthine, are used interchangeably to refer to the corresponding nucleobase on the one hand, and the nucleoside or nucleotide on the other. Thymine (T) is also known as 5-methyluracil (m⁵U) and is a uracil (U) derivative; thymine, 5-methyluracil and uracil can be interchanged throughout the document text. Likewise, thymidine is also known as 5-methyluridine and is a uridine derivative; thymidine, 5-methyluridine and uridine can be interchanged throughout the document text. Sometimes the terms nucleobase, nucleoside and nucleotide are used interchangeably, unless the context clearly requires differently, for instance when a nucleoside is linked to a neighbouring nucleoside and the linkage between these nucleosides is modified. As stated herein, a nucleotide is a nucleoside plus one or more phosphate groups. The terms ‘ribonucleoside’ and ‘deoxyribonucleoside’, or ‘ribose’ and ‘deoxyribose’ are as used in the art.

Whenever reference is made to an oligonucleotide, oligo, ON, ASO, oligonucleotide composition, antisense oligonucleotide, AON, (RNA) editing oligonucleotide, EON, and RNA (antisense) oligonucleotide, both oligoribonucleotides and deoxyoligoribonucleotides are meant unless the context dictates otherwise. Potentially the oligonucleotide may completely lack RNA or DNA nucleotides (as they appear in nature) and may consist completely of modified nucleotides. Whenever reference is made to an ‘oligoribonucleotide’ it may comprise the bases A, G, C, U, or I. Whenever reference is made to a ‘deoxyoligoribonucleotide’ it may comprise the bases A, G, C, T, or I. However, an oligonucleotide of the present invention may comprise a mix of ribonucleosides and deoxyribonucleosides. When a deoxyribonucleotide is used, hence without a modification at the 2′ position of the sugar, the nucleotide is often abbreviated to dA. dC, dG or T in which the ‘d’ represents the deoxy nature of the nucleoside, while a ribonucleoside that is either normal RNA or modified at the 2′ position is often abbreviated without the ‘d’, and often abbreviated with their respective modifications and as explained herein.

Whenever reference is made to nucleotides in the oligonucleotide, such as cytosine, 5-methylcytosine, 5-hydroxymethylcytosine, 5-formylcytosine, 5-acetylcytosine, 5-hydroxycytosine, and β-D-glucosyl-5-hydroxymethylcytosine are included. Whenever reference is made to adenine, N6-methyladenine, 8-oxo-adenine, 2,6-diaminopurine and 7-methyladenine are included. Whenever reference is made to uracil, dihydrouracil, isouracil, N3-glycosylated uracil, pseudouracil, 5-methyluracil, N1-methylpseudouracil, 4-thiouracil and 5-hydroxymethyluracil are included. Whenever reference is made to guanine, 1-methylguanine, 7-methylguanosine, N2,N2-dimethylguanosine, N2,N2,7-trimethylguanosine and N2,7-dimethylguanosine are included. Whenever reference is made to nucleosides or nucleotides, ribofuranose derivatives, such as 2′-deoxy, 2′-hydroxy, and 2′-O-substituted variants, such as 2′-OMe, are included, as well as other modifications, including 2′-4′ bridged variants. Whenever reference is made to oligonucleotides, linkages between two mononucleotides may be phosphodiester linkages as well as modifications thereof, including, phosphonoacetate, phosphotriester, PS, phosphoro(di)thioate, MP, phosphoramidate linkers, phosphoryl guanidine, thiophosphoryl guanidine, sulfono phosphoramidate and the like.

The term ‘comprising’ encompasses ‘including’ as well as ‘consisting of’, e.g., a composition ‘comprising X’ may consist exclusively of X or may include something additional, e.g., X+Y. The term ‘about’ in relation to a numerical value x is optional and means, e.g., x±10%.

The word ‘substantially’ does not exclude ‘completely’, e.g., a composition which is ‘substantially free from Y’ may be completely free from Y. Where relevant, the word ‘substantially’ may be omitted from the definition of the invention.

The term ‘complementary’ as used herein refers to the fact that the EON hybridizes under physiological conditions to a second nucleic acid strand (for instance when the oligonucleotide as a first nucleic acid strand (=guide oligonucleotide) forms a heteroduplex RNA editing oligonucleotide complex, or HEON, with another complementary nucleic acid strand), or when it forms a double stranded complex with the target RNA sequence. The term does not necessarily mean that each nucleotide in a nucleic acid strand has a perfect pairing with its opposite nucleotide in the opposite sequence. In other words, while an EON may be complementary to a target sequence, there may be mismatches, wobbles and/or bulges between the oligonucleotide and the target sequence, while under physiological conditions that EON still hybridizes to the target sequence such that the cellular RNA editing enzymes can edit the target adenosine. The term ‘substantially complementary’ therefore also means that despite the presence of the mismatches, wobbles, and/or bulges, the EON has enough matching nucleotides between the EON and target sequence that under physiological conditions the EON hybridizes to the target RNA. As shown herein, an EON may be complementary, but may also comprise one or more mismatches, wobbles and/or bulges with the target sequence, if under physiological conditions the EON is able to hybridize to its target.

The term ‘downstream’ in relation to a nucleic acid sequence means further along the sequence in the 3′ direction; the term ‘upstream’ means the converse. Thus, in any sequence encoding a polypeptide, the start codon is upstream of the stop codon in the sense strand but is downstream of the stop codon in the antisense strand.

References to ‘hybridisation’ typically refer to specific hybridisation and exclude non-specific hybridisation. Specific hybridisation can occur under experimental conditions chosen, using techniques well known in the art, to ensure that most stable interactions between probe and target are where the probe and target have at least 70%, preferably at least 80%, more preferably at least 90% sequence identity.

The term ‘mismatch’ is used herein to refer to opposing nucleotides in a double stranded RNA complex which do not form perfect base pairs according to the Watson-Crick base pairing rules. In the historical sense, mismatched nucleotides are G-A, C-A, U-C, A-A, G-G, C-C, U-U pairs. In some embodiments the EON comprises fewer than four mismatches with the target sequence, for example 0, 1 or 2 mismatches. ‘Wobble’ base pairs are G-U, I-U, I-A, and I-C base pairs. Although a G:G pairing would be considered a mismatch, that does not necessarily mean that the interaction is unstable, which means that the term ‘mismatch’ may be somewhat outdated based on the current invention where a Hoogsteen base-pairing may be seen as a mismatch based on the origin of the nucleotide but still be relatively stable. An isolated G:G pairing in duplex RNA can for instance be quite stable, but still be defined as a mismatch.

The term ‘splice mutation’ relates to a mutation in a gene that encodes for a pre-mRNA, wherein the splicing machinery is dysfunctional in the sense that splicing of introns from exons is disturbed and due to the aberrant splicing, the subsequent translation is out of frame resulting in premature termination of the encoded protein. Often such shortened proteins are degraded rapidly and do not have any functional activity.

An EON (and the complementary nucleic acid strand when two oligonucleotides form a HEON) herein may be chemically modified almost in its entirety, for example by providing nucleotides with a ribose sugar moiety carrying a 2′-OMe substitution, a 2′-F substitution, or a 2′-O-methoxyethyl (2′-MOE) substitution. The orphan nucleotide in the EON is preferably a cytidine or analog thereof (such as a nucleotide carrying a Benner's base), or a uridine or analog thereof (such as iso-uridine), and/or in one embodiment comprises a diF modification at the 2′ position of the sugar, in another embodiment comprises a deoxyribose (2′-H, DNA), and in yet a further embodiment, at least one and in another embodiment both the two neighbouring nucleotides flanking the orphan nucleotide do not comprise a 2′-OMe modification. Complete modification wherein all nucleotides of the oligonucleotide hold a 2′-OMe modification, with natural bases, results in a non-functional oligonucleotide as far as RNA editing goes (known in the art), presumably because it hinders the ADAR activity at the targeted position. In general, an adenosine in a target RNA can be protected from editing by providing an opposing nucleotide with a 2′-OMe group (at least when there are no other chemical substitutions or modifications within the nucleotide), or by providing a guanine or adenine as opposing base, as these two nucleobases are also able to reduce editing of the opposing adenosine.

Various chemistries and modifications are known in the field of oligonucleotides that can be readily used in accordance with the disclosure. The regular internucleosidic linkages between the nucleotides may be altered by mono- or di-thioation of the phosphodiester bonds to yield PS esters or phosphorodithioate esters, respectively. Other modifications of the internucleosidic linkages are possible, including amidation and peptide linkers.

In an embodiment, the EON herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 nucleotides.

It is known in the art that RNA editing entities (such as human ADAR enzymes) edit dsRNA structures with varying specificity, depending on several factors. One important factor is the degree of complementarity of the two strands making up the dsRNA sequence. Perfect complementarity of the two strands usually causes the catalytic domain of human ADAR to deaminate adenosines in a non-discriminative manner, reacting with any adenosine it encounters. The specificity of hADAR1 and 2 can be increased by introducing chemical modifications and/or ensuring several mismatches in the dsRNA, which presumably helps to position the dsRNA binding domains in a way that has not been clearly defined yet. Additionally, the deamination reaction itself can be enhanced by providing an oligonucleotide that comprises a mismatch opposite the adenosine to be edited. Following the instructions in the present application, those of skill in the art will be capable of designing the complementary portion of the oligonucleotide according to their needs.

The RNA editing protein present in the cell that is of most interest to be used with an EON herein is human ADAR2. It will be understood by a person having ordinary skill in the art that the extent to which the editing entities inside the cell are redirected to other target sites may be regulated by varying the affinity of the first nucleic acid strand for the recognition domain of the editing molecule. The exact modification may be determined through some trial and error and/or through computational methods based on structural interactions between the EON and the recognition domain of the editing molecule. In addition, or alternatively, the degree of recruiting and redirecting the editing entity resident in the cell may be regulated by the dosing and the dosing regimen of the EON. This is something to be determined by the experimenter (in vitro) or the clinician, usually in phase I and/or II clinical trials.

The disclosure also concerns modifying target RNA sequences in eukaryotic, preferably metazoan, more preferably mammalian, most preferably human cells. The disclosure is particularly suitable for modifying RNA sequences in cells and tissues in which HFE is expressed and wherein that protein acts. The pathogenic mechanisms by which the mutated HFE-gene product afflicts the iron homeostasis are not fully understood. Hepcidin, which is produced in the liver, is the ‘master regulator’ of body iron homeostasis, and its main task is to inactivate ferroportin. Ferroportin has an important position in the regulation of iron transport out through the cell membrane (efflux) in enterocytes, hepatocytes, and macrophages. Normal HFE and transferrin receptor-2 complexes on the cell membrane of the hepatocytes stimulate the production/activation of hepcidin, which subsequently inhibits intestinal iron uptake. In hemochromatosis, because of a defective HFE complex, the production/activation of hepcidin is reduced, resulting in an increased intestinal iron uptake, which is largely independent of the body's iron status. HH is thus characterized by a low plasma concentration of hepcidin termed ‘hepcidin insufficiency’. Intracellular iron accumulation triggers oxidative stress, DNA damage, cellular necrosis and over time fibrosis. This development is typically seen in the liver, where initial fibrosis may eventually progress into cirrhosis. Because HFE is predominantly produced and has an important role in liver cells, the preferred target cells for the EONs herein are liver cells, more preferably hepatocytes. The target cell can be located in vitro, ex vivo or in vivo. One advantage of the EONs disclosed herein is that they can be used with cells in situ in a living organism but also can be used with cells in culture. In some embodiments cells are treated ex vivo and are then introduced into a living organism (e.g., re-introduced into an organism from whom they were originally derived). The EONs herein can also be used to edit target RNA sequences in cells from a transplant or within a so-called organoid, e.g., a liver tissue organoid. Organoids can be thought of as three-dimensional in vitro-derived tissues but are driven using specific conditions to generate individual, isolated tissues. In a therapeutic setting they are useful because they can be derived in vitro from a patient's cells, and the organoids can then be re-introduced to the patient as autologous material which is less likely to be rejected than a normal transplant.

Without wishing to be bound by theory, the RNA editing through hADAR2 is thought to take place on primary transcripts in the nucleus, during transcription or splicing, or in the cytoplasm, where e.g., mature mRNA, miRNA or ncRNA can be edited.

It should be clear, that targeted editing according to the disclosure can be applied to any adenosine within the HFE transcript if the deamination of the adenosine results in an increase or restoration of HFE protein function. As outlined herein, it is however preferred to target the adenosine at position 845 (=the c.845G>A mutation) in the mutant HFE transcript product to yield a change from a UAC codon (encoding tyrosine) to UIC (or UGC, encoding cysteine). Generally spoken, RNA editing may be used to create RNA sequences with different properties. Such properties may be coding properties (creating proteins with different sequences or length, leading to altered protein properties or functions), or binding properties (causing inhibition or over-expression of the RNA itself or a target or binding partner; entire expression pathways may be altered by recoding miRNAs or their cognate sequences on target RNAs). Protein function or localization may be changed at will, by functional domains or recognition motifs, including but not limited to signal sequences, targeting or localization signals, recognition sites for proteolytic cleavage or co- or post-translational modification, catalytic sites of enzymes, binding sites for binding partners, signals for degradation or activation and so on. These and other forms of RNA and protein “engineering”, whether to prevent, delay or treat disease or for any other purpose, in medicine or biotechnology, as diagnostic, prophylactic, therapeutic, research tool or otherwise, are encompassed by the present invention. Hence, any RNA editing of a target adenosine in the HFE transcript and that results in improvement or restoration of the HFE protein function is encompassed by the present invention.

The present disclosure opens a whole new field of treating iron overload, or HH, using genetic editing techniques. The genetic editing technique is not particularly limited. Suitable techniques include known gene therapy techniques, which include DNA editing techniques such as CRISPR/Cas, ZFNs, TALENs, and meganucleases, and preferably RNA editing techniques such as ADAR-mediated editing techniques, as further outlined in detail herein.

The amount of EON to be administered, the dosage and the dosing regimen can vary from cell type to cell type, the disease to be treated, the target population, the mode of administration (e.g., systemic versus local), the severity of disease and the acceptable level of side activity, but these can and should be assessed by trial and error during in vitro research, in pre-clinical and clinical trials. The trials are particularly straightforward when the modified sequence leads to an easily detected phenotypic change, or a change in (the level of, or activity of) a specified biomarker. It is possible that higher doses of EONs could compete for binding to an ADAR within a cell, thereby depleting the amount of the entity, which is free to take part in RNA editing, but routine dosing trials will reveal any such effects for a given EON and a given target.

One suitable trial technique involves delivering the EON to cell lines, or a test organism and then taking biopsy samples at various time points thereafter. The sequence of the target RNA can be assessed in the biopsy sample and the proportion of cells having the modification can easily be followed. After this trial has been performed once then the knowledge can be retained, and future delivery can be performed without needing to take biopsy samples. A method of the invention can thus include a step of identifying the presence of the desired change in the cell's target RNA sequence, thereby verifying that the target RNA sequence has been modified. This step will typically involve sequencing of the relevant part of the target RNA, or a cDNA copy thereof (or a cDNA copy of a splicing product thereof, in case the target RNA is a pre-mRNA), as discussed above, and the sequence change can thus be easily verified. Alternatively, the change may be assessed on the function of the protein, for instance by measuring or assessing a serum or plasma ferritin concentration or a serum transferrin saturation percentage before and/or after treatment or assessing any other potential marker, which measurements are preferably performed in vitro on samples obtained from the treated subject.

After RNA editing has occurred in a cell, the modified RNA can become diluted over time, for example due to cell division, limited half-life of the edited RNAs, etc. Thus, in practical therapeutic terms a method of the invention may involve repeated delivery of an EON until enough target RNAs have been modified to provide a tangible benefit to the patient and/or to maintain the benefits over time.

EONs herein are particularly suitable for therapeutic use, and so the disclosure also relates to a pharmaceutical composition comprising an EON herein, or a vector or plasmid encoding the EON herein, and a pharmaceutically acceptable carrier. In some embodiments, the pharmaceutically acceptable carrier can simply be a saline solution. This can usefully be isotonic or hypotonic, particularly for pulmonary delivery. The disclosure also provides a delivery device (e.g., syringe, inhaler, nebuliser) which includes a pharmaceutical composition of the invention.

The disclosure also provides an EON herein for use in a method for repairing a mutation in a target HFE RNA sequence in a mammalian, preferably a human liver cell, as described herein. Similarly, the disclosure provides the use of an EON herein in the manufacture of a medicament for making a change in a target HFE RNA sequence in a mammalian, preferably a human liver cell, as described herein, and thereby treating, preventing, or ameliorating diseases related to iron overload, such as HFE hemochromatosis.

The disclosure also provides a method of deamidating at least one specific target adenosine present in a target HFE RNA sequence in a cell, the method comprising the steps of: providing the cell with an EON herein; allowing uptake by the cell of the EON; allowing annealing of the EON to the target RNA molecule; allowing a mammalian ADAR enzyme comprising a natural dsRNA binding domain as found in the wild type enzyme to deaminate the target adenosine (preferably the adenosine at position 845 in the mutant HFE transcript product) in the target RNA molecule to an inosine; and optionally identifying the presence of the inosine in the RNA sequence.

The disclosure also provides a method of deamidating at least one specific target adenosine present in a target HFE RNA sequence in a cell, the method comprising the steps of: providing the cell with a vector or plasmid encoding the EON herein; allowing uptake by the cell of the vector or plasmid; allowing annealing of the EON to the target RNA molecule; allowing a mammalian ADAR enzyme comprising a natural dsRNA binding domain as found in the wild type enzyme to deaminate the target adenosine (preferably the adenosine at position 845 in the mutant HFE transcript product) in the target RNA molecule to an inosine; and optionally identifying the presence of the inosine in the RNA sequence.

In a preferred aspect, depending on the ultimate deamination effect of A to I conversion, the identification step comprises the following steps: sequencing the target RNA; assessing the presence or absence of a functional protein; assessing whether splicing of the pre-mRNA was altered by the deamination; or using a functional read-out, because the target RNA after the deamination should encode a functional protein. Examples are assessing ferritin or hepcidin concentrations after RNA editing. The ferritin concentration is generally regarded as the best biomarker for the body's iron content. The serum transferrin saturation percentage is an indicator of the iron content of the blood and the iron supply to the organs. High serum transferrin saturation is usually the first indicator of HFE hemochromatosis and can be present, even though serum ferritin is still within the normal range. The identification of the deamination into inosine may therefore be a functional read-out using a suitable biomarker. The functional assessment for HFE hemochromatosis mentioned herein will generally be according to methods known to the skilled person. A very suitable manner to identify the presence of an inosine after deamination of the target adenosine is of course dPCR or even sequencing, using methods that are well-known to the person skilled in the art. However, the person skilled in the art of liver disease may apply tests to monitor certain biomarkers related to iron overload, as discussed above.

The EON herein is suitably administrated in aqueous solution, e.g. saline, or in suspension, optionally comprising additives, excipients and other ingredients, compatible with pharmaceutical use, at concentrations ranging from 1 ng/ml to 1 g/ml, preferably from 10 ng/ml to 500 mg/ml, more preferably from 100 ng/ml to 100 mg/ml. Dosage may suitably range from between about 1 μg/kg to about 100 mg/kg, preferably from about 10 μg/kg to about 10 mg/kg, more preferably from about 100 μg/kg to about 1 mg/kg. Administration may be by inhalation (e.g., through nebulization), intranasally, orally, by injection or infusion, intravenously, subcutaneously, intradermally, intramuscularly, intra-tracheally, intra-peritoneally, intrarectally, intrathecally, intra-cisterna magna, parenterally, and the like. Administration may be in solid form, in the form of a powder, a pill, a gel, a solution, a slow-release formulation, or in any other form compatible with pharmaceutical use in humans.

In one embodiment, a method herein comprises the steps of administering to the subject an EON or pharmaceutical composition herein, allowing the formation of a ds nucleic acid complex of the EON with its specific complementary target nucleic acid molecule in a cell in the subject; allowing the engagement of an endogenous present adenosine deaminating enzyme, such as ADAR2; and allowing the enzyme to deaminate the target adenosine in the target nucleic target molecule to an inosine, thereby alleviating, preventing or ameliorating the disease related to iron overload. The diseases that may be treated according to this method are preferably, but not limited to, the genetic diseases listed herein, and any other disease in which deamination of an adenosine in HFE transcripts would restore the protein's function in a patient in need thereof.

RNA editing molecules present in the cell will usually be proteinaceous in nature, such as the ADAR enzymes found in metazoans, including mammals. Preferably, the cellular editing entity is an enzyme, more preferably an adenosine deaminase or a cytidine deaminase, still more preferably an adenosine deaminase. These are enzymes with ADAR activity. The ones of most interest are the human ADARs, hADAR1 and hADAR2, including any isoforms thereof. RNA editing enzymes known in the art, for which oligonucleotide constructs according to the invention may conveniently be designed, include the adenosine deaminases acting on RNA (ADARs), such as hADAR1 and hADAR2 in humans or human cells and cytidine deaminases. It is known that hADAR1 exists in two isoforms; a long 150 kDa interferon inducible version and a shorter, 100 kDa version, that is produced through alternative splicing from a common pre-mRNA. Consequently, the level of the 150 kDa isoform available in the cell may be influenced by interferon, particularly interferon-gamma (IFN-γ). hADAR1 is also inducible by TNF-α. This provides an opportunity to develop combination therapy, whereby IFN-γ or TNF-α and EONs according to the invention are administered to a patient either as a combination product, or as separate products, either simultaneously or subsequently, in any order. Certain disease conditions may already coincide with increased IFN-γ or TNF-α levels in certain tissues of a patient, creating further opportunities to make editing more specific for diseased tissues. It will be understood by a person having ordinary skill in the art that the extent to which the editing entities inside the cell are redirected to other target sites may be regulated by varying the affinity of the first nucleic acid strand for the recognition domain of the editing molecule.

### Chemical Modifications

All chemical modifications listed below that may be used in the EON herein may also be used for a sense strand that is complementary to the EON, when the EON and the complementary strand form a so-called heteroduplex RNA editing oligonucleotide (HEON) complex, as described in GB 2215614.5 (unpublished), except that the opposite sense strand does not have an orphan nucleotide. Hence, the modification related to the orphan nucleotide relate only to the EON herein, but all other modifications relate to the EON herein and any (protecting) sense oligonucleotide that may be used together with the EON in a pharmaceutical product. This includes the use of hydrophobic moieties (such as tocopherol and cholesterol) and cell-specific ligands (such as GalNAc moieties), that have also been described herein, and in detail in GB 2215614.5 (unpublished), which may either be bound to the EON or its opposite strand, or both.

The internucleoside linkages in the oligonucleotides herein may comprise one or more naturally occurring internucleoside linkages and/or modified internucleoside linkages. Without limitations, at least one, at least two, or at least three internucleoside linkages from a 5′ and/or 3′ end of the EON are preferably modified internucleoside linkages. A preferred modified internucleoside linkage is a PS linkage. In one embodiment, all internucleoside linkages of the EON are modified internucleoside linkages. In one embodiment, the EON comprises a PNdmi linkage linking the most terminal nucleoside at the 5′ and/or 3′ end, and the one before last nucleoside at each of these ends, respectively. A PNdmi linkage as preferably used in the EONs herein has the structure of the following formula:

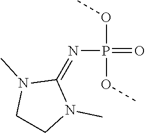

PNdmi
linkage

A common limiting factor in oligonucleotide-based therapies are the oligonucleotide's ability to be taken up by the cell (when delivered per se, or ‘naked’ without applying a delivery vehicle), its biodistribution and its resistance to nuclease-mediated breakdown. The skilled person is aware, and it has been described in detail in the art, that a variety of chemical modifications can assist in overcoming such limitations. Examples of such now commonly used chemical modifications are the 2′-O-methyl (often abbreviated to 2′-OMe or 2′-O-Me), 2′-F and 2′-O-methoxyethyl (often also referred to as 2′-methoxyethoxy, or 2′-MOE) modifications of the sugar and the use of PS linkages between nucleosides. WO2020/201406 discloses the use of MP linkage modifications at certain positions surrounding the orphan nucleotide in the first nucleic acid strand. The ribose 2′ groups in all nucleotides of the EON, except for the ribose sugar moiety of the orphan nucleotide that has certain limitations in respect of compatibility with RNA editing, can be independently selected from 2′-H (i.e., DNA), 2′-OH (i.e., RNA), 2′-OMe, 2′-MOE, 2′-F, or 2′-4′-linked (for instance a locked nucleic acid (LNA)), or other ribosyl 1′-substitutions, 2′ substitutions, 3′ substitutions, 4′ substitutions or 5′ substitutions. The orphan nucleotide in the EON that comprises no other chemical modifications to the ribose sugar, the base, or the linkage preferably does not carry a 2′-OMe or 2′-MOE substitution but may carry a 2′-F, a 2′,2′-difluoro (diF), or 2′-ara-F (FANA) substitution or may be DNA. GB 2214347.3 (unpublished) describes the modification of the 2′ position of the ribose sugar moiety of the orphan nucleotide by a 2′,2′-disubstituted substitution such as diF, which is also applicable to the invention described here. The 2′-4′ linkage can be selected from many linkers known in the art, such as a methylene linker, amide linker, or constrained ethyl linker (cEt).

The disclosure provides an EON for use in the deamination of a target nucleotide (preferably adenosine) in a target RNA, wherein the EON is complementary to a stretch of nucleotides in the target RNA that includes the target adenosine, wherein the nucleotide in the first nucleic acid strand that is directly opposite the target nucleotide is the orphan nucleotide, and when the target nucleotide is an adenosine the orphan nucleotide comprises preferably a base or modified base or base analogue with a NH moiety at the position similar to the ring nitrogen (e.g., Benner's base Z). The nucleotide numbering in the EON is such that the orphan nucleotide is number 0 and the nucleotide 5′ from the orphan nucleotide is α+1. Counting is further positively (+) incremented towards the 5′ end and negatively (−) incremented towards the 3′ end, wherein the first nucleotide 3′ from the orphan nucleotide is number −1. The internucleoside linkage numbering in the EON is such that linkage number 0 is the linkage 5′ from the orphan nucleotide, and the linkage positions in the oligonucleotide are positively (+) incremented towards the 5′ end and negatively (−) incremented towards the 3′ end.

Preferably, the EON comprises one or more (chirally pure or chirally mixed) PS linkages. In one embodiment, the PS linkages connect the terminal 3, 4, 5, 6, 7, or 8 nucleotides on each end of the first nucleic acid strand. In one embodiment, the EON comprises one of more phosphoramidate (PN) linkages. In one embodiment, a PN linkage connects the terminal two nucleotides on each end of the EON.

A nucleoside in the EON may be a natural nucleoside (deoxyribonucleoside or ribonucleoside) or a non-natural nucleoside. It is noted that for RNA editing, in which double-stranded RNA is generally the substrate for enzymes with deamination activity (such as ADARs), ribonucleosides are considered ‘natural’, while deoxyribonucleosides may then be, for the sake of argument, considered as non-natural, or modified, simply because DNA is not present in the RNA-RNA double stranded substrate configurations. The skilled person appreciates that when the nucleotide has a natural ribose moiety, it may still be non-naturally modified in the base and/or the linkage.

In addition to the specific preferred chemical modifications at certain positions in compounds herein, compounds may comprise or consist of one or more (additional) modifications to the nucleobase, scaffold and/or backbone linkage, which may or may not be present in the same monomer, for instance at the 3′ and/or 5′ position. A scaffold modification indicates the presence of a modified version of the ribosyl moiety as naturally occurring in RNA (i.e., the pentose moiety), such as bicyclic sugars, tetrahydropyrans, hexoses, morpholinos, 2′-modified sugars, 4′-modified sugar, 5′-modified sugars and 4′-substituted sugars. Examples of suitable modifications include, but are not limited to 2′-O-modified RNA monomers, such as 2′-O-alkyl or 2′-O-(substituted)alkyl such as 2′-OMe, 2′-O-(2-cyanoethyl), 2′-MOE, 2′-O-(2-thiomethyl)ethyl, 2′-0-butyryl, 2′-O-propargyl, 2′-O-allyl, 2′-O-(2-aminopropyl), 2′-O-(2-(dimethylamino)propyl), 2′-O-(2-amino)ethyl, 2′-O-(2-(dimethylamino)ethyl); 2′-deoxy (DNA); 2′-O-(haloalkyl)methyl such as 2′-0-(2-chloroethoxy)methyl (MCEM), 2′-O-(2,2-dichloroethoxy)methyl (DCEM); 2′-O-alkoxycarbonyl such as 2′-O-[2-(methoxycarbonyl)ethyl](MOCE), 2′-O-[2-N-methylcarbamoyl)ethyl](MCE), 2′-O-[2-(N,N-dimethylcarbamoyl)ethyl](DCME); 2′-halo e.g. 2′-F, FANA; 2′-O-[2-(methylamino)-2-oxoethyl](NMA); a bicyclic or bridged nucleic acid (BNA) scaffold modification such as a conformationally restricted nucleotide (CRN) monomer, a locked nucleic acid (LNA) monomer, a xylo-LNA monomer, an α-LNA monomer, an α-I-LNA monomer, a β-d-LNA monomer, a 2′-amino-LNA monomer, a 2′-(alkylamino)-LNA monomer, a 2′-(acylamino)-LNA monomer, a 2′-N-substituted 2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNANc(NH) monomer, a 2′,4′-BNANc(NMe) monomer, a 2′,4′-BNANC(NBn) monomer, an ethylene-bridged nucleic acid (ENA) monomer, a carba-LNA (cLNA) monomer, a 3,4-dihydro-2H-pyran nucleic acid (DpNA) monomer, a 2′-C-bridged bicyclic nucleotide (CBBN) monomer, an oxo-CBBN monomer, a heterocyclic-bridged BNA monomer (such as triazolyl or tetrazolyl-linked), an amido-bridged BNA monomer (such as AmNA), an urea-bridged BNA monomer, a sulfonamide-bridged BNA monomer, a bicyclic carbocyclic nucleotide monomer, a TriNA monomer, an α-I-TriNA monomer, a bicyclo DNA (bcDNA) monomer, an F-bcDNA monomer, a tricyclo DNA (tcDNA) monomer, an F-tcDNA monomer, an alpha anomeric bicyclo DNA (abcDNA) monomer, an oxetane nucleotide monomer, a locked PMO monomer derived from 2′-amino LNA, a guanidine-bridged nucleic acid (GuNA) monomer, a spirocyclopropylene-bridged nucleic acid (scpBNA) monomer, and derivatives thereof; cyclohexenyl nucleic acid (CeNA) monomer, altriol nucleic acid (ANA) monomer, hexitol nucleic acid (HNA) monomer, fluorinated HNA (F-HNA) monomer, pyranosyl-RNA (p-RNA) monomer, 3′-deoxypyranosyl DNA (p-DNA), unlocked nucleic acid UNA); an inverted version of any of the monomers above. All these modifications are known to the person skilled in the art.

The base sequence of the EON herein is complementary to part of the base sequence of a target HFE transcription product that includes at least a target adenosine (preferably the adenosine at position 845) that is to be deaminated to an inosine, and therefore can anneal (or hybridize) to the target transcription product. The complementarity of a base sequence can be determined by using a BLAST program or the like. Those skilled in the art can easily determine the conditions (temperature, salt concentration, and the like) under which two strands can be hybridized, taking into consideration the complementarity between the strands.

The EON herein, in contrast to what has been described for gapmers and their relation towards RNase breakdown and the use of such gapmers in double-stranded complexes (see for instance EP 3954395 A1), does not comprise a stretch of DNA nucleotides that would make a target sequence (or a sense nucleic acid strand) a target for RNase-mediated breakdown. In one embodiment, the EON does not comprise four or more consecutive DNA nucleotides anywhere within its sequence. In an embodiment, the EON is composed of as much (chemically) modified nucleotides as possible to enhance the resistance towards RNase-mediated breakdown, while at the same time being as efficient as possible in producing an RNA editing effect. This means that the orphan nucleotide and several other nucleotides within the EON may be DNA, but also that there is no stretch of four or more consecutive DNA nucleotides within the EON. Hence, the EON herein is not a gapmer. A gapmer reduces the expression of a target transcript but does not produce RNA editing of a specified adenosine within the target transcript. A gapmer is in principle a ss nucleic acid consisting of a central region (DNA gap region with at least four consecutive deoxyribonucleotides) and wing regions positioned directly at the 5′ end (5′ wing region) and the 3′ end (3′ wing region) thereof. In contrast, the EON herein may be any oligonucleotide that produces an RNA editing effect in which a target adenosine in a target RNA molecule is deaminated to an inosine, and accordingly is resistant to RNase-mediated breakdown as much as possible to yield this effect.

In one embodiment, the EON, or the sense strand to which it may be annealed before entering a target cell, is bound to a hydrophobic moiety, such as palmityl or an analog thereof, cholesterol or analog thereof, or tocopherol or analog thereof. It is preferably bound to the 5′ terminus. In case a hydrophobic moiety is bound to the 5′ terminus as well as to the 3′ terminus, such hydrophobic moieties may the same or different. The hydrophobic moiety bound to the oligonucleotide may be bound directly, or indirectly mediated by another substance. When the hydrophobic moiety is bound directly, it is sufficient if the moiety is bound via a covalent bond, an ionic bond, a hydrogen bond, or the like. When the hydrophobic moiety is bound indirectly, it may be bound via a linking group (a linker). The linker may be a cleavable or an uncleavable linker. A cleavable linker refers to a linker that can be cleaved under physiological conditions, for example, in a cell or an animal body (e.g., a human body). A cleavable linker is selectively cleaved by an endogenous enzyme such as a nuclease, or by physiological circumstances specific to parts of the body or cell, such as pH or reducing environment (such as glutathione concentrations). Examples of a cleavable linker comprise, but is not limited to, an amide, an ester, one or both esters of a phosphodiester, a phosphoester, a carbamate, and a disulfide bond, as well as a natural DNA linker. Cleavable linkers also include self-immolative linkers. An uncleavable linker refers to a linker that is not cleaved under physiological conditions, or very slowly compared to a cleavable linker, for example, in a PS linkage, modified or unmodified deoxyribonucleosides linked by a PS linkage, a spacer connected through a PS bond and a linker consisting of modified or unmodified ribonucleosides. There is no restriction on the chain length, when a linker is a nucleic acid such as DNA, or an oligonucleotide. However, it may be usually from 2 to 20 bases in length, from 3 to 10 bases in length, or from 4 to 6 bases in length. There is no restriction on the length or composition of a spacer that is connects the ligand and the oligonucleotide, and may include for example ethylene glycol, TEG, HEG, alkyl chains, propyl, 6-aminohexyl, or dodecyl.

The disclosure also provides a pharmaceutical composition comprising the EON disclosed herein, and further comprising a pharmaceutically acceptable carrier and/or other additive and may be dissolved in a pharmaceutically acceptable organic solvent, or the like. Dosage forms in which the EON or the pharmaceutical composition are administered may depend on the disorder to be treated and the tissue that needs to be targeted and can be selected according to common procedures in the art. The pharmaceutical compositions may be administered by a single-dose administration or by multiple dose administration. It may be administered daily or at appropriate time intervals, which may be determined using common general knowledge in the field and may be adjusted based on the disorder and the efficacy of the active ingredient.

In one embodiment, the EON comprises at least one nucleotide with a sugar moiety that comprises a 2′-OMe modification. In one embodiment, the EON comprises at least one nucleotide with a sugar moiety that comprises a 2′-MOE modification. In one embodiment, the EON comprises at least one nucleotide with a sugar moiety that comprises a 2′-F modification. In one embodiment, the orphan nucleotide carries a 2′-H in the sugar moiety and is therefore referred to as a DNA nucleotide, even though additional modifications may exist in its base and/or linkage to its neighbouring nucleosides. In one embodiment, the orphan nucleotide carries a 2′-F in the sugar moiety. In one embodiment, the orphan nucleotide carries a diF substitution in the sugar moiety. In one embodiment, the orphan nucleotide carries a 2′-F and a 2′-C-methyl in the sugar moiety. In one embodiment, the orphan nucleotide comprises a 2′-F in the arabinose configuration (FANA) in the sugar moiety. In one embodiment, the EON is an antisense oligonucleotide that can form a double stranded nucleic acid complex with a target RNA molecule, wherein the double stranded nucleic acid complex can recruit an adenosine deaminating enzyme for deamination of a target adenosine in the target HFE RNA molecule, wherein the nucleotide in the EON that is opposite the target adenosine is the orphan nucleotide, and wherein the orphan nucleotide has the following structure:

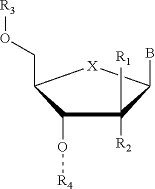

wherein: X is O, NH, OCH₂, CH₂, Se, or S; B is a nitrogenous base selected from the group consisting of: cytosine, uracil, isouracil, N3-glycosylated uracil, pseudoisocytosine, 8-oxo-adenine, and 6-amino-5-nitro-3-yl-2(1H)-pyridone; R₁ and R₂ are both selected, independently, from H, OH, F or CH₃; R₃ is the part of the EON that is 5′ of the orphan nucleotide, consisting of 7 to 30 nucleotides; and R₄ is the part of the EON that is 3′ of the orphan nucleotide, consisting of 4 to 25 nucleotides. The nucleotide 3′ and/or 5′ from the orphan nucleotide may be DNA, more preferably the nucleotide at the 3′ (position −1).

In one embodiment, the first nucleic acid strand comprises at least one methylphosphonate (MP) internucleoside linkage according to the following structure:

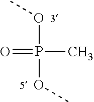

A preferred position for an MP linkage in an EON herein is linkage position −2, thereby connecting the nucleoside at position −1 with the nucleoside at position −2, although other positions for MP linkages are not explicitly excluded.

In one embodiment, the EON comprises at least one nucleotide with a sugar moiety that comprises a 2′-fluoro (2′-F) modification. A preferred position for the nucleotide that carries a 2′-F modification is position −3 in EON, which may be present together with an identical 2′ modification in the orphan nucleotide as discussed above.

In one embodiment, the EON comprises at least one phosphonoacetate or phosphonoacetamide internucleoside linkage.

In one embodiment, the EON comprises at least one nucleotide comprising a locked nucleic acid (LNA) ribose modification, or an unlocked nucleic acid (UNA) ribose modification. In an embodiment, the EON comprises at least one nucleotide comprising a TNA ribose modification.

The skilled person knows that an oligonucleotide, such as an EON as outlined herein, generally consists of repeating monomers. Such a monomer is most often a nucleotide or a chemically modified nucleotide. The most common naturally occurring nucleotides in RNA are adenosine monophosphate (A), cytidine monophosphate (C), guanosine monophosphate (G), and uridine monophosphate (U). These consist of a pentose sugar, a ribose, a 5′-linked phosphate group which is linked via a phosphate ester, and a 1′-linked base. The sugar connects the base and the phosphate and is therefore often referred to as the “scaffold” of the nucleotide.

A modification in the pentose sugar is therefore often referred to as a ‘scaffold modification’. The original pentose sugar may be replaced in its entirety by another moiety that similarly connects the base and the phosphate. It is therefore understood that while a pentose sugar is often a scaffold, a scaffold is not necessarily a pentose sugar. Examples of scaffold modifications that may be applied in the monomers of the EON of the present invention are disclosed in WO2020/154342, WO2020/154343, and WO2020/154344.

In one embodiment, the EON herein may comprise one or more nucleotides carrying a 2′-MOE ribose modification. Also, in one embodiment, the EON comprises one or more nucleotides not carrying a 2′-MOE ribose modification, and wherein the 2′-MOE ribose modifications are at positions that do not prevent the enzyme with adenosine deaminase activity from deaminating the target adenosine. In another embodiment, the EON comprises 2′-OMe ribose modifications at the positions that do not comprise a 2′-MOE ribose modification, and/or wherein the oligonucleotide comprises deoxynucleotides at positions that do not comprise a 2′-MOE ribose modification. In one embodiment the EON comprises one or more nucleotides comprising a 2′ position comprising a 2′-MOE, 2′-OMe, 2′-OH, 2′-deoxy, TNA, 2′-fluoro (2′-F), 2′,2′-difluoro (diF) modification, 2′-fluoro-2′-C-methyl modification, or a 2′-4′-linkage (i.e., a bridged nucleic acid such as a locked nucleic acid (LNA or examples mentioned in e.g. WO2018/007475)). In another embodiment, other nucleic acid monomer that are applied are arabinonucleic acids and 2′-deoxy-2′-fluoroarabinonucleic acid (FANA), for instance for improved affinity purposes. The 2′-4′ linkage can be selected from linkers known in the art, such as a methylene linker or constrained ethyl linker. A wide variety of 2′ modifications are known in the art. Further examples are disclosed in further detail in WO2016/097212, WO2017/220751, WO2018/041973, WO2018/134301, WO2019/219581, WO2019/158475, and WO2022/099159 for instance. In all cases, the modifications should be compatible with editing such that the EON fulfils its role as an editing producing oligonucleotide that can form a double stranded complex with the target RNA and recruit a deaminating enzyme, that can subsequently deaminate the target adenosine. Where a monomer comprises an unlocked nucleic acid (UNA) ribose modification, that monomer can have a 2′ position comprising the same modifications discussed above, such as a 2′-MOE, a 2′-OMe, a 2′-OH, a 2′-deoxy, a 2′-F, a 2′,2′-diF, a 2′-fluoro-2′-C-methyl, an arabinonucleic acid, a FANA, or a 2′-4′-linkage (i.e., a bridged nucleic acids such as a locked nucleic acid (LNA)).

A base, sometimes called a nucleobase, is generally adenine, cytosine, guanine, thymine or uracil, or a derivative thereof. A base, sometimes called a nucleobase, is defined as a moiety that can bond to another nucleobase through H-bonds, polarized bonds (such as through CF moieties) or aromatic electronic interactions. Cytosine, thymine, and uracil are pyrimidine bases, and are generally linked to the scaffold through their 1-nitrogen. Adenine and guanine are purine bases and are generally linked to the scaffold through their 9-nitrogen. The terms ‘adenine’, ‘guanine’, ‘cytosine’, ‘thymine’, ‘uracil’ and ‘hypoxanthine’ as used herein refer to the nucleobases as such. The terms ‘adenosine’, ‘guanosine’, ‘cytidine’, ‘thymidine’, ‘uridine’ and ‘inosine’ refer to the nucleobases linked to the (deoxy)ribosyl sugar.

The nucleobases in an EON herein can be adenine, cytosine, guanine, thymine, or uracil or any other moiety able to interact with another nucleobase through H-bonds, polarized bonds (such as CF) or aromatic electronic interactions. The nucleobases at any position in the nucleic acid strand can be a modified form of adenine, cytosine, guanine, or uracil, such as hypoxanthine (the nucleobase in inosine), pseudouracil, pseudocytosine, isouracil, N3-glycosylated uracil, 1-methylpseudouracil, orotic acid, agmatidine, lysidine, 2-thiouracil, 2-thiothymine, 5-substituted pyrimidine (e.g., 5-halouracil, 5-halomethyluracil, 5-trifluoromethyluracil, 5-propynyluracil, 5-propynylcytosine, 5-aminomethyluracil, 5-hydroxymethyluracil, 5-formyluracil, 5-aminomethylcytosine, 5-formylcytosine), 5-hydroxymethylcytosine, 7-deazaguanine, 7-deazaadenine, 7-deaza-2,6-diaminopurine, 8-aza-7-deazaguanine, 8-aza-7-deazaadenine, 8-aza-7-deaza-2,6-diaminopurine, 8-oxo-adenine, 3-deazapurine (such as a 3-deaza-adenosine), pseudoisocytosine, N4-ethylcytosine, N2-cyclopentylguanine, N2-cyclopentyl-2-aminopurine, N2-propyl-2-aminopurine, 2,6-diaminopurine, 2-aminopurine, G-clamp and its derivatives, Super A, Super T, Super G, amino-modified nucleobases or derivatives thereof; and degenerate or universal bases, like 2,6-difluorotoluene, or absent like abasic sites (e.g. 1-deoxyribose, 1,2-dideoxyribose, 1-deoxy-2-O-methylribose, azaribose).

In an embodiment, the nucleotide analog is an analog of a nucleic acid nucleotide. In an embodiment, the nucleotide analog is an analog of adenosine, guanosine, cytidine, thymidine, uridine, deoxyadenosine, deoxyguanosine, deoxycytidine, deoxythymidine or deoxyuridine. In an embodiment, the nucleotide analog is not guanosine or deoxyguanosine. In an embodiment, the nucleotide analog is not a nucleic acid nucleotide. In an embodiment, the nucleotide analog is not adenosine, guanosine, cytidine, thymidine, uridine, deoxyadenosine, deoxyguanosine, deoxycytidine, deoxythymidine, or deoxyuridine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted on this copolymer, namely to the scaffold moieties. Because of this characteristic, the alternating copolymer formed by linked scaffolds of an oligonucleotide is often called the ‘backbone’ of the oligonucleotide. Because phosphodiester bonds connect neighboring monomers together, they are often referred to as ‘backbone linkages’. It is understood that when a phosphate group is modified so that it is instead an analogous moiety such as a PS, such a moiety is still referred to as the backbone linkage of the monomer. This is referred to as a ‘backbone linkage modification’. In general terms, the backbone of an oligonucleotide comprises alternating scaffolds and backbone linkages.

EONs herein can comprise linkage modifications. A linkage modification can be, but not limited to, a modified version of the phosphodiester present in RNA, such as PS, chirally pure PS, (R)-PS, (S)-PS, MP, chirally pure methyl phosphonate, (R)-methyl phosphonate, (S)-methyl phosphonate, phosphoryl guanidine (such as PNdmi), chirally pure phosphoryl guanidine, (R)-phosphoryl guanidine, (S)-phosphoryl guanidine, phosphorodithioate (PS2), phosphonacetate (PACE), phosphonoacetamide (PACA), thiophosphonoacetate, thiophosphonoacetamide, methyl phosphorohioate, methyl thiophosphonate, PS prodrug, alkylated PS, H-phosphonate, ethyl phosphate, ethyl PS, boranophosphate, borano PS, methyl boranophosphate, methyl borano PS, methyl boranophosphonate, methyl boranophosphothioate, phosphate, phosphotriester, aminoalkylphosphotriester, and their derivatives. Another modification includes phosphoramidite, phosphoramidate, N3′→P5′ phosphoramidate, phosphorodiamidate, phosphorothiodiamidate, sulfamate, diethylenesulfoxide, amide, sulfonate, siloxane, sulfide, sulfone, formacetyl, alkenyl, methylenehydrazino, sulfonamide, triazole, oxalyl, carbamate, methyleneimino (MMI), and thioacetamide nucleic acid (TANA); and their derivatives. Various salts, mixed salts and free acid forms are also included, as well as 3′→3′ and 2′→5′ linkages.

In one embodiment, an EON comprises a substitution of one of the non-bridging oxygens in the phosphodiester linkage. This modification slightly destabilizes base-pairing but adds significant resistance to nuclease degradation. A preferred nucleotide analogue or equivalent comprises PS, phosphonoacetate, phosphorodithioate, phosphotriester, aminoalkylphosphotriester, H-phosphonate, methyl and other alkyl phosphonate including 3′-alkylene phosphonate, 5′-alkylene phosphonate and chiral phosphonate, phosphinate, phosphoramidate including 3′-amino phosphoramidate and aminoalkylphosphoramidate, thionophosphoramidate, thionoalkylphosphonate, thionoalkylphosphotriester, selenophosphate or boranophosphate. Particularly preferred are internucleoside linkages that are modified to contain a PS. Many of these non-naturally occurring modifications of the linkage, such as PS are chiral, which means that there are Rp and Sp configurations, known to the person skilled in the art. In one embodiment, the chirality of the PS linkages is controlled, which means that each of the linkages is either in the Rp or in the Sp configuration, whichever is preferred. The choice of an Rp or Sp configuration at a specified linkage position may depend on the target sequence and the efficiency of binding and induction of providing RNA editing. However, if such is not specifically desired, a composition may comprise AONs as active compounds with both Rp and Sp configurations at a certain specified linkage position. Mixtures of such EONs are also feasible, wherein certain positions have preferably either one of the configurations, while for other positions such does not matter.

Again, in all cases, the modifications should be compatible with editing such that the EON fulfils its role as an editing producing oligonucleotide that can, when attached to its target sequence recruit an adenosine deaminase enzyme because of the dsRNA nature that arises. In all aspects of the invention, the enzyme with adenosine deaminase activity is preferably ADAR1, ADAR2, or ADAT. In a highly preferred embodiment, the EON is an RNA editing oligonucleotide that targets a pre-mRNA or an mRNA, wherein the target nucleotide is an adenosine in the target RNA, wherein the adenosine is deaminated to an inosine, which is being read as a guanosine by the translation machinery. The disclosure also provides a pharmaceutical composition comprising the EON as characterized herein, and a pharmaceutically acceptable carrier.

Other chemical modifications of the EON herein include the substitution of one or more than one of any of the hydrogen atoms with deuterium or tritium, examples of which can be found in e.g., WO2014/022566 or WO2015/011694.

The disclosure provides an EON herein, or a pharmaceutical composition comprising an EON herein, for use in the treatment or prevention of a disorder related to iron overload. In one embodiment, the disclosure provides an EON herein, or a pharmaceutical composition comprising an EON herein, for use in the treatment or prevention of a disease related to iron overload, such as HH. In one embodiment, the provides an EON herein, or a pharmaceutical composition comprising an EON herein, for use in the treatment or prevention of HH.

EONs herein preferably do not include a 5′-terminal O6-benzylguanosine or a 5′-terminal amino modification and preferably are not covalently linked to a SNAP-tag domain (an engineered O6-alkylguanosine-DNA-alkyl transferase). EONs herein preferably do not comprise a boxB RNA hairpin sequence. In one embodiment, an EON herein comprises 0, 1, 2 or 3 wobble base pairs with the target sequence, and/or 0, 1, 2, 3, 4, 5, 6, 7, or 8 mismatching base pairs with the target RNA sequence. No mismatch exists when the orphan nucleotide is uridine. One alternative for uridine is positioning an iso-uridine opposite the target adenosine, which likely does not pair like G pairs with U. Preferably, the target adenosine in the target sequence forms a mismatch base pair with the nucleoside in the EON that is directly opposite the target adenosine.

It should be noted that when an EON is delivered through a vector, for instance an AAV vector, chemical modifications are not present in the EON that acts on the target RNA molecule. Although it is preferred to use ‘naked’ EONs that have chemical modifications as outlined herein, EONs that are delivered through other means, for instance through AAV vector expression, or editing molecules that are circular, or have hairpin structures (recruiting portions, e.g., as disclosed in WO2016/097212, WO2017/050306, WO2020/001793, WO2017/010556, WO2020/246560, and WO2022/078995) are also encompassed by the present invention because these can also be applied to edit adenosines in the target HFE RNA molecule to generate a HFE protein with restored function.

An EON herein can utilise endogenous cellular pathways and naturally available ADAR enzymes to specifically edit a target adenosine in the target RNA sequence. An EON herein is capable of recruiting ADAR and complex with it and then facilitates the deamination of a (single) specific target adenosine nucleotide in a target RNA sequence. Ideally, only one adenosine is deaminated. An EON herein, when complexed to ADAR, preferably brings about the deamination of a single target adenosine.

Analysis of natural targets of ADAR enzymes has indicated that these generally include mismatches between the two strands that form the RNA helix edited by ADAR1 or 2. It has been suggested that these mismatches enhance the specificity of the editing reaction (Stefl et al. 2006. Structure 14(2):345-355; Tian et al. 2011. Nucleic Acids Res 39(13):5669-5681). Characterization of optimal patterns of paired/mismatched nucleotides between the EONs and the target RNA also appears important to the development of efficient ADAR-based EON therapy.

As outlined above, an EON herein makes use of specific nucleotide modifications at predefined spots to ensure stability as well as proper ADAR binding and activity. These changes may vary and may include modifications in the backbone of the EON, in the sugar moiety of the nucleotides as well as in the nucleobases or the phosphodiester linkages, as outlined in detail herein. They may also be variably distributed throughout the sequence of the EON. Specific modifications may be needed to support interactions of different amino acid residues within the RNA-binding domains of ADAR enzymes, as well as those in the deaminase domain. For example, PS linkages between nucleotides or 2′-OMe or 2′-MOE modifications may be tolerated in some parts of the EON, while in other parts they should be avoided so as not to disrupt crucial interactions of the enzyme with the phosphate and 2′—OH groups. Specific nucleotide modifications may also be necessary to enhance the editing activity on substrate RNAs where the target sequence is not optimal for ADAR editing. Previous work has established that certain sequence contexts are more amenable to editing. For example, a target sequence 5′-UAG-3′ (with the target A in the middle) contains the most preferred nearest-neighbor nucleotides for ADAR2, whereas a 5′-CAA-3′ target sequence is disfavored (Schneider et al. 2014. Nucleic Acids Res 42(10):e87). The structural analysis of ADAR2 deaminase domain hints at the possibility of enhancing editing by careful selection of the nucleotides that are opposite to the target trinucleotide. For example, the 5′-CAA-3′ target sequence, paired to a 3′-GCU-5′ sequence on the opposing strand (with the A-C mismatch formed in the middle), is disfavored because the guanosine base sterically clashes with an amino acid side chain of ADAR2. Although other adenosines in the HFE transcript may be targeted to impair the protein function, in a preferred aspect, the adenosine at position 845 is deaminated. The disclosure provides RNA editing oligonucleotides, generally referred to as EONs herein, that can bring about deamination of an adenosine in the HFE transcript, with a resulting HFE protein that is fully functional in iron level control. This means that the invention is not strictly limited to deamination of the adenosine at position 845, but that other (single or multiple) adenosines may be targeted, which may also result in increased HFE protein function. Other adenosines may be identified, for instance by genetic screening in the population, or in silico, that are also important (or may become more important) for HFE function, and that also may be targeted through RNA editing, following the teaching of the present disclosure. All such RNA events and oligonucleotides that can be used for such targeting are encompassed by the disclosure, no matter what the exact nucleic molecule, or EON, looks like.

Mutagenesis studies of human ADAR2 revealed that a single mutation at residue 488 from glutamate to glutamine (E488Q), gave an increase in the rate constant of deamination by 60-fold when compared to the wild-type enzyme (Kuttan and Bass. 2012. Proc Natl Acad Sci USA. 109(48):3295-3304). During the deamination reaction, ADAR flips the edited base out of its RNA duplex, and into the enzyme active site (Matthews et al. 2016. Nat Struct Mol Biol. 23(5):426-433). When ADAR2 edits adenosines in the preferred context (an A:C mismatch) the nucleotide opposite the target adenosine is often referred to as the ‘orphan cytidine’. The crystal structure of ADAR2 E488Q bound to double stranded RNA (dsRNA) revealed that the glutamine (Gln) side chain at position 488 can donate an H-bond to the N3 position of the orphan cytidine, which leads to the increased catalytic rate of ADAR2 E488Q. In the wild-type enzyme, wherein a glutamate (Glu) is present at position 488 instead of a glutamine (Gln) the amide group of the glutamine is absent and is instead a carboxylic acid. To obtain the same contact of the orphan cytidine with the E488Q mutant would then, for the wild-type situation, require protonation for this contact to occur. To make use of endogenously expressed ADAR2 to correct disease relevant mutations, it is essential to maximize the editing efficiency of the wild type ADAR2 enzyme present in the cell. WO2020/252376 discloses the use of EONs with modified RNA bases, especially at the position of the orphan cytidine to mimic the hydrogen-bonding pattern observed by the E488Q ADAR2 mutant. By replacing the nucleotide opposite the target adenosine in the EON with cytidine analogs that serve as H-bond donors at N3, it was envisioned that it would be possible to stabilize the same contact that is believed to provide the increase in catalytic rate for the mutant enzyme. Two cytidine analogs were of particular interest: pseudoisocytidine (also referred to as ‘piC’; Lu et al. 2009. J Org Chem. 74(21):8021-8030; Burchenal et al. 1976. Cancer Res 36:1520-1523) and Benner's base Z (also referred to as ‘dZ’; Yang et al. 2006. Nucl Acid Res. 34(21):6095-6101) that were initially selected because they offer hydrogen-bond donation at N3 with minimal perturbation to the shape of the nucleobase. Benner's base is also referred to as 6-amino-5-nitro-3-yl-2(1H)-pyridone. The presence of the cytidine analog in the AON may exist in addition to modifications to the ribose 2′ group. The ribose 2′ groups in the AON can be independently selected from 2′-H (i.e., DNA), 2′-OH (i.e., RNA), 2′-OMe, 2′-MOE, 2′-F, or 2′-4′-linked (i.e., a bridged nucleic acid such as a locked nucleic acid (LNA)), or other 2′ substitutions. The 2′-4′ linkage can be selected from linkers known in the art, such as a methylene linker or constrained ethyl linker.

In one embodiment, an EON herein comprises one or more sugar moieties that are mono- or di-substituted at the 2′, 3′ and/or 5′ position such as: —OH; —H; —F; substituted or unsubstituted, linear or branched lower (C₁-C₁₀) alkyl, alkenyl, alkynyl, alkaryl, allyl, or aralkyl, that may be interrupted by one or more heteroatoms; —O-, S-, or N-alkyl; —O-, S-, or N-alkenyl; —O-, S-, or N-alkynyl; —O-, S-, or N-allyl; —O-alkyl-O-alkyl; -methoxy; -aminopropoxy; -methoxyethoxy; -dimethylamino oxyethoxy; and -dimethylaminoethoxyethoxy.

In one embodiment, a nucleotide analogue or equivalent within the EON herein comprises one or more base modifications or substitutions. Modified bases comprise synthetic and natural bases such as inosine, xanthine, hypoxanthine and other -aza, deaza, -hydroxy, -halo, -thio, thiol, -alkyl, -alkenyl, -alkynyl, thioalkyl derivatives of pyrimidine and purine bases that are or will be known in the art. Purine nucleobases and/or pyrimidine nucleobases may be modified to alter their properties, for example by amination or deamination of the heterocyclic rings. The exact chemistries and formats may vary from oligonucleotide construct to oligonucleotide construct and from application to application, and may be worked out in accordance with the wishes and preferences of those of skill in the art.

An EON herein is normally longer than 10 nucleotides, preferably more than 11, 12, 13, 14, 15, 16, still more preferably more than 17 nucleotides. In one aspect the EON herein is longer than 20 nucleotides. The EON herein is preferably shorter than 100 nucleotides, still more preferably shorter than 60 nucleotides, still more preferably shorter than 50 nucleotides. In a preferred aspect, the EON herein comprises 18 to 70 nucleotides, more preferably comprises 18 to 60 nucleotides, and even more preferably comprises 18 to 50 nucleotides. Hence, in a particularly preferred aspect, the EON herein comprises 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. In one embodiment, the EON herein is 27, 28, 29, or 30 nucleotides in length.

In one aspect, at either end or both termini of an EON herein inverted deoxyT or dideoxyT nucleotides are incorporated.

As described above, in some embodiments the disclosure provides an EON for forming a ds complex with a human HFE RNA molecule in a human liver cell. Thus, the therapeutic effect is preferably on a human liver cell in vivo. Of course, the methods may also be carried out in vitro or ex vivo.

The disclosure provides an EON, or pharmaceutical composition, for use in the treatment of disease. The disclosure also provides the use of an EON herein, or pharmaceutical composition herein, in the manufacture of a medicament for the treatment of disease. The disclosure also provides a method for treating a disease in a patient, comprising administering a therapeutically effective amount of an EON herein or a pharmaceutical composition herein. Preferably the disease is a disease caused by iron overload caused by the C282Y mutation in HFE. The EON is preferably administered therapeutically, rather than prophylactically (after genetic counselling), but such cannot be excluded to be also beneficial.

After RNA editing has occurred in a cell, the modified RNA can become diluted over time, for example due to cell division, limited half-life of the edited RNAs, etc. Thus, in practical therapeutic terms a method of the invention may involve repeated delivery of an AON until enough target RNAs have been modified to provide a tangible benefit to the patient and/or to maintain the benefits over time.

### EXAMPLES

### Example 1. Editing of a Target Adenosine in a Human HFE Target RNA Molecule Using an In Vitro Biochemical Editing Assay

First, an initial set of the HFE-targeting EONs (RM4700 to RM4726; shown in FIG. 1) were tested to address editing of human HFE target (pre-) mRNA in an in vitro biochemical editing assay. To obtain the HFE target RNA, a PCR was performed using a HFE G-block (IDT) that contains the sequence for the T7 promotor and (a part of) the sequence of HFE as template using forward primer 5′-CTC GAC GCA AGC CAT AAC AC-3′ (SEQ ID NO:53) and reverse primer 5′-TGG ACC GAC TGG AAA CGT AG-3′ (SEQ ID NO:54). The 5′ to 3′ G-block sequence (SEQ ID NO:55) is as follows, in which the target adenosine is underlined and in bold, and in which the primer sequences are underlined:

TCTGGCTCGACGCAAGCCATAACACTAATACGACTCACTATAGGGAGTTCGAACCTAAAGACGTATTGCCCAATGGG
GATGGGACCTACCAGGGCTGGATAACCTTGGCTGTACCCCCTGGGGAAGAGCAGAGATATACGTACCAGGTGGAGCA
CCCAGGCCTGGATCAGCCCCTCATTGTGATCTGGGAGCCCTCACCGTCTGGCACCCTAGTCATTGGAGTCATCAGTG
GAATTGCTGTTTTTGTCGTACGTTTCCAGTCGGTCCACGTT

The PCR product was then used as template for the in vitro transcription. The MEGAscript T7 transcription kit was used for this reaction. The RNA was purified on a urea gel and then extracted in 50 mM Tris-Cl pH 7.4, 10 mM EDTA, 0.1% SDS, 0.3 M NaCl buffer and subsequently phenol-chloroform purified. The purified RNA was used as target in the biochemical editing assay.

Initially, EONs RM4700 to RM4726 were annealed to the HFE target RNA, which was done in a buffer (5 mM Tris-Cl pH 7.4, 0.5 mM EDTA and 10 mM NaCl) at the ratio 1:3 of target RNA to oligonucleotide (600 nM oligonucleotide and 200 nM target). The samples were heated at 95° C. for 3 min and then slowly cooled down to RT. Next, the editing reaction was carried out. The annealed oligonucleotide/target RNA was mixed with protease inhibitor (cOmplete™, Mini, EDTA-free Protease I, Sigma-Aldrich), RNase inhibitor (RNasin, Promega), poly A (Qiagen), tRNA (Invitrogen) and editing reaction buffer (15 mM Tris-Cl pH 7.4, 1.5 mM EDTA, 3% glycerol, 60 mM KCl, 0.003% NP-40, 3 mM MgCl₂ and 0.5 mM DTT) such that their final concentration was 6 nM oligonucleotide and 2 nM target RNA. The reaction was started by adding purified ADAR2 (GenScript) to a final concentration of 6 nM into the mix and incubated for predetermined time points at 37° C. Each reaction was stopped by adding 95 μl of 95° C. 3 mM EDTA solution. A 6 μl aliquot of the stopped reaction mixture was then used as template for cDNA synthesis using Maxima reverse transcriptase kit (Thermo Fisher) with random hexamer primer (ThermoFisher Scientific). Initial denaturation of RNA was performed in the presence of the primer and dNTPs at 95° C. for 5 min, followed by slow cooling to 10° C., after which first strand synthesis was carried out according to the manufacturer's instructions in a total volume of 20 μl, using an extension temperature of 62° C. Products were amplified for pyrosequencing analysis by PCR, using the Amplitaq gold 360 DNA Polymerase kit (Applied Biosystems) according to the manufacturer's instructions, with 1 μl of the cDNA as template. Then PCR was performed using the following thermal cycling protocol: Initial denaturation at 95° C. for 5 min, followed by 40 cycles of 95° C. for 30 sec, 58° C. for 30 sec and 72° C. for 30 sec, and a final extension of 72° C. for 7 min.

Because inosines base-pair with cytidines during the cDNA synthesis in the reverse transcription reaction, the nucleotides incorporated in the edited positions during PCR will be guanosines. The percentage of guanosine (edited) versus adenosine (unedited) was defined by pyrosequencing. Pyrosequencing of the PCR products and data analysis was performed by the PyroMark Q48 Autoprep instrument (QIAGEN) following the manufacturer's instructions with 10 μl input of the PCR product and 4 μM sequencing primer: The analysis performed by the instrument provides the results for the selected nucleotide as a percentage of adenosine and guanosine detected in that position, and the extent of A-to-I editing at a chosen position was therefore measured by the percentage of guanosine in that position.

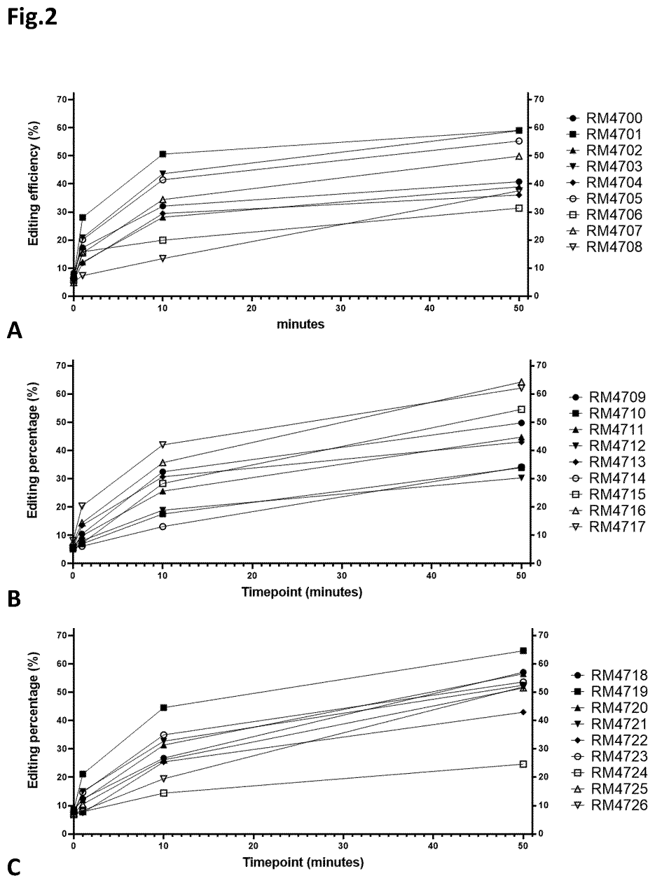

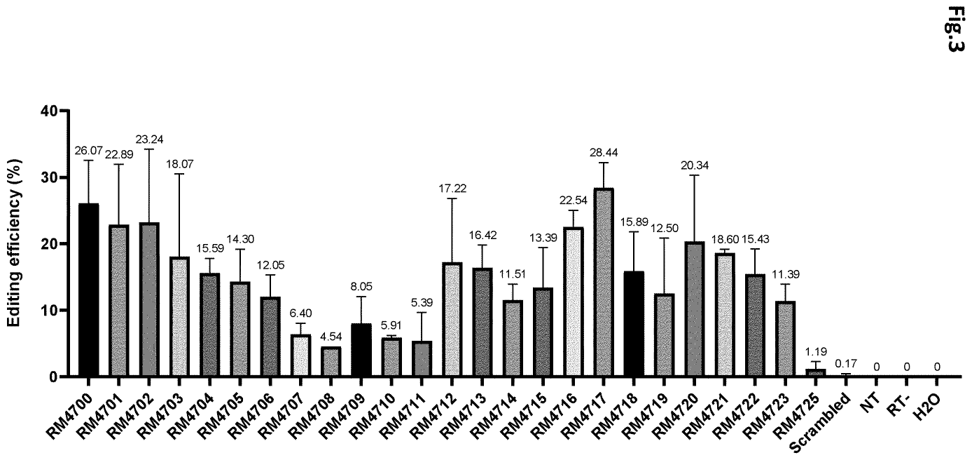

Results are given in FIGS. 2A, B and C each with a subset of data points from the given EONs. It can be clearly seen that all tested EONs were capable of mediating RNA editing in the biochemical editing assay, albeit with a variety of efficiencies. The best performing EONs in this in vitro assay were the EONs having 18 nucleotides on the 5′ side of the orphan nucleotide and 11 nucleotides on the 3′ side of the orphan nucleotide (exemplified by RM4716, RM4717, RM4718, and RM4719).

### Example 2. Editing of a Target Adenosine in a Human HFE Target RNA Molecule in B-Lymphocytes from Donors Carrying the C282Y Mutation

Next, EONs RM4700 to RM4723 and RM4725 were tested for their ability to mediate RNA editing by recruitment of endogenously present ADAR enzymes in B-lymphocytes from two different donors being homozygous for the C282Y (c.845G>A) mutation in the HFE gene. These donors are referred to as GM14715 and GM14631 and the B-lymphocytes were provided by Corriel. The human Epstein-Barr virus (EBV) immortalized B-lymphocyte cells were cultured in RPMI-1640/10% FBS/1% Pen-Strep. Cells were kept at 37° C. in a 5% CO₂ atmosphere.

A total of 0.2×10⁶ cells were co-treated with 5 μM EON+1 μM AG1856 saponin in a total of 200 μl volume in a 48 well format. The skilled person is aware that a variety of different saponins have been used for many types of applications, and that therapeutic uses of saponins have also been described (Weng A et al. 2009. Planta medica 75(13):1421-1422; Weng A et al. 2010. J Chromatography B 878(7):713-718; Weng A et al. 2012. Molecular Oncology 6(3):323-332; Weng A et al. 2012. J Controlled Disease 164(1):74-86; Thakur et al. 2014. J Chromatography B 955:1-9; Jia et al. 1998. J Natural Products 61(11):1368-1373; Haddad et al. 2004. Helvetica chimica acta 87(1):73-81; Fu et al. 2005. J Natural Products 68(5):754-758; Moniuszko-Szajwaj et al. 2016. Helvetica chimica acta 99(5):347-354; Fuchs H et al. 2017. Biomedicines 5(2):14). It has also been described that a specific saponin (SO1861) derived from Saponaria officinalis can mediate an improved intracellular delivery of peptide and lipid nanoparticles, as well as nucleic acids (Weng A et al. 2015. J Controlled Release 206:75-90; Sama S et al. 2017. Int J Pharmaceutics 534:195-205). WO2019/011914 discloses a saponin (GE1741) isolated from Gypsophila elegans rendering improved effects regarding the delivery of small molecules, such as nucleic acid molecules to cells (see also Sama S et al. 2018. J Biotechnology 284:131-139). WO2021/122998 (and EP3838910B1, accruing from the priority application) discloses yet another class of saponins, derived from Agrostemma githago L. with further improved properties over the earlier described SO1861 and GE1741 saponins, especially regarding toxicity and endosomal escape (see also Clochard J et al. 2020. Int J Pharm 589:119822). The inventors of the present invention used the saponin AG1856 (also referred to as triterpene glycosides, or triterpene saponins) disclosed in WO2021/122998 for enhancing the effect on RNA editing of the HFE transcript in the B-lymphocytes discussed above.

Negative controls were a treated sample using an EON with a scrambled sequence (sequence not shown), a non-treated sample (NT), a sample where no reverse transcriptase (−RT) was used (see below) and a water sample.

72 hrs post initial exposure to the EONs and AG1856, cells were collected, and total RNA was isolated using the SV Total RNA Isolation System kit (Promega). After removal of the culture medium, cells were washed once with PBS. After complete aspiration of the PBS, 100 μL BL+TG (Promega) was added to lyse the cells and collect the intracellular material. After addition of 35 μL 2-propanol, the mixtures were loaded on a column and subjected to several wash steps and DNaseI treatment. After elution in a total volume of 20 μL DNase/RNase-free water, the RNA yield was determined using spectrophotometric analysis (NanoDrop) and stored at −80° C.

Maxima reverse transcriptase (RT, Thermo Fisher) was used to generate cDNA. Typically, 500 ng total RNA was used in reaction mixture containing 4 μL 5×RT buffer, 1 μL dNTP mix (10 mM each), 1 μL random hexamer (all Thermo Fisher) supplemented with DNase and RNase free water to a total volume of 20 μL. Samples were loaded in a T100 thermocycler (Bio-Rad) and initially incubated at 10 min at 25° C., followed by a cDNA reaction temperature of 30 min at 50° C. and a termination step of 5 min at 85° C. Samples were cooled down to 4° C. prior storing at −20° C.

To determine the editing efficiency, cDNA samples were used in digital PCR (dPCR) assays. The first dPCR is designed to distinguish between cDNA species containing the original adenosine or the edited inosine, which is converted into a guanidine during cDNA synthesis. The second multiplex dPCR quantifies total HFE transcript copies (cDNA molecules) in the mixture using a primer/probe set targeting exons 1 and 2. A third assay quantifies exon 5 skip using primers binding exon 4/7 and a probe overlapping the exon 4/6 boundary. The primer and probe sequences were as follows, wherein the “+” refers to an LNA nucleotide at the 3′ side:

| | | |
|---|---|---|
| Total assay | | |
| hHFE_e01_Fw | GGCGCTTCTCCTCCTGATG | SEQ ID NO: 56 |
| hHFE_e01-02_TEX | TGCTGCGTTCACACTCTCTGCAC | SEQ ID NO: 57 |
| hHFE_e02_rv | CCACGTAGCCCAAAGCTTCA | SEQ ID NO: 58 |
| Edit assay | | |
| hHFE_e04_fw | CGTATTGCCCAATGGGGATG | SEQ ID NO: 59 |
| hHFE_e04_edit_FAM | CAGAGATATACGT+G+CCAGGTGGA | SEQ ID NO: 60 |
| hHFE_e04_ori_HEX | CAGAGATATACG+T+A+CCAGGTGGA | SEQ ID NO: 61 |
| hHFE_e05_rv | ACTCCAATGACTAGGGTGCC | SEQ ID NO: 62 |
| Skip assay | | |
| hHFE_e04_fw | TGGGAAAGGCACAAGATTCG | SEQ ID NO: 63 |
| hHFE_e04-06_Cy5 | GGACCAACAAG+AGCCCTCAC | SEQ ID NO: 64 |
| hHFE_e07_rv | CAGCTAAGACGTAGTGCCCC | SEQ ID NO: 65 |

In total, 1.3 μL of the cDNA mix was used in a dPCR mixture containing 3 μL 4×dPCR mastermix (Qiagen), 0.6 μL of primers and 0.3 uL probe (10 μM stock concentration), supplemented with DNase and RNase free water to a total volume of 13 μL. Of this mixture 12 μL was transferred to an 8.5K partition plate and fluorophores measured on the Qiaquity apparatus. The dPCR cycling conditions were as follows: enzyme activation at 95° C. for 2 min, then 40 cycles of denaturation at 95° C. for 15 sec, annealing/extension at 63° C. for 30 sec. Percentage of A-to-I editing was determined by dividing the number of G-containing molecules by the total (G- plus A-containing species) multiplied by 100.

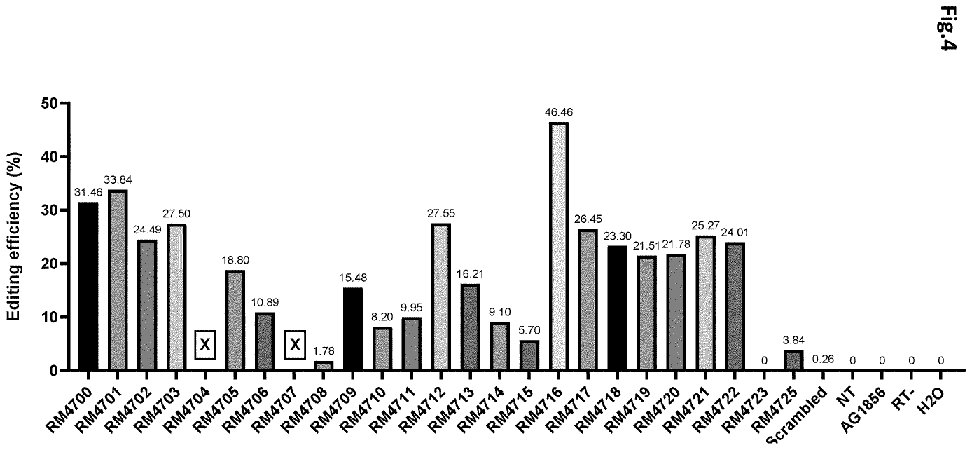

The results of the editing experiments using the B-lymphocytes from donor GM14715 are shown in FIG. 3, and clearly indicate that all tested EONs were able to mediate RNA editing of the mutation in the HFE transcript, albeit with different efficiencies. Up to almost 30% editing was observed with the best performing EON in this cell-based assay, which was EON RM4717. Exon 4 skipping in the HFE transcript was limited (around 2%) after treatment with the EONs (data not shown) and the amount of HFE transcript was relatively stable after EON treatment (data not shown). FIG. 4 shows the results of the editing experiments using the B-lymphocytes of donor GM14631, again showing a variety of efficiencies, but in line with the results shown in FIG. 3. RM4716 performed best reaching editing levels of almost 50%. The samples of RM4704 and RM4707 were lost during the measurements of this initial experiment (indicated by X). From these two experiments it became clear that EONs with 18 nucleotides 5′ of the orphan nucleotide and 9, 11 or 13 nucleotides 3′ of the orphan nucleotide provided the highest efficiencies. RM4725, which contains a long stretch of 2′-F at the 5′ end of the EON and a relatively short stretch of nucleotides at the 3′ end (only 5 nucleotides) gave relatively low RNA editing.

These experiments show that the inventors could achieve ADAR-mediated editing applying endogenous ADAR enzymes and RNA editing mediating oligonucleotides in EBV-immortalized B-lymphocytes from donors carrying two alleles of the HFE c.845G>A mutation.

### Example 3. Editing of a Target Adenosine in a Human HFE Target RNA Molecule in GM14715 B-Lymphocytes and Hepcidin Expression Upon Treatment

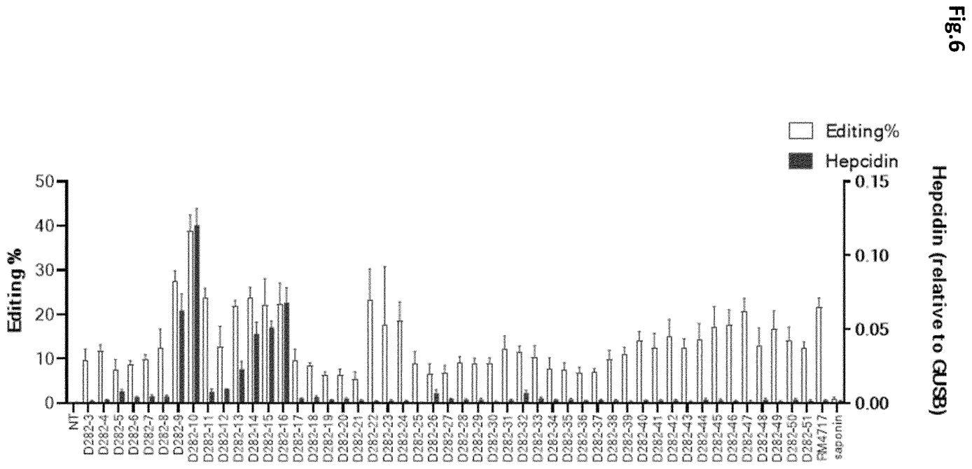

On top of the EONs designed initially (FIG. 1), a further set of EONs was designed with another variety of length and chemical modifications. These EONs with their modifications are shown in FIG. 5. Some of these were used in the treatment of GM14715 B-lymphocytes as outlined in Example 2, with a co-treatment of 2 μM saponin AG1856. The editing percentages were determined as described above, after 72 hrs treatment. FIG. 6 shows the editing percentages observed with these EONs, that were compared with RM4717 that performed well as shown in FIG. 3. The results in FIG. 6 clearly show that the best performer (D282-10; RM106443; SEQ ID NO:73) gave more than 40% editing of the target adenosine in the HFE transcript, which was even higher than initially observed with RM4717.

To determine the downstream functional effect of the RNA editing effect brought about by these EONs, it was investigated whether hepcidin expression levels were increased upon mRNA restoration of the C282Y mutation in B-lymphocytes. The same samples in which the editing percentages were determined were used to quantify the expression of HAMP. To do so, a dPCR assay was designed to detect HAMP mRNA in conjunction with the housekeeping gene GUSB which was used to normalize HAMP the expression data. The data presented in FIG. 6 shows that with high editing levels the expression of HAMP mRNA also increases.

| | | |
|---|---|---|
| HAMP assay | | |
| hHAMP_e01_fw | CCAGTGGCTCTGTTTTCC | SEQ ID NO: 165 |
| hHAMP_e01- | /5Cy5/AACAGACGG/TAO/GACAACTTGCA/ | SEQ ID NO: 166 |
| 02_Cy5 | 3IAbRQSp/ | |
| hHAMP_e03_rev | CTCTGGAACATGGGCATC | SEQ ID NO: 167 |
| GUSB assay | | |
| hGUSB_e04_fw | ATGACATCACCGTCACCACC | SEQ ID NO: 168 |
| hGUSB_e04- | TGGAGCAAG/ZEN/ACAGTGGGCTGGT | SEQ ID NO: 169 |
| 05_ATTO555 | | |
| hGUSB_e05_rv | CCATTCGCCACGACTTTGTT | SEQ ID NO: 170 |

### Example 4. Generating a C282Y Mutation Carrying Human Hepatocytes for In Vitro Screening of EONs

Since the liver plays a central role in the iron homeostasis it would be desired to test the EONS on a cell model that better reflects the target tissue. To do so, a human hepatocyte-like cell line is made. Since certain EONs carry a 3′-attached tri-antennary GalNAc moiety, these cells allow for GalNAc-assisted EON uptake via the hepatocyte expressed Asialoglycoprotein receptor (ASGR). A human induced pluripotent stem cell (iPSC) line is generated in which the C282Y (c.845G>A; rs1800562) is introduced in the HFE gene using a CRISPR/Cas9 gene editing approach. These iPSC C282Y cells are differentiated to mature hepatocyte-like cells in which the expression of mature hepatocyte markers and the ASGR is confirmed.

### Example 5. Quantifying Intracellular Iron Levels after Editing of a Target Adenosine in HFE Target RNA

Since one of the characteristic clinical manifestations of HH is high iron levels in serum and liver, an iron measurement assay is developed to quantify intracellular and tissue iron levels. The intent is to quantify iron levels after treatment with an HFE C282Y editing EON to determine the effect of HFE restoration on iron metabolism. Several iron quantification methods are explored, such as spectrophotometric methods where iron is complexed with a chromogen such as ferrozine or ferene-s allowing for subsequent colorimetric detection at a specific wavelength. Alternatively, inductively coupled plasma mass spectrometry (ICP-MS) is used to quantify the number of iron atoms within a cell lysate.

### Example 6. Quantifying Amino Acid Restoration in C282Y HFE after Editing of a Target Adenosine

To determine the impact of the EON mediated HFE restoration on its protein sequence, a method is developed which allows for the discrimination between, and quantification of, wild-type and mutant HFE (C282Y) protein. Liquid chromatography with tandem mass spectrometry (LC-MS/MS) is explored as a possible peptide quantification technique. This technique allows for the quantification of targeted HFE peptides in cells and tissues, ideally the assay is capable of detecting both the restored wild-type HFE peptide sequence encompassing the C282 amino acid (or the C294 HFE mouse equivalent), and the mutant HFE C282Y peptide.

1. An RNA editing oligonucleotide (EON) capable of forming a double-stranded complex with a region of an endogenous human HFE transcript molecule in a cell, wherein the region of the HFE transcript molecule comprises a target adenosine, and wherein the double-stranded complex can recruit an endogenous ADAR enzyme to deaminate the target adenosine into an inosine, thereby editing the HFE transcript molecule.

2. An EON according to claim 1, wherein the HFE transcript molecule is a pre-mRNA or an mRNA molecule.

3. An EON according to claim 1 or 2, wherein the cell is a human liver cell, preferably a hepatocyte.

4. An EON according to any one of claims 1 to 3, wherein the target adenosine is a c.845G>A mutation in the human HFE gene.

5. An EON according to any one of claims 1 to 4, wherein the EON comprises or consists of the nucleotide sequence of any one of the EON sequences of SEQ ID NO:1 to 51 and 66 to 164.

6. An EON according to any one of claims 1 to 5, wherein at least one nucleotide comprises one or more non-naturally occurring chemical modifications, or one or more additional non-naturally occurring chemical modifications, in the ribose, linkage, or base moiety, with the proviso that the orphan nucleotide, which is the nucleotide in the EON that is directly opposite the target adenosine, is not a cytidine comprising a 2′-OMe ribose substitution.

7. An EON according to claim 6, wherein the orphan nucleotide is a cytidine analog, preferably a deoxynucleotide comprising a 6-amino-5-nitro-3-yl-2(1H)-pyridone nucleobase.

8. An EON according to claim 6, wherein the orphan nucleotide is a uridine analog, preferably a deoxynucleotide comprising an iso-uracil nucleobase.

9. An EON according to any one of claims 6 to 8, wherein the one or more additional modifications in the linkage moiety is each independently selected from a phosphorothioate (PS), phosphonoacetate, phosphorodithioate, methylphosphonate (MP), sulfonylphosphoramidate, or PNdmi internucleotide linkage.

10. EON according to any one of claims 6 to 9, wherein the one or more additional modifications in the ribose moiety is a mono- or di-substitution at the 2′, 3′ and/or 5′ position of the ribose, each independently selected from the group consisting of:
—OH;
—F;
substituted or unsubstituted, linear or branched lower (C₁-C₁₀) alkyl, alkenyl, alkynyl, alkaryl, allyl, or aralkyl, that may be interrupted by one or more heteroatoms;
—O-, S-, or N-alkyl;
—O-, S-, or N-alkenyl;
—O-, S-, or N-alkynyl;
—O-, S-, or N-allyl;
—O-alkyl-O-alkyl;
-methoxy;
-aminopropoxy;
-methoxyethoxy;
-dimethylamino oxyethoxy; and
-dimethylaminoethoxyethoxy.

11. A vector, preferably a viral vector, more preferably an adeno-associated virus (AAV) vector, comprising a nucleic acid molecule encoding an EON according to any one of claims 1 to 5.

12. A Lipid Nanoparticle (LNP) formulation comprising an EON according to any one of claims 1 to 10.

13. A pharmaceutical composition comprising an EON according to any one of claims 1 to 10, a vector according to claim 11, or an LNP formulation according to claim 12; and a pharmaceutically acceptable carrier.

14. An EON according to claim 1 to 10, a vector according to claim 11, an LNP formulation according to claim 12, or a pharmaceutical composition according to claim 13, for use in the treatment of homeostatic iron regulator protein (HFE) hemochromatosis.

15. Use of an EON according to any one of claims 1 to 10 in the manufacture of a medicament for the treatment of homeostatic iron regulator protein (HFE) hemochromatosis.

16. A method of editing a HFE polynucleotide, the method comprising contacting the HFE polynucleotide with an EON according to any one of claims 1 to 10, thereby editing the HFE polynucleotide.

17. A method of treating homeostatic iron regulator protein (HFE) hemochromatosis in a patient in need thereof, the method comprising contacting a HFE polynucleotide in a cell of the subject with an EON according to any one of claims 1 to 10, thereby treating the patient.

18. A method of treating HFE hemochromatosis, the method comprising administering to a patient in need thereof a therapeutically effective amount of an EON according to any one of claims 1 to 10, a vector according to claim 11, an LNP formulation according to claim 12, or a pharmaceutical composition according to claim 13.

19. A method for the deamination of a target adenosine in an HFE pre-mRNA or mRNA molecule in a cell, the method comprising the steps of:
(i) providing the cell with an EON according to any one of claims 1 to 10;
(ii) allowing uptake by the cell of the EON;
(iii) allowing annealing of the EON to the HFE pre-mRNA or mRNA molecule;
(iv) allowing an endogenous ADAR enzyme to deaminate the target adenosine in the target RNA molecule to an inosine; and optionally
(v) identifying the presence of the inosine in the target RNA molecule.

20. A method according to any one of claims 16 to 19, wherein the target adenosine is the c.845G>A mutation in the HFE pre-mRNA or mRNA molecule.

21. A method according to claim 19 or 20, wherein step (v) comprises:
a) determining the sequence of the HFE pre-mRNA or mRNA molecule;
b) assessing the presence of a wild-type HFE protein; or
c) using a functional read-out, preferably assessing a serum or plasma ferritin concentration, or a serum transferrin saturation percentage.
